(19) United States
(12) Patent Application Publication
(10) Pub. No.: US 2016/0194393 A1
(43) Pub. Date: Jul. 7, 2016

(54) METHODS AND COMPOSITIONS FOR MODULATING VOLTAGE-GATED CALCIUM CHANNEL FUNCTION

(71) Applicants: Wilfred A. Jefferies, Surrey (CA); Kyla Omilusik, Vancouver (CA); Lilian Nohara, Vancouver (CA); Kyung-Bok Choi, Vancouver (CA)

(72) Inventors: Wilfred A. Jefferies, Surrey (CA); Kyla Omilusik, Vancouver (CA); Lilian Nohara, Vancouver (CA); Kyung-Bok Choi, Vancouver (CA)

(73) Assignee: BioMmune Technologies Inc., Victoria, BC (CA)

(21) Appl. No.: 14/238,080

(22) PCT Filed: Aug. 10, 2012
(86) PCT No.: PCT/CA2012/050542

§ 371 Date: Sep. 2, 2014

Related U.S. Application Data
(60) Provisional application No. 61/522,232, filed on Aug. 10, 2011.

(51) Int. Cl. C07K 16/28; G01N 33/50

(52) U.S. Cl. CPC C07K 16/28; G01N 33/5023; C07K 2317/76

(57) ABSTRACT

Therapeutic agents targeted to voltage gated calcium channels and compositions comprising such therapeutic agents are provided, as is the use of such agents and compositions to modulate the function of haematopoietic cells expressing the voltage gated calcium channel. Also provided are methods of screening for agents that target a given voltage gated calcium channel that are suitable for use as therapeutics to modulate the activity of cells expressing the targeted voltage gated calcium channel. The agent can be, for example, an antibody, an aptamer, a peptide or a small molecule capable of binding to an ectodomain of the target voltage gated calcium channel and thus of modulating the function of the calcium channel.

### FIELD OF THE INVENTION

This invention relates to the field of therapeutics and, in particular, to therapeutics that modulate voltage-gated calcium channel (Caᵥ) function in haematopoietic cells and to methods of screening for same.

### BACKGROUND OF THE INVENTION

Calcium (Ca²⁺) ions act as universal second messengers in virtually all cell types. Voltage-gated calcium (Caᵥ) channels conduct Ca²⁺ in a variety of cell types and consist of complexes comprising the pore-forming α1 subunit, as well as at least an α2-subunit, a δ-subunit, a γ-subunit and a β-subunit. Caᵥ channels are now known to be present in many cells not traditionally considered excitable, including various haematopoietic cells.

In mammals, 10 Caᵥ family members have been grouped into 5 categories (L, P or Q, N, R, T) based on electrophysiological and pharmacological properties, each probably serving distinct cellular signaling pathways.

The expression and functions of L-type (long-lasting) Caᵥ channels in mouse and human T cells has been described (Kotturi et al., J. Biol. Chem. 278:46949-46960 (2003); Kotturi and Jefferies, Mol. Immunol. 42:1461-1474 (2005)). Four subtypes of L-type Caᵥ channels are known: Caᵥ1.1, Caᵥ1.2, Caᵥ1.3, and Caᵥ1.4. L-type Caᵥ channels have been reported in various haematopoietic cells (for review, see Suzuki, et al., Molec. Immunol. 47:640-648 (2010)).

Caᵥ1.4, an α1 Ca²⁺ channel subunit encoded by Cacnalf, has been identified as being expressed in the retina, spleen, thymus, adrenal glands, spinal cord, bone marrow, skeletal muscle and T cells of rodents and humans (Badou et al., PNAS USA 103:15529-15534 (2006); Jha et al., Nat. Immunol. 10:1275-1282 (2009); Kotturi et al., 2003, ibid; Kotturi and Jefferies, 2005, ibid; McRory et al., J. Neurosci. 24:1707-1718 (2004)).

Calcium signalling is known to play an important role in adaptive immunity. The identity and number of plasma membrane channels mediating sustained Ca²⁺ entry into T cells is unclear (Kotturi et al., Trends Pharmacol. Sci. 27:360-367 (2006)). One well-characterized mechanism of entry is through Ca²⁺ release-activated calcium (CRAC) channels (Oh-hora, Immunol. Rev. 231:210-224 (2009)). Other candidate plasma membrane Ca²⁺ channels operating in lymphocytes include the P2X receptor, transient receptor potential (TRP) cation channels, TRP vanilloid channels, TRP melastatin channels, and voltage-dependent Ca²⁺ channels (VDCC).

Two splice variants of the Caᵥ1.4 calcium channel have been identified in human T lymphocytes (Kotturi and Jefferies, 2005, ibid.). Defective survival of naïve CD8⁺ T lymphocytes in the absence of the β3 subunit of Caᵥ channels has been described and this defect was correlated with depletion of the Caᵥ1.4 subunit (Jha et al., 2009, ibid.).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide methods and compositions for modulating voltage-gated calcium channel function. In accordance with one aspect of the invention, there is provided a method for modulating the function of a cell expressing a voltage-gated calcium channel comprising contacting the cell with an agent that specifically binds to the voltage-gated calcium channel, wherein binding of the agent to the voltage-gated calcium channel modulates the activity of the channel and wherein the cell is a haematopoietic cell.

In accordance with another aspect of the invention, there is provided a method for modulating the function of a cell expressing a Caᵥ1 splice variant comprising contacting the cell with an agent that specifically binds to an ectodomain of the Caᵥ1 splice variant, wherein binding of the agent to the Caᵥ1 splice variant modulates the activity of the Caᵥ1 splice variant and wherein the cell is a haematopoietic cell.

In accordance with another aspect of the invention, there is provided a method of modulating an immune response in a subject comprising administering to the subject an effective amount of a voltage-gated calcium channel modulator, wherein the modulator binds to a voltage-gated calcium channel expressed in a haematopoietic cell.

In accordance with another aspect of the invention, there is provided a method of modulating an immune response in a subject comprising administering to the subject an effective amount of a Caᵥ1 modulator, wherein the Caᵥ1 modulator binds to an ectodomain of a Caᵥ1 splice variant expressed in a haematopoietic cell.

In accordance with another aspect of the invention, there is provided a method of screening for therapeutic agents comprising the steps of: contacting a haematopoietic cell expressing a voltage gated calcium channel with a test agent, and determining whether the test agent modulates activity of the channel, wherein a test agent that modulates activity of the channel is identified as a therapeutic agent.

In accordance with another aspect of the invention, there is provided a method of screening for therapeutic agents comprising the steps of: contacting a haematopoietic cell expressing a Caᵥ1 splice variant with a test agent, and determining whether the test agent modulates activity of the Caᵥ1 splice variant, wherein a test agent that modulates activity of the Caᵥ1 splice variant is identified as a therapeutic agent.

In accordance with another aspect of the invention, there is provided a use of an agent that specifically binds to an ectodomain of a voltage gated calcium channel expressed in haematopoietic cells, including cells of the lymphoid or myeloid lineages, to modulate cell function.

In accordance with another aspect of the invention, there is provided a use of an agent that specifically binds to an ectodomain of a Caᵥ1.4 splice variant expressed in T cells to modulate T cell function.

In accordance with another aspect of the invention, there is provided a method of suppressing an immune response in a subject comprising administering to the subject an effective amount of a Caᵥ1.4 inhibitor, wherein the Caᵥ1.4 inhibitor binds to an ectodomain of a Caᵥ1.4 splice variant expressed in T cells.

In accordance with another aspect of the invention, there is provided a use of an agent that specifically binds to an ectodomain of a Caᵥ1.4 splice variant expressed in B cells to modulate B cell function.

In accordance with another aspect of the invention, there is provided a method of suppressing an immune response in a subject comprising administering to the subject an effective amount of a Caᵥ1.4 inhibitor, wherein the Caᵥ1.4 inhibitor binds to an ectodomain of a Caᵥ1.4 splice variant expressed in B cells.

In accordance with another aspect of the invention, there is provided a method of screening for an immunosuppressant comprising the steps of: contacting T cells expressing a Caᵥ1.4 splice variant with a test agent, and determining whether the test agent modulates activity of the Caᵥ1.4 splice variant, wherein a test agent that inhibits activity of the Caᵥ1.4 splice variant is identified as an immunosuppressant.

In accordance with another aspect of the invention, there is provided a method of screening for an immunosuppressant comprising the steps of: contacting B cells expressing a Caᵥ1.4 splice variant with a test agent, and determining whether the test agent modulates activity of the Caᵥ1.4 splice variant, wherein a test agent that inhibits activity of the Caᵥ1.4 splice variant is identified as an immunosuppressant.

In accordance with another aspect of the invention, there is provided a method of modulating an immune response in a subject comprising administering to the subject an effective amount of a voltage-gated calcium channel modulator, wherein the modulator binds to a voltage-gated calcium channel expressed in a haematopoietic cell.

### BRIEF DESCRIPTION OF THE DRAWINGS

These and other features of the invention will become more apparent in the following detailed description in which reference is made to the appended drawings.

FIG. 1. Expression of Cacnalf mRNA. (A) Detection of wild type Cacnalf mRNA expression in lymphoid tissues and CD4⁺ and CD8⁺ T cells. (B) Disruption of Cacnalf gene was confirmed by RT-PCR analysis, detecting a loxP site (targeting cassette) within Cacnalf thymic transcripts of Cacnalf^{-/-} (−/−) but not wild type (+/+) mice. Detection of S15 transcripts by RT-PCR was used as a sample loading control.

FIG. 2. Caᵥ1.4 Deficiency Results in Subtle Thymic Developmental Defect, CD4⁺ and CD8⁺ T Cell Lymphopenia, and Spontaneous T Cell Immune Activation. (A) Immunoblot analysis of Caᵥ1.4 protein in whole cell extracts of WT (+/+) and Cacnalf^{-/-} (−/−) splenocytes. Weri retinoblastoma cells were used as a Caᵥ1.4-expressing positive control. GAPDH Ab staining is provided as a control for sample loading. (B) Surface proteins on WT and Cacnalf^{-/-} splenic T cells were biotinylated and immunoprecipitated with streptavidin sepharose beads. Equivalent amounts of protein were blotted with a Caᵥ1.4 Ab. A nonspecific low molecular size band on the same blot was used to confirm equal loading. (C) Cacnalf^{-/-} thymi express a reduced fraction of mature SP thymocytes, as determined by electronic gating on TCRβ^{hi} and CD24^{lo} cells (percentage is shown within rectangular gate on contour plot). (D) Caᵥ1.4 deficiency reduces the proportion of CD4 versus CD8⁺ SP thymocytes. (E) The abundance of various thymic subpopulations present in WT (n=6) and mutant (n=7) mice was determined by staining with CD4 and CD8 Abs. (F) Peripheral lymph organs including spleen, lymph nodes (LN), and blood of Cacnalf^{-/-} mice display abnormal ratios of CD4⁺ versus CD8⁺ T cells. The percentage of cells residing within each quadrant is shown within the density plot. (G) Spleens of Cacnalf^{-/-} mice exhibit greatly reduced T cell (n≧6) and B cell (n=3) numbers as compared to WT. y axis is a log scale. (H) Splenic Cacnalf^{-/-} CD4⁺ and CD8⁺ T cells express markers of acute activation and T cell memory. Error bars represent the SD. **p<0.01.

FIG. 3. Expression of markers on thymocyte populations. The amount of CD44, CD62L, TCRβ and CD69 expressed on wild type (grey shaded) and Cacnalf^{-/-} (thin-black line) DP and mature (TCRβ^{hi}) SP thymocyte subpopulations.

FIG. 4. Lymph nodes (a collection of axial, brachial, inguinal and mesenteric) of Cacnalf^{-/-} (−/−) mice exhibit greatly reduced T cell (n≧6) and B cell (n=3) cellularity as compared to wild type (+/+). Error bars represent the SD. **p<0.01; ***p<0.001.

FIG. 5. Caᵥ1.4 Is Critically Required for Both TCR- and Thapsigargin-Induced Elevations in Cytosolic-Free Ca²⁺ by Naive T Cells. WT (+/+; red line) and Cacnalf^{-/-} (−/−; blue line) splenocytes were loaded with the Ca²⁺ indicator dyes Fluo-4 and Fura Red, surface stained, and suspended in RPMI. To minimize the effects of variation in dye loading samples, intracellular Ca²⁺ amounts were plotted as a median ratio of Fluo-4/Fura Red (FL-1/FL-3) over time. (A) Electronic gating (boxed area) used to discriminate CD44^{lo} and CD44^{hi}CD4⁺ and CD8⁺ T cells is indicated within the contour plot. (B) Splenocytes were stimulated with thapsigargin (Tg) and extracellular Ca²⁺ chelated by EGTA addition at the indicated time point. (C) Splenic T cells precoated with biotinylated TCR Abs were treated with streptavidin (SA) or ionomycin (Im) at the indicated times (marked by arrows). (D) TCR stimulations were performed in the absence of free extracellular Ca²⁺. Sufficient EGTA (0.5 mM) was added to cell suspensions to chelate extracellular Ca²⁺ in RPMI (˜0.4 mM Ca²⁺), blocking cellular uptake.

FIG. 6. Caᵥ1.4 is required for TCR-induced rises in cytosolic free Ca²⁺ during Ca²⁺ limitation. (A) Wild type (+/+) and Cacnalf^{-/- (-/-) thymocytes (Total), loaded with the calcium indicator dyes Fluo-}4 and Fura Red and suspended in RPMI, were stimulated with thapsigargin (Thapsi) in the presence or absence of extracellular EGTA (0.5 mM) sufficient to chelate Ca²⁺ present in RPMI (˜0.4 mM). To minimize the effects of variation in dye loading samples, the amount of cytosolic Ca²⁺ was plotted as a ratio of FL-1/FL-3 over time. At the indicated time point, extracellular Ca²⁺ (0.5 mM) or EGTA (0.5 mM) was added midway through the stimulation. (B) Fluo-4/Fura Red-labeled thymocytes, stained with CD4 and CD8 Abs for discrimination of thymic subpopulations, were activated with TCR Abs in the presence and absence of extracellular EGTA (0.5 mM). Midway through the time course, a second stimulus, extracellular Ca²⁺ (0.5 mM) or ionomycin (1 μg/mL), was added to samples.

FIG. 7. L-Type Caᵥ1.4 Channel Mediates Ca²⁺ Entry across the Plasma Membrane of Naive T Cells. (A) Sample traces of inward barium currents recorded on WT (+/+, n=7) and Cacnalf^{-/-} (−/−; n=5) CD44^{lo}CD4⁺ and CD8⁺ T cells after TCR activation are presented. Cells were depolarized by 500 ms step pulse to +10 mV from a holding potential of −80 mV. The dotted lines indicate the baseline of current measurement (B) Current density comparison at +10 mV between WT and Cacnalf^{-/-} CD44^{lo}CD4⁺ and CD8⁺ T cells. Current values are normalized to capacitance values for each cell. (C) Current density comparison at +10 mV between untreated WT CD44^{lo} T cells (CD4⁺ T cells, n=8; CD8⁺ T cells, n=8) and those pretreated with the ectodomain-specific Caᵥ1 α1 subunit Ab (CD4⁺ T cells: n=7; CD8⁺ T cells, n=6). (D) Ectodomain-specific Caᵥ1 α1 subunit Ab immunoprecipitates Caᵥ1.4. Immunoprecipitation with an ectodomain-specific Caᵥ1 al subunit Ab was performed on WT and Cacnalf^{-/-} splenocyte extracts followed by blotting with a Caᵥ1.4-specific Ab (see Experimental Procedures). A nonspecific low molecular size band on the same blot was used to verify equivalent loading. (E and F) Sample I-V relationships for WT CD44^{lo}CD4⁺ and CD8⁺ T cells after TCR activation were obtained with a ramp pulse protocol. For display purposes, the current traces have been filtered to 1 kHz. The top inset in (E) shows the ramp pulse protocol that spans the range of −130 to 70 mV over 200 ms from a holding potential of −80 mV. The solid lines in (E) and (F) indicate the fits of whole-cell I-V relationships with the modified Boltzmann equation I=G(V−Eᵣₑᵥ)/(1+exp((Vₐ−V)/S)), where I is peak current amplitude, G is the maximum slope conductance, V is the test potential, Eᵣₑᵥ is the reversal potential, Vₐ is the half-activation potential, and S is a slope factor. The bottom insets in (E) and (F) represent averages of normalized I-V relationships obtained from WT CD44^{lo}CD4⁺ (n=5) and CD8⁺ (n=5) T cells. (G and H) Sample I-V relationships for Cacnalf^{-/-} CD44^{lo}CD4⁺ (n=6) and CD8⁺ (n=6) T cells were determined with the ramp pulse protocol as above. Error bars represent the SEM. *p<0.05.

FIG. 8. Caᵥ1.4 Function Regulates Ras-ERK Activation and NFAT Mobilization. (A) Activated Ras was measured in WT (+/+) and Cacnalf^{-/-} (−/−) thymocytes after stimulation with either TCR Ab or the DAG analog PMA with RAF-1-GST pull-down assays. Whole cell lysates (WCL) were immunoblotted for total Ras to verify equivalent protein expression. (B) Total thymocytes were stimulated with TCR Ab for the indicated period of time. Phosphorylation of ERK and JNK MAP kinases was measured by immunoblotting. Band intensities were quantified with the Odyssey software and ratios calculated for Phospho-ERK2/ERK2, Phospho-JNK1/JNK1. Unstimulated WT thymocytes were arbitrarily given a score of 1. (C) To assess ERK signaling in specific thymic subpopulations, ERK activation in WT and Cacnalf^{-/-} thymocytes after stimulation with either TCR Ab or PMA treatment for 2 min was determined via flow cytometry. Mean fluorescence intensities (MFI) for unstimulated (gray), TCR-stimulated (black), and PMA-treated (bold) cells are shown within each histogram. (D) Thymoctyes from WT and Cacnalf^{-/-} mice were incubated for 16 hr with CD3 and CD28 Abs or media alone. Immunoblotting for NFATc1 was performed on nuclear and cytoplasmic fractions and whole cell lysates (WCL). Glyceraldehyde phosphate dehydrogenase (GAPDH) or histone deacetylase-1 (HDAC1) was detected as a loading control. Band intensities were quantified and ratios calculated as above.

FIG. 9. T cell intrinsic requirement for Caᵥ1.4 function is required for normal T cell homeostasis. Irradiated recipient hosts (Thy1.2⁺Ly5.1⁺) were repopulated with Cacnalf^{-/-} (−/−; Thy1.2⁺Ly5.2⁺) and wild type (+/+; Thy1.1⁺Ly5.2⁺) bone marrow in a 1:1 ratio. (A) The origin of the Ly5.2⁺ cells in the thymus, and spleen were assessed (top panel). Cacnalf/cells (Thy1.2 gate) showed decreased survival in recipient mice as compared to wild type cells (Thy1.1 gate). Using Thy1 markers, donor lymphocytes were identified and the relative proportion of CD4⁺ and CD8⁺ T cells were determined (middle and bottom panel). The percentage of cells residing within each quadrant is shown within the density plot. (B) Percentage of donor wild type versus mutant T cells present in the thymus spleen of host mice one-month post bone marrow transfer (n=5). Error bars represent SD. ***p<0.001. (C) The relative proportion of CD44^{lo} and CD44^{hi} CD4⁺ and CD8⁺ T cells in donor lymphocyte populations are shown. The percentage of cells residing within each quadrant is shown within the density plot.

FIG. 10. Caᵥ1.4 Is an Important Regulator of Naive T Cell Homeostasis. (A) CD44 expression on splenic CD4⁺ TCRβ⁺ and CD8⁺ TCRβ⁺ T cells from WT (+/+) and Cacnalf^{-/-} (−/−) mice. (B) Cacnalf^{-/-} mice exhibit a profound reduction in CD44^{lo}CD4⁺ and CD8⁺ TCRβ⁺ T cells. (C) Cacnalf^{-/-} CD44^{lo}CD4⁺ and CD8⁺ TCRβ⁺ T cells show increased rates of spontaneous apoptosis. (D) CD62L expression on CD44^{lo}CD4⁺ and CD8⁺ TCRβ⁺ T cells. (E) Cacnalf^{-/-} CD44^{lo}CD4⁺ and CD8⁺ TCRβ⁺ T cells express reduced amounts of IL-7Rα. (F) Bcl-2 expression by CD44^{lo}CD4⁺ and CD8⁺ TCRβ⁺ T cells was measured by intracellular flow cytometry. Error bars represent the SD.

FIG. 11. (A) Cacnalf^{-/-} (−/−) CD4⁺ TCRβ^{hi} and CD8⁺ TCRβ^{hi} SP thymocytes show increased rates of spontaneous apoptosis relative to wild type (+/+). Percentage of cells present within the indicated gate is shown. (B) The amount of CD127 on wild type (grey shaded) and Cacnalf^{-/-} (thin black line) CD4⁺ TCRβ^{hi} and CD8⁺ TCRβ^{hi} SP thymocytes. Mean fluorescence intensities are shown within histograms for wild type (top) and mutant populations (bottom).

FIG. 12. Caᵥ1.4 Promotes Survival Signaling and Homeostasis-Induced T Cell Expansion. (A) WT (+/+) and Cacnalf^{-/-} (−/−) thymocytes were stimulated with the indicated concentration of IL-7 for 5 min and subsequently assessed for the capacity to phosphorylate STATS. The frequency of phospho-STATS-positive mature CD4⁺ and CD8⁺ SP thymocytes was determined by flow cytometry. (B) WT (Thy1.1⁺) and Cacnalf^{-/-} (Thy1.1⁻) naive CD4⁺ and CD8⁺ T cells, electronically gated (CD44^{lo}) as shown in FIG. 5A, were purified by cell sorting, mixed at a 1:1:1:1 ratio, and cultured with the indicated concentration of IL-7. After 24 hr incubation, cell survival was determined by staining with Annexin V conjugated to Alexa 647. (C) WT and mutant naive T cells were isolated, prepared, and cultured as in (B) except stimulated with a TCR Ab instead of IL-7. Viability was assessed after 24 hr of ex vivo culture. (D F) Naive T cells from WT (Thy1.1⁺) and Cacnalf^{-/-} (Thy1.1⁻) mice were purified, mixed at a 1:1:1:1 ratio, CFSE labeled, and coinjected into Rag1^{-/-} hosts. (D) The percentage of WT and Cacnalf^{-/-} CD4⁺ and CD8⁺ T cells is shown prior to injection. (E) CFSE dilution indicates proliferation of transferred T cells. Boxed region within dot plots indicates proliferation driven by self-MHC molecules and IL-7 (homeostatic). (F) Histograms indicating homeostatic proliferation by WT and mutant donor CD4⁺ and CD8⁺ T cells.

FIG. 13. Caᵥ1.4 Is Critically Required for Optimal Antigen-Specific CD4⁺ and CD8⁺ T Cell Immune Responses. Seven days postinfection with recombinant L. monocytogenes-OVA, WT (+/+) and Cacnalf^{-/-} (−/−) mice were sacrificed and antigen-specific T cell immune responses were assessed. (A) The percentage of CD44⁺ H-2K^{b}-OVA tetramer⁺ cells in the CD8⁺ T cell population is shown within the density plots. (B) The mean number of antigen-specific CD44⁺CD8⁺ T cells is represented (n=3). (C and D) Splenocytes from infected mice were stimulated with MHC class I (OVA₂₅₇₋₂₆₄)- and MHC class II (LLO₁₉₀₋₂₀₁)-restricted peptides and subsequently assayed for IFN-γ secretion. To determine the frequency of T cells capable of secreting IFN-γ, splenocytes were separately stimulated with TCR Ab alone. Numbers within density plots represent the percentage of IFN-γ-secreting CD4⁺ or CD8⁺ T cells. (E) Cumulative data indicating the mean numbers of antigen-specific IFN-γ-producing T cells in WT and Cacnalf^{-/-} mice (n=3). (F) CD8⁺ T cells from the spleens of infected mice were purified and incubated with ⁵¹Cr-labeled RMA-S targets that had been either untreated or pulsed with OVA₂₅₇₋₂₆₄ peptide. Error bars represent the SD. *p=0.05; ***p<0.001.

FIG. 14. Inhibition of Caᵥ1 with a blocking antibody reduces cell survival. C57Bl/6 splenocytes were incubated with (+Caᵥ1) or without (−Caᵥ1) a Caᵥ1 antibody. After 24 hours, viability was assessed by staining with Annexin V. A survival index was calculated as a ratio of the Annexin V negative cells to Annexin V positive cells. Error bars represent SD. *p<0.05; **p<0.01.

FIG. 15. Inhibition of Caᵥ1 with a blocking antibody reduces CD8⁺ and CD4⁺ T cell proliferation. C57Bl/6 splenocytes were labelled with CFSE and activated for 5 days with plate-bound CD3ε (20 μg/ml) and CD28 (5 μg/ml) antibodies with (+Caᵥ1) or without (−Caᵥ1) a Caᵥ1 antibody. Proliferation was assessed by CFSE dilution. Numbers represent the percent proliferating cells.

FIG. 16 presents the amino acid sequence of the human voltage-dependent L-type calcium channel subunit alpha-1F (Caᵥ1.4) (GenBank Accession No. NP_005174).

FIG. 17 presents the nucleotide sequence of the human voltage-dependent L-type calcium channel subunit alpha-1F splice variant (Caᵥ1.4a).

FIG. 18 presents the nucleotide sequence of the human voltage-dependent L-type calcium channel subunit alpha-1F splice variant (Caᵥ1.4b).

FIG. 19 presents a schematic representation of the predicted membrane topology for (A) the Caᵥ1.4a splice variant, and (B) the Caᵥ1.4b splice variant.

FIG. 20 presents the amino acid sequence of the human voltage-dependent L-type calcium channel subunit alpha-1F splice variant (Caᵥ1.4a).

FIG. 21 presents the amino acid sequence of the human voltage-dependent L-type calcium channel subunit alpha-1F splice variant (Caᵥ1.4b).

FIG. 22. Caᵥ1.4-deficient mice show normal B lymphocyte development in the bone marrow.

FIG. 23. Caᵥ1.4-deficient mice show altered splenic B lymphocyte maturation.

FIG. 24. Caᵥ1.4-deficiency results in altered peritoneal cavity B cell compartment.

FIG. 25. A cell-intrinsic Caᵥ1.4 function is required for normal B cell development.

FIG. 26. Caᵥ1.4-deficiency results in impaired B cell receptor- and thapsigargin-induced Ca²⁺ responses in B cells.

FIG. 27. Caᵥ1.4-deficiency results in impaired B cell receptor-induced mitochondrial Ca²⁺ responses.

FIG. 28. Caᵥ1.4-deficient B cells show defective B cell receptor-mediated activation.

FIG. 29. Caᵥ1.4-deficient B cells show reduced B cell receptor-induced proliferation.

FIG. 30. Caᵥ1.4-deficient splenic B cells show reduced expression of B cell activating factor (BAFF) receptor and lower survival rates in response to BAFF.

FIG. 31. Caᵥ1.4-deficient mice generate impaired antibody responses after immunization with TNP-Ficoll, a T cell-independent type-2 antigen.

### DETAILED DESCRIPTION OF THE INVENTION

The present invention relates to the finding, described herein, that modulation of the activity and/or expression of a voltage-gated calcium channel, such as the L-type calcium channel α1 subunits (Caᵥ1), can modify the activity of the cell expressing the channel. As different cell types express different types of voltage-gated calcium channels, agents can be designed to target the voltage-gated calcium channel expressed by a cell type of interest and can be used to specifically modulate the activity of these cells. For example, as different cell types express different subtypes and splice forms of Caᵥ1, agents can be designed to target the splice variant expressed by a cell type of interest and can be used to specifically modulate the activity of these cells.

Voltage-gated calcium channels, including but not limited to Caᵥ1 channels, may be targeted with an agent that binds to the ectodomain region of the calcium channel in order to modulate the function of the calcium channel, and thus modify the activity of the cell expressing the channel. Accordingly, in certain embodiments, the invention provides for agents targeted to an ectodomain of a voltage-gated calcium channel and the use of such agents to modulate the function of cells expressing the voltage-gated calcium channel. For example, in certain embodiments, the invention provides for agents targeted to an ectodomain of a Caᵥ1 splice variant and the use of such agents to modulate the function of cells expressing the targeted splice variant. Certain embodiments of the invention also provide for methods of screening for agents that target a given voltage-gated calcium channel that are suitable for use as therapeutics to modulate the activity of cells expressing the calcium channel. For example, in certain embodiments of the invention provide for methods of screening for agents that target a given Caᵥ1 splice variant (“Caᵥ1 modulators”) that are suitable for use as therapeutics to modulate the activity of cells expressing the targeted splice variant. The agent can be, for example, an antibody, an aptamer or a small molecule capable of binding to an ectodomain of the target voltage-gated calcium channel, including but not limited to, a Caᵥ1 splice variant and thus of modulating the function of the calcium channel. In certain embodiments, the methods, uses and compositions relate to voltage-gated calcium channels that are expressed in haematopoietic cells, such as T cells, B cells, mast cells and/or natural killer cells. In certain embodiments, the methods, uses and compositions relate to Caᵥ1 splice variants that are expressed in haematopoietic cells, such as T cells and/or B cells.

By way of example, in certain embodiments, the invention provides for an agent that targets an ectodomain of a Caᵥ1 splice variant expressed in T cells (such as Caᵥ1.4) and the use of such an agent to modulate the activity of T cells. In other embodiments, the invention provides for an agent that targets an ectodomain of a Caᵥ1 splice variant expressed in B cells (such as Caᵥ1.4) and the use of such an agent to modulate the activity of B cells.

Agents that target a voltage-gated calcium channel expressed in one or more types of haematopoietic cells, including but not limited to lymphocytes (B cells, T cells and Natural Killer cells), monocytes, macrophages and mast cells and inhibit the activity of the channel may be useful, for example, as immunosuppressants, which find application, for instance, in the treatment of autoimmune diseases, to decrease the risk of transplant rejection, and in the treatment of other disorders requiring suppression of the immune system, such as treatment of allergy. For example, agents that target an ectodomain of a Caᵥ1 splice variant expressed in T cells and/or B cells and inhibit the activity of the channel are useful, for example, as immunosuppressants, which find application, for instance, in the treatment of autoimmune diseases, to decrease the risk of transplant rejection, and in the treatment of other disorders requiring suppression of the immune system. In another example, agents that target and inhibit voltage-gated calcium channels expressed in mast cells may inhibit mast degranulation and therefore may be useful in the treatment of allergy.

In certain other embodiments, there is provided agents and methods to stimulate the activity of voltage-gated calcium channels, including but not limited to Caᵥ1 channels. Such agents and methods may be useful in the treatment of cancer and/or treatment of immune suppression.

In certain other embodiments, there is provided agents and methods which increase or decrease expression of voltage-gated channels in a cell. For example, polynucleotides which express voltage-gated channels, including but not limited to Caᵥ1 channels and vectors comprising these polynucleotides may be used to increase expression of Caᵥ1 channels.

Alternatively, polynucleotides which express antisense specific for the voltage-gated calcium channel, including but not limited to Caᵥ1 channels, may be used to decrease expression of the channels.

### DEFINITIONS

Unless defined otherwise, all technical and scientific terms used herein have the same meaning as commonly understood by one of ordinary skill in the art to which this invention belongs.

The term “antibody,” as used herein with reference to a Caᵥ1 splice variant, refers to an immunoglobulin molecule (or combinations thereof) that specifically binds to, or is immunologically reactive with, the Caᵥ1 splice variant, and includes polyclonal, monoclonal, genetically engineered and otherwise modified forms of antibodies, including but not limited to chimeric antibodies, humanized antibodies, heteroconjugate antibodies (such as bispecific antibodies, diabodies, triabodies, and tetrabodies), single chain Fv antibodies (scFv), polypeptides that contain at least a portion of an immunoglobulin that is sufficient to confer specific antigen binding to the Caᵥ1 splice variant, and antigen binding fragments of antibodies. Antibody fragments include proteolytic antibody fragments (such as F(ab′)2 fragments, Fab′ fragments, Fab′-SH fragments, Fab fragments, Fv, and rIgG), recombinant antibody fragments (such as sFv fragments, dsFv fragments, bispecific sFv fragments, bispecific dsFv fragments, diabodies, and triabodies), complementarity determining region (CDR) fragments, camelid antibodies (see, for example, U.S. Pat. Nos. 6,015,695; 6,005,079; 5,874,541; 5,840,526; 5,800,988; and 5,759,808), and antibodies produced by cartilaginous and bony fishes and isolated binding domains thereof (see, for example, International Patent Application Publication No. WO03014161).

The term “chimeric antibody,” as used herein, refers to a polypeptide comprising all or a part of the variable regions from one host species linked to at least part of the constant regions from another host species.

The term “humanized antibody,” as used herein, refers to a polypeptide comprising a modified variable region of a human antibody wherein a portion of the variable region has been substituted by the corresponding sequence from a non-human species and wherein the modified variable region is linked to at least part of the constant region of a human antibody. In one embodiment, the portion of the variable region is all or a part of the complementarity determining regions (CDRs). The term also includes hybrid antibodies produced by splicing a variable region or one or more CDRs of a non-human antibody with a heterologous protein(s), regardless of species of origin, type of protein, immunoglobulin class or subclass designation, so long as the hybrid antibodies exhibit the desired biological activity (i.e. the ability to specifically bind a Caᵥ1 protein).

The term “bispecific antibody,” as used herein, refers to an antibody that comprises a first arm having a specificity for one antigenic site and a second arm having a specificity for a different antigenic site, i.e. the bifunctional antibodies have a dual specificity.

The term “inhibit,” as used herein, means to decrease or arrest a given activity or function. In accordance with certain embodiments of the present invention, an agent is considered to inhibit an activity or function when the level of the activity or function that takes place in the presence of the agent is decreased by at least 10% when compared to the level in the absence of the agent. In some embodiments, an agent is considered to inhibit an activity or function when the level of the activity or function that takes place in the presence of the agent is decreased by at least 20%, for example, at least 25%, at least 30%, at least 40%, at least 50%, at least 60%, at least 70%, at least 75% or at least 80% when compared to the level in the absence of the agent.

The terms “therapy” and “treatment,” as used interchangeably herein, refer to an intervention performed with the intention of improving a subject's status. The improvement can be subjective or objective and is related to ameliorating the symptoms associated with, preventing the development of, or altering the pathology of a disease being treated. Thus, the terms therapy and treatment are used in the broadest sense, and include the prevention (prophylaxis), moderation, reduction, and curing of a disease at various stages. Preventing deterioration of a subject's status is also encompassed by the term. Subjects in need of therapy/treatment thus include those already having the disease as well as those prone to, or at risk of developing, the disease and those in whom the disease is to be prevented.

The term “ameliorate” includes the arrest, prevention, decrease, or improvement in one or more of the symptoms, signs, and features of the disease or disorder being treated, either temporarily or in the long-term.

The terms “subject” and “patient” as used herein refer to an animal, such as a mammal or a human, in need of treatment.

As used herein, the term “about” refers to an approximately +/−10% variation from a given value. It is to be understood that such a variation is always included in any given value provided herein, whether or not it is specifically referred to.

The use of the preposition “a” or “an” when used herein in conjunction with the term “comprising” may mean “one,” but it is also consistent with the meaning of “one or more,” “at least one” and “one or more than one.”

As used herein, the words “comprising” (and grammatical variations thereof, such as “comprise” and “comprises”), “having” (and grammatical variations thereof, such as “have” and “has”), “including” (and grammatical variations thereof, such as “includes” and “include”) or “containing” (and grammatical variations thereof, such as “contains” and “contain”) are inclusive and open-ended and do not exclude additional, unrecited elements or method steps.

It is contemplated that any embodiment discussed herein can be implemented with respect to any method, use or composition of the invention, and vice versa. Furthermore, compositions of the invention can be used to achieve methods and uses of the invention.

### Voltage-Gated Calcium Channels

In accordance with embodiments of the present invention, the target protein for the agents, uses and methods described herein is a human voltage-dependent calcium channel. In accordance with certain embodiments of the present invention, the target protein for the agents, uses and methods described herein is a human voltage-dependent calcium channel expressed in haematopoietic cells. In accordance with certain embodiments of the present invention, the target protein for the agents, uses and methods described herein is a human voltage-dependent L-type calcium channel subunit alpha-1 (Caᵥ1). Voltage-gated calcium channels are expressed in a variety of cell types. For example, Caᵥ1 is expressed in a number of different tissues including retina, spleen, thymus, adrenal gland, spinal cord, bone marrow and skeletal muscle. In accordance with one aspect of the present invention, the target protein for the agents, uses and methods described herein is a voltage-gated calcium channel, including but not limited to a Caᵥ1 splice variant (for example, a Caᵥ1.1, Caᵥ1.2, Caᵥ1.3 or Caᵥ1.4 splice variant) that is expressed in haematopoietic cells, such as cells from the myeloid lineage (including monocytes, macrophages, neutrophils, basophils, eosinophils, erythrocytes, megakaryocytes, platelets, mast cells and dendritic cells) and cells from the lymphoid lineage (including T cells, B cells and natural killer (NK) cells).

The amino acid sequences of various voltage-gated calcium channels, including but not limited to the subtypes of Caᵥ1 (Caᵥ1.1, Caᵥ1.2, Caᵥ1.3 and Caᵥ1.4) are known in the art and available from GenBank and the literature, as are the amino acid sequences of various splice forms of these proteins.

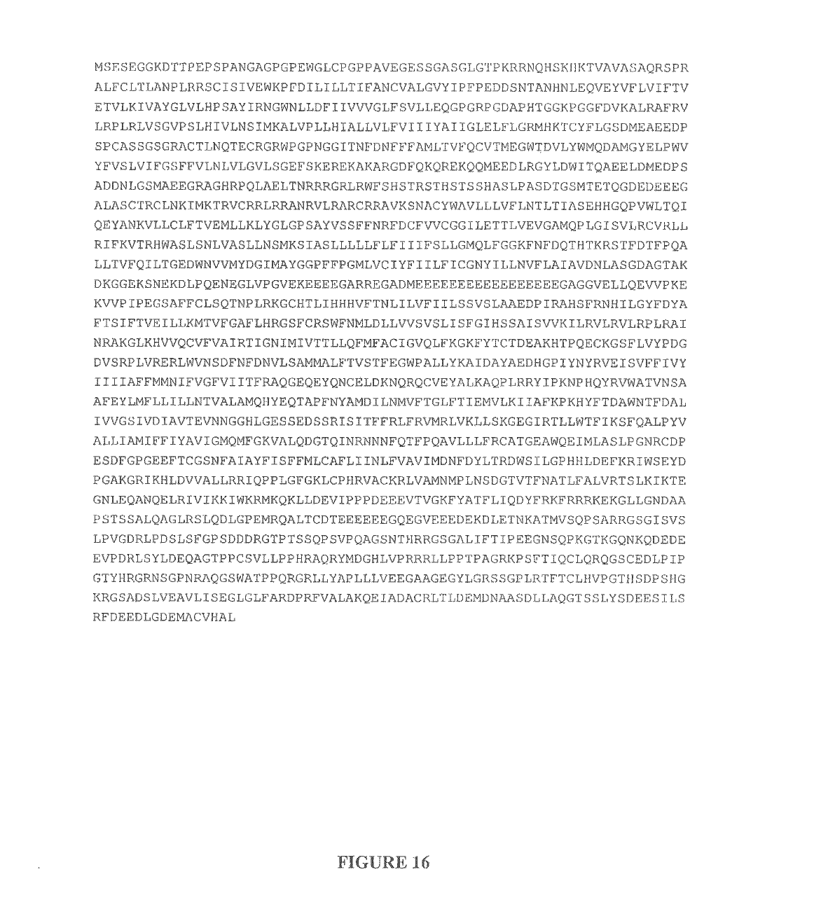

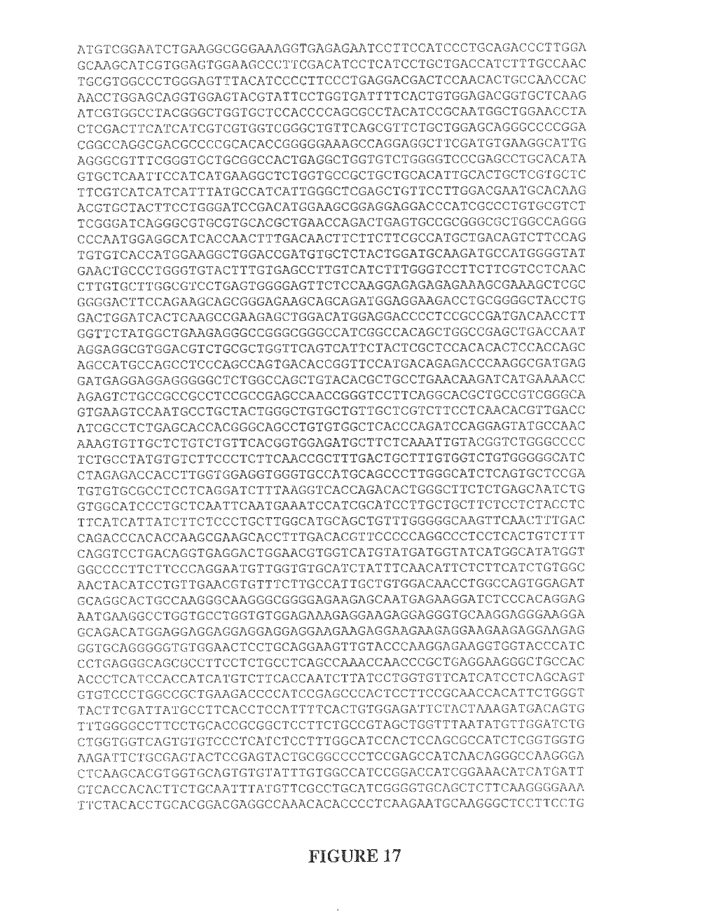

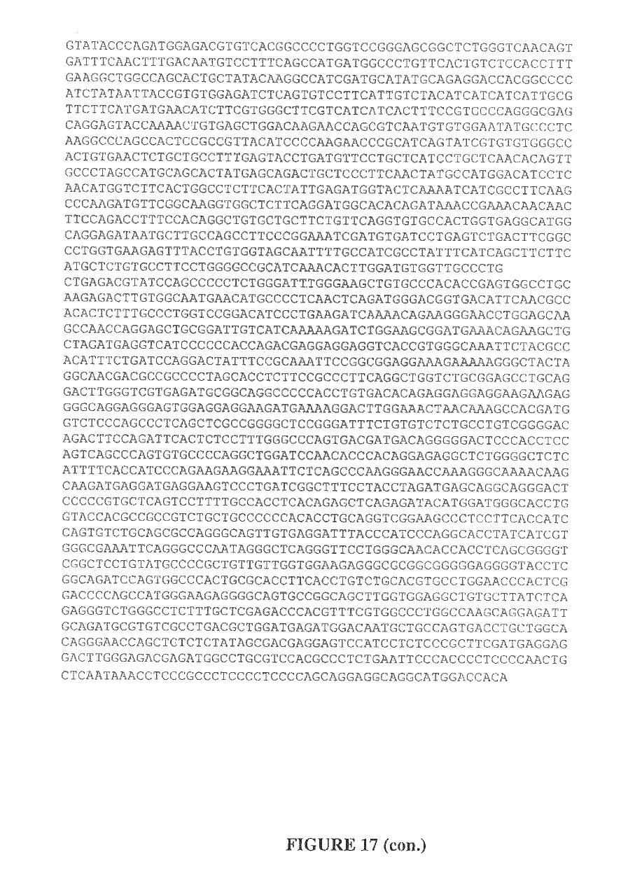

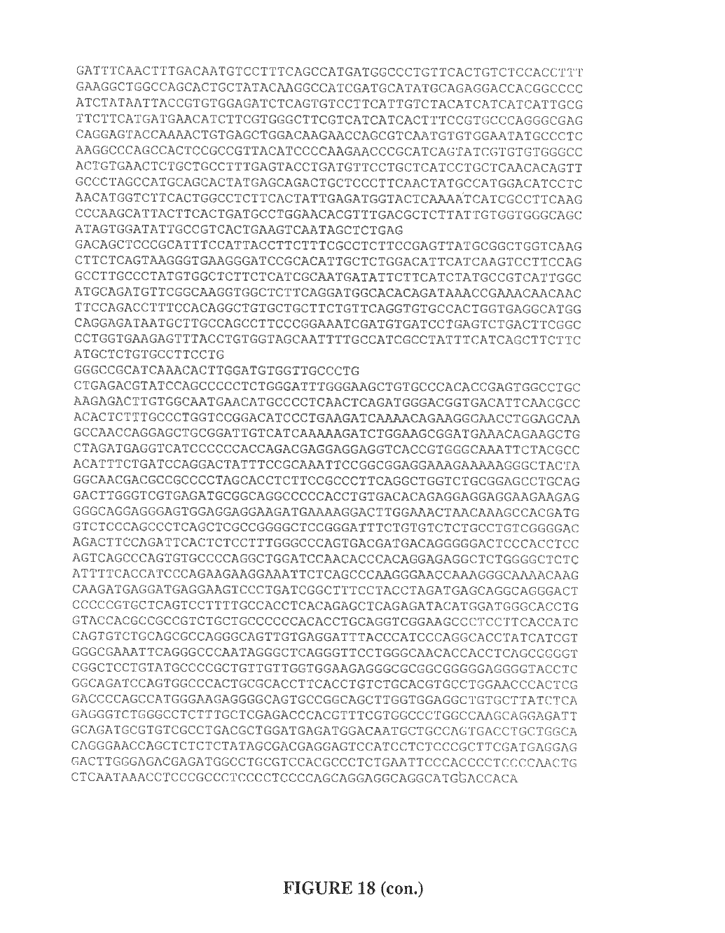

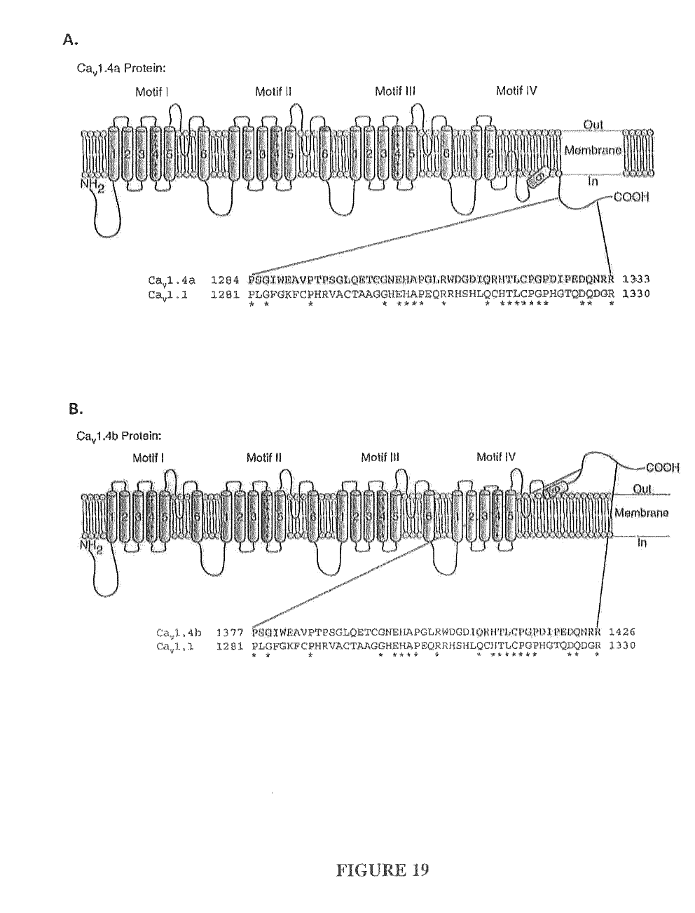

For example, the retinal form of Caᵥ1.4 is listed as the Reference Sequence in GenBank under Accession No. NP_005174 (FIG. 16). Various splice forms of this protein have been identified, including Caᵥ1.4a and Caᵥ1.4b, which are expressed in T cells (Kotturi & Jefferies, 2005, Molec. Immunol. 42:1461-1474). The sequences of Caᵥ1.4a and Caᵥ1.4b are provided herein as FIGS. 20 and 21, respectively (see also FIGS. 17 and 18, which provide the nucleotide sequences for Car 1.4a and Car 1.4b, respectively)).

If the sequence of the voltage-gated calcium channel, including but not limited to a Caᵥ1 splice variant, expressed in a cell type of interest is unknown, it can be readily determined by methods known in the art and described in various general texts (see for example, Sambrook et al., Molecular Cloning: A Laboratory Manual, 3d ed., Cold Spring Harbor Press, 2001; Ausubel et al., Current Protocols in Molecular Biology, J. Wiley & Sons, New York, N.Y., 1992 (and Supplements to 2000); Ausubel et al., Short Protocols in Molecular Biology: A Compendium of Methods from Current Protocols in Molecular Biology, 4th ed., Wiley & Sons, 1999). For example, cDNA libraries can be generated from tissue harbouring the cell type of interest using standard techniques. Alternatively, a cDNA library can be obtained from one of a variety of commercial suppliers (such as Clontech, Palo Alto, Ca.; Invitrogen, Carlsbad, Ca.). The sequence encoding the voltage-gated calcium channel, including but not limited to a Caᵥ1 subtype of interest, can be isolated by methods known in the art, for instance, by utilizing PCR amplification and sequencing techniques, such as deep sequencing that involves amplifying the transcript using common primers from the 3′ and 5′ ends using PCR or nested PCR.

In certain embodiments, the use of Illumina® DNA sequencing technology (Illumina, Inc., San Diego, Ca.) to identify the voltage-gated calcium channel, including but not limited to Caᵥ1 splice variants, expressed in a cell type of interest is contemplated. This technology provides a high-throughput, cost-effective, approach for assessing splice variation via an efficient and focused population-based strategy.

In accordance with one aspect of the present invention, therapeutic agents are targeted to an ectodomain region of the Caᵥ1 splice variant. The topology for Caᵥ1, including identification of the ectodomains, has been predicted (see, for example, Kotturi, et al., (2006), ibid., and Suzuki, et al., (2010), ibid).

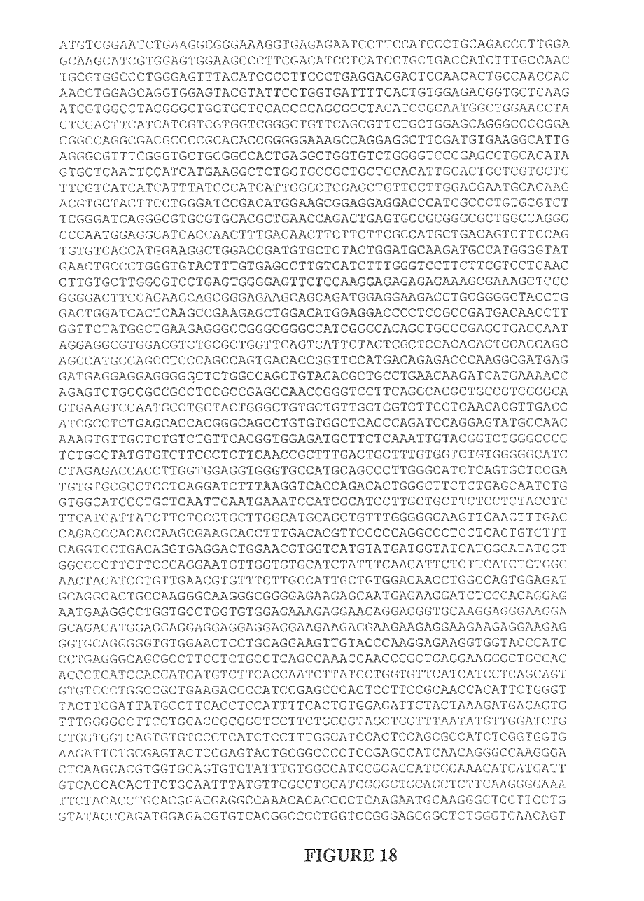

The ectodomains of certain splice variants of Caᵥ1 have been identified. For example, a channel topology for the splice variants Caᵥ1.4a and Caᵥ1.4b has been proposed (Kotturi and Jefferies (2005) ibid) and is shown in FIGS. 19A and B.

Ectodomains of a selected splice variant can be identified when necessary by standard predictive computational methods (see, for example, Coligan et al., Current Protocols in Protein Science, J. Wiley & Sons, New York, N.Y.). Alternatively, ectodomains can be identified by various surface mapping techniques, for example, by comparing antibodies capable of binding to unpermeabilized cells expressing the Caᵥ1 splice variant against a peptide library from the Caᵥ1 splice variant to determine the peptide epitopes bound by the antibody or antibodies, thus identifying sequences of the splice variant found at the surface of the cell.

In certain embodiments of the present invention, the target protein for the agents, uses and methods described herein is a Caᵥ1 splice variant that is expressed in haematopoietic cells from the lymphoid lineage (including T cells, B cells and NK cells). In some embodiments, the target protein for the agents, uses and methods described herein is a Caᵥ1.4 splice variant that is expressed in haematopoietic cells. In some embodiments, the target protein for the agents, uses and methods described herein is a Caᵥ1.4 splice variant that is expressed in haematopoietic cells from the lymphoid lineage (including T cells, B cells and NK cells).

### Therapeutic Agents

One aspect of the present invention provides for therapeutic agents that modulate the expression or activity of a voltage-gated calcium channel. In certain embodiments, therapeutic agents that modulate the expression or activity of voltage gated calcium channels expressed in haematopoietic cells are provided. In certain embodiments, therapeutic agents that modulate the expression or activity of Caᵥ1 (“Caᵥ1 modulators”) are provided. In certain embodiments, the therapeutic agents bind to and modulate the activity of Caᵥ1. In accordance with certain embodiments, the therapeutic agents target an ectodomain of the Caᵥ1 protein and thus act at the surface of the cell. Examples of suitable therapeutic agents include, but are not limited to, antibodies, aptamers, synthetic antibodies, synthetic antibody substitutes, polypeptides, peptides and small molecule therapeutics. In one embodiment, the invention provides for therapeutic agents that target and modulate the activity of Caᵥ1, that are “biologics,” for example, antibodies, aptamers, inhibitory peptides and the like. In one embodiment, polynucleotides or vectors express therapeutic agents, such as antibodies, aptamers, polypeptides and peptides.

In certain embodiments of the invention, the therapeutic agents are agents that inhibit the activity of the voltage-gated calcium channel. In certain embodiments of the invention, the therapeutic agents are agents that inhibit the activity of the Caᵥ1 (“Caᵥ1 inhibitors”). These agents may bind to and inhibit the activity of Caᵥ1. In certain embodiments of the invention, the therapeutic agents are agents that activate the activity of the voltage-gated calcium channel. In some embodiments, the therapeutic agents are agents activate the activity of Caᵥ1 (“Caᵥ1 activators”). These agents may bind to and activate the activity of Caᵥ1.

In certain embodiments of the invention, the therapeutic agent is an antibody that selectively binds the target voltage-gated calcium channel. In certain embodiments of the invention, the therapeutic agent is an antibody that selectively binds the target Caᵥ1 splice variant. The antibody may selectively bind an ectodomain of the target Caᵥ1 splice variant. As used herein, the term “selectively binds to” refers to the specific binding of one compound to another (for instance, an antibody to a Caᵥ1 protein), in which the level of binding, as measured by a standard assay (for example, an immunoassay), is statistically significantly higher than the background control for the assay. For example, when performing an immunoassay, a control could include a reaction well/tube that contains antibody alone (for example, in the absence of target protein), wherein an amount of reactivity (such as, non-specific binding to the well/tube) by the antibody in the absence of the target protein is considered to be background.

Binding can be measured using a variety of methods standard in the art, including, but not limited to: Western blot, immunoblot, enzyme-linked immunosorbant assay (ELISA), radioimmunoassay (MA), immunoprecipitation, surface plasmon resonance, chemiluminescence, fluorescent polarization, phosphorescence, immunohistochemical analysis, matrix-assisted laser desorption/ionization time-of-flight mass spectrometry, microcytometry, microarray, microscopy, fluorescence activated cell sorting (FACS), and flow cytometry.

Antibodies that specifically bind to a voltage-gated calcium channel, such as a Caᵥ1 splice variant, may be generated by various standard methods known in the art. Polyclonal antibodies, for example, can be produced by administering the Caᵥ1 splice variant or a fragment thereof to a suitable host animal such as a rabbit, mouse, rat, or the like, in order to induce the production of sera containing polyclonal antibodies specific for the administered protein. Various adjuvants known in the art may be used if desired to increase the immunological response, depending on the host species, and include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, BCG (bacille Calmette-Guerin) and corynebacterium parvum.

Monoclonal antibodies can be prepared, for example, through the use of hybridoma, recombinant, or phage display technologies, or a combination thereof. For instance, monoclonal antibodies can be produced using hybridoma techniques such as those taught in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N. Y., 1981).

By way of example, mice can be immunized with the Caᵥ1 splice variant or a fragment thereof or a cell expressing the Caᵥ1 splice variant or fragment. Once an immune response is detected, for example by detecting antibodies specific for the Caᵥ1 splice variant or fragment in the mouse serum, the mouse spleen is harvested and splenocytes isolated. The splenocytes are then fused by well-known techniques to suitable myeloma cells. Hybridomas are selected and cloned by limited dilution. The hybridoma clones are then assayed by methods known in the art for cells that secrete antibodies capable of binding the Caᵥ1 splice variant or fragment. Ascites fluid, which generally contains high levels of antibodies, can be generated by immunizing mice with positive hybridoma clones.

Antibody fragments which recognize specific epitopes of a voltage-gated calcium channel, such as a Caᵥ1 splice variant, can be generated by known techniques. For example, Fab and F(ab′)2 fragments can be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

Antibodies can also be generated, for example, using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (for example, human or murine). Phage expressing an antigen-binding domain that binds the Caᵥ1 splice variant can be selected or identified with the Caᵥ1 splice variant or a fragment thereof, for example, using a labeled protein or fragment, or the protein or fragment bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used include, for example, those described in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); International Patent Application No. PCT/GB91/01134; International Patent Application Publication Nos. WO 90/02809; WO 91/10737, WO 92/01047, WO 92/18619, WO 93/11236, WO 95/15982 and WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

After phage selection, the antibody coding regions from the phage can be isolated and used to generate whole antibodies, including human antibodies, or a desired antigen binding fragment, and expressed in an appropriate host cell, including mammalian cells, insect cells, plant cells, yeast, and bacteria. For example, techniques to recombinantly produce Fab, Fab′ and F(ab′)2 fragments are described in International Patent Application Publication No. WO 92/22324; Mullinax et al., BioTechniques 12(6):864-869 (1992); Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

Methods for producing chimeric antibodies are known in the art. For example, see Morrison, Science 229:1202 (1985); Oi et al., BioTechniques 4:214 (1986); Gillies et al., J. Immunol. Methods 125:191-202 (1989), and U.S. Pat. Nos. 5,807,715, 4,816,567 and 4,816,397.

Humanized antibodies are antibody molecules from non-human species antibody that binds the desired antigen having one or more complementarity determining regions (CDRs) from the non-human species and a framework region from a human immunoglobulin molecule. Often, framework residues in the human framework regions will be substituted with the corresponding residue from the CDR donor antibody to alter, preferably improve, binding to the target protein or protein fragment. These framework substitutions are identified by methods well known in the art, for example, by modeling of the interactions of the CDR and framework residues to identify framework residues important for binding and sequence comparison to identify unusual framework residues at particular positions (see, for example, U.S. Pat. No. 5,585,089, and Riechmann et al., Nature 332:323 (1988)). Antibodies can be humanized using a variety of techniques known in the art including, for example, CDR-grafting (International Patent Application Publication No. WO 91/09967, and U.S. Pat. Nos. 5,225,539, 5,530,101 and 5,585,089), veneering or resurfacing (Padlan, Molecular Immunology 28(4/5):489-498 (1991); Studnicka et al., Protein Engineering 7(6):805-814 (1994), and Roguska et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111, and International Patent Application Publication Nos. WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with the Caᵥ1 splice variant or a fragment thereof. Monoclonal antibodies directed against the Caᵥ1 splice variant or fragment can be obtained from the immunized, transgenic mice using conventional hybridoma technology. The human immunoglobulin transgenes harbored by the transgenic mice rearrange during B cell differentiation and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, for example, International Patent Application Publication Nos. WO 98/24893, WO 92/01047, WO 96/34096 and WO 96/33735; European Patent No. 0 598 877; U.S. Pat. Nos. 5,413,923, 5,625,126, 5,633,425, 5,569,825, 5,661,016, 5,545,806, 5,814,318, 5,885,793, 5,916,771 and 5,939,598. In addition, companies such as Abgenix, Inc. (Freemont, Ca.) and Genpharm (San Jose, Ca.) can be engaged to provide human antibodies directed against a selected protein using technology similar to that described above.

Completely human antibodies which recognize a selected epitope can also be generated using a technique referred to as “guided selection.” In this approach a selected non-human monoclonal antibody, such as a mouse antibody, is used to guide the selection of a completely human antibody recognizing the same epitope (see Jespers et al., Bio/technology 12:899-903 (1988)).

Antibodies contemplated by the invention include, in some embodiments, derivatives that are modified by the covalent attachment of an additional molecule to the antibody in such a way that additional molecule does not prevent the antibody from binding to its target protein. By way of example, the antibody derivatives may include antibodies that have been modified by glycosylation, acetylation, pegylation, phosphorylation, amidation, derivatization by known protecting/blocking groups, proteolytic cleavage, or linkage to a cellular ligand or other protein, for example. Derivatives that comprise antibodies including one or more non-classical amino acids are also contemplated in some embodiments.

In certain embodiments of the invention, the therapeutic agent is an aptamer that selectively binds an ectodomain of the Caᵥ1 splice variant. The aptamer may selectively binds an ectodomain of the Caᵥ1 splice variant. Aptamers include single-stranded nucleic acid molecules (such as DNA or RNA) that assume a specific, sequence-dependent shape and bind to the target protein with high affinity and specificity. Aptamers are generally 100 nucleotides or less in length, for example, 75 nucleotides or less, or 50 nucleotides or less in length (such as between about 10 and about 100 nucleotides, or between about 10 and about 50 nucleotides). In some embodiments, the aptamer may be a mirror-image aptamer (also called a SPIEGELMER™). Mirror-image aptamers are high-affinity L-enantiomeric nucleic acids (for example, L-ribose or L-2′-deoxyribose units) that display high resistance to enzymatic degradation compared with D-oligonucleotides (such as, aptamers). The target binding properties of aptamers and mirror-image aptamers are designed by an in vitro-selection process starting from a random pool of oligonucleotides, as described for example, in Wlotzka et al., PNAS 99(13):8898-90 (2002).

In some embodiments, the aptamer may be a peptide aptamer. Peptide aptamers include a peptide loop (for example, which is specific for the Caᵥ1 splice variant) attached at both ends to a protein scaffold. This double structural constraint greatly increases the binding affinity of the peptide aptamer to levels comparable to those of antibody binding. The variable loop length is typically between about 8 and about 20 amino acids (for example, between about 8 and about 15, or about 8 and about 12 amino acids), and the scaffold is a protein which is suitably stable, soluble, small, and non-toxic. Examples of suitable proteins include, but are not limited to, thioredoxin-A, stefin A triple mutant, green fluorescent protein, eglin C, or cellular transcription factor Sp1. Peptide aptamer selection can be made using different systems, such as the yeast two-hybrid system (for example, Gal4 yeast-two-hybrid system) or the LexA interaction trap system.

In some embodiments the therapeutic agent is a synthetic antibody or synthetic antibody substitute, both of which can be prepared by methods known in the art (see, for example, Sidhu and Fellouse, Nature Chemical Biology 2:682-688 (2006)). Synthetic antibody substitutes are generally peptide-based.

In certain embodiments, the therapeutic agents are binding peptides, which can be identified, for example, by phage display or yeast two-hybrid techniques as is known in the art.

Some embodiments of the invention provide for therapeutic agents that are small molecules, which can be obtained by screening commercially available combinatorial libraries or natural product libraries, for example.

The therapeutic agents can be tested for their ability to target and modulate the activity of Caᵥ1 using standard techniques, such as those described below in the section entitled “Methods of Screening for Therapeutic Agents.”

Certain embodiments of the present invention provides for voltage-gated calcium channel modulators that target a voltage-gated calcium channel expressed in a haematopoietic cell of lymphoid lineage (for example, a B cell, T cell or NK cell) or myeloid lineage. One embodiment of the present invention provides for Caᵥ1 modulators that target a Caᵥ1 splice variant expressed in a haematopoietic cell of lymphoid lineage (for example, a B cell, T cell or NK cell). These modulators may target the ectodomain of the Caᵥ1 splice variant. In certain embodiments, these therapeutic agents are Caᵥ1 inhibitors and find use as immunosuppressants. In certain other embodiments, these therapeutics are inhibitors of voltage-gated calcium channels expressed on mast cells and may find use in the treatment of allergy.

In some embodiments, the present invention provides for Caᵥ1 modulators that target a Caᵥ1.4 splice variant expressed in a haematopoietic cell of lymphoid lineage (for example, a B cell, T cell or NK cell). These modulators may target the ectodomain of the Caᵥ1.4 splice variant. In certain embodiments, these therapeutic agents are Caᵥ1.4 inhibitors and find use as immunosuppressants.

Also provided are pharmaceutical compositions comprising a therapeutic agent that binds to and modulates the activity of Caᵥ1 and one or more pharmaceutically acceptable carriers, diluents, excipients and/or adjuvants. If desired, other active ingredients may be included in the compositions. These other active ingredients may include for example other known immune modulatory compounds. Such compositions are formulated for administration to an animal, including humans. The pharmaceutical compositions can be formulated for administration by a variety of routes. For example, the compositions can be formulated for oral, topical, rectal or parenteral administration or for administration by inhalation or spray.

The term parenteral as used herein includes subcutaneous injections, intravenous, intramuscular, intrathecal, intrasternal injection or infusion techniques.

Various pharmaceutical compositions for administration by a variety of routes and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philidelphia, Pa. (2000).

### Methods of Screening for Therapeutic Agents

One aspect of the present invention provides for methods of screening for agents that target a given voltage-gated calcium channel that are suitable for use as therapeutics to modulate the activity of cells expressing the splice variant. In certain embodiments of the present invention provides for methods of screening for agents that target a given Caᵥ1 splice variant that are suitable for use as therapeutics to modulate the activity of cells expressing the splice variant.

In general, the methods of screening comprise contacting a haematopoietic cell expressing a voltage-gated calcium channel of interest, such as a Caᵥ1 splice form of interest, with a candidate therapeutic agent, and determining whether the candidate therapeutic agent modulates activity of the calium channel. Appropriate cells include, for example, mast cells, monocytes, macrophages, neutrophils, basophils, eosinophils, erythrocytes, megakaryocytes, platelets, dendritic cells, T cells, B cells and NK cells.

In certain embodiments, the methods further comprise an initial step or steps of identifying the Caᵥ1 splice variant expressed in the target cell or tissue of interest. This may be achieved, for example, as described in the section “Caᵥ1 Splice Variants” above. In some embodiments, the methods also comprise the step of identifying the ectodomains of a selected splice variant that can be targeted by the candidate therapeutic agent, as also described in the section “Caᵥ1 Splice Variants.”

Modulation of the activity of the Caᵥ1 splice variant can be assessed for example at the level of calcium channel activity or at the level of cell function.

In certain embodiments, the methods of screening comprise assessing the ability of the candidate compound to modulate calcium channel activity. In some embodiments, the methods comprise assessing the ability of the candidate compound to inhibit calcium channel activity.

Calcium channel activity can be determined using various methods known in the art for assessing calcium flux into a cell or across a membrane, for example, by voltage clamp electrophysiology methods (in particular, whole cell “patch clamp” assays) and fluorescence-based assays.

For voltage clamp electrophysiology recording, a glass micropipette breaks the cell membrane to connect the pipette lumen with the cytoplasm. This way the membrane potential across the plasma membrane can be measured. When the calcium channel is activated and calcium enters the cell across the membrane, the membrane potential is altered and this is measured through this method. “Patch-clamp” assays are described, for example, in Molnár and Hickman, Patch-clamp methods and protocols, Humana Press (2007).

Fluorescence-based assays can be used to measure increases in calcium concentrations in the cell. Briefly, cells are incubated with a calcium sensitive dye (for example, Fluro-4 or Fura-red, commercially available from Invitrogen Life Technologies) that can cross the plasma membrane and reside in the cytoplasm of the cell. Upon activation of calcium channels that allows calcium to enter the cell across the membrane, the calcium will bind the dye and alter its fluorescence properties. For example Fluro-4 dye will increase in fluorescence while Fura-red dye will decrease in fluorescence. The change in dye fluorescence properties can be measured and correlated to the increase in cytoplasmic calcium concentration or calcium flux. Fluroescence-based assay methods are described, for example, in June and Moore, Measurement of Intracellular Ions by Flow Cytometry. Current Protocols in Immunology. 5.5.1-5.5.20 (2004)).

In addition, various commercial kits are available for measurement of calcium flux and can be employed in the present methods, for example, the Fluo-4 Direct™ Calcium Assay Kit (Invitrogen, Carlsbad, Ca.) and BD™ Calcium Assay Kit (BD BioSciences).

A substantial change in calcium flux relative to control indicates that the candidate agent modulates calcium channel activity of the Caᵥ1 splice variant A control can be a known value indicative of calcium flux in a sample, such as a cell, not treated with the candidate agent. For example, a decrease in calcium channel activity by at least about 20%, at least about 30%, at least about 40%, at least about 50%, at least about 60%, at least about 70%, at least about 80%, or at least about 90% as compared to a control indicates that the candidate agent inhibits calcium channel activity, and thus the candidate agent is an inhibitor of Caᵥ1 activity. In contrast, an increase in calcium channel activity, for example, by at least about 20%, at least about 30%, at least about 40%, at least about 50%, at least about 60%, at least about 70%, at least about 80%, or at least about 90%, as compared to a control indicates that the candidate agent activates calcium channel activity, and thus the candidate agent is an activator of Caᵥ1 activity.

Appropriate functional assays can be readily determined by one skilled in the art taking into consideration the cell type involved. For example, cell survival, cell proliferation, cell differentiation and/or cell activation could be assessed by standard techniques. For example, induction of transcription factors (such as NFkB or NFAT), cytokine secretion or cytolytic ability could be assessed using techniques known in the art. Suitable assays to assess immune function of various haematopoietic cells are know in the art.

Methods of conducting such assays are well known in the art (see, for example, Short Current Protocols in Immunology: A Compendium of Methods from Current Protocols in Immunology, 2005, John Wiley & Sons Inc. New Jersey; Mast Cells: Methods and Protocols, by Krishnaswamy and Chi, 2005, Humana Press; and Neutrophil Methods and Protocols Series: Methods in Molecular Biology, Vol. 412, Quinn, et al. (Eds.) 2007, Humana Press).

In certain embodiments of the invention, the methods comprise identifying candidate compounds that are inhibitors of voltage-gated calcium channel activity. In certain embodiments of the invention, the methods comprise identifying candidate compounds that are inhibitors of Caᵥ1 activity. In certain embodiments of the invention, the methods comprise identifying candidate compounds that are modulators of Caᵥ1.4 activity. In certain embodiments of the invention, the methods comprise identifying candidate compounds that are inhibitors of Caᵥ1.4 activity.

In some embodiments of the invention, the methods of screening comprise contacting a B cell, T cell, thymocyte or splenocyte expressing the Caᵥ1 splice variant of interest with a candidate therapeutic agent and assessing the ability of the candidate agent to inhibit calcium channel activity. In accordance with this embodiment, a candidate agent that inhibits the calcium channel activity can be selected as a therapeutic agent suitable for use as an immunosuppressant.

In certain embodiments of the invention, the methods comprise identifying candidate compounds that are stimulators of Caᵥ1 activity. In certain embodiments of the invention, the methods comprise identifying candidate compounds that are stimulators of Caᵥ1.4 activity.

In some embodiments of the invention, the methods of screening comprise contacting a B cell, T cell, thymocyte or splenocyte expressing the Caᵥ1 splice variant of interest with a candidate therapeutic agent and assessing the ability of the candidate agent to stimulate calcium channel activity.

### Uses of the Therapeutic Agents

One aspect of the invention provides for use of the therapeutic agents to modulate the activity of haematopoietic cells expressing the targeted voltage-gated calcium channel, including but not limited to a Caᵥ1 splice variant.

In certain embodiments, the therapeutic agents are targeted to a voltage-gated calcium channel that is expressed in haematopoietic cells of the lymphoid lineage and can be used to modulate immune function. In certain embodiments, the therapeutic agents are targeted to a Caᵥ1 splice variant that is expressed in haematopoietic cells of the lymphoid lineage and can be used to modulate immune function. In some embodiments, the therapeutic agents inhibit the activity of a voltage-gated calcium channel that is expressed in haematopoietic cells of the lymphoid lineage and can be used to suppress an immune response (for example in order to treat autoimmune diseases, to decrease the risk of transplant rejection). In some embodiments, the therapeutic agents inhibit the activity of the Caᵥ1 splice variant and can be used to suppress an immune response (for example in order to treat autoimmune diseases, to decrease the risk of transplant rejection). In some embodiments, the therapeutic agents inhibit the activity of the Caᵥ1.4 splice variant and can be used to suppress an immune response (for example in order to treat autoimmune diseases, to decrease the risk of transplant rejection).

In certain embodiments, the therapeutic agents are targeted to a voltage-gated calcium channel that is expressed in haematopoietic cells of the myeloid lineage and can be used to modulate immune function.

In some embodiments, the therapeutic agents increase the activity of a voltage-gated calcium channel expressed in haematopoietic cells and can be used to increase an immune response (for example, in an immunocompromised subject). In some embodiments, the therapeutic agents increase the activity of the Caᵥ1 splice variant and can be used to increase an immune response (for example, in an immunocompromised subject). In some embodiments, the therapeutic agents increase the activity of the Caᵥ1.4 splice variant and can be used to increase an immune response.

In some embodiments, the therapeutic agents are targeted to a voltage-gated calcium channel that is expressed in T cells and can, therefore, be used to modulate T cell activity. In some embodiments, the therapeutic agents are targeted to a Caᵥ1.4 splice variant that is expressed in T cells and can, therefore, be used to modulate T cell activity. Certain embodiments provide for the use of therapeutic agents targeted to a Caᵥ1.4 splice variant that is expressed in T cells to inhibit binding of the T cell to antigen. Some embodiments provide for the use of therapeutic agents targeted to a Caᵥ1.4 splice variant that is expressed in T cells to inhibit T cell maturation. Such therapeutic agents have application, for example, as immunosuppressants, which can be used to treat autoimmune diseases, to decrease the risk of transplant rejection, and to treat other disorders requiring suppression of the immune system.

In some embodiments, the therapeutic agents are targeted to a voltage-gated calcium channel that is expressed in B cells and can, therefore, be used to modulate B cell activity. In some embodiments, the therapeutic agents are targeted to a Caᵥ1.4 splice variant that is expressed in B cells and can, therefore, be used to modulate B cell activity. Certain embodiments provide for the use of therapeutic agents targeted to a Caᵥ1.4 splice variant that is expressed in B cells to inhibit BCR-mediated activation and/or BCR-induced proliferation. Some embodiments provide for the use of therapeutic agents targeted to a Caᵥ1.4 splice variant that is expressed in B cells to inhibit B cell maturation. Such therapeutic agents have application, for example, as immunosuppressants, which can be used to treat autoimmune diseases or impair generation of an antibody response, and to treat other disorders requiring suppression of the immune system.

Examples of autoimmune diseases that may be treated in accordance with certain embodiments of the invention include, but are not limited to, inflammatory (rheumatoid) arthritis, Hashimoto's thyroiditis, pernicious anemia, inflammatory bowel disease (Crohn's disease and ulcerative colitis), psoriasis, renal fibroses, pulmonary fibroses, hepatic fibroses, Addison's disease, Type I diabetes, systemic lupus erythematosus (SLE), dermatomyositis, Sjogren's syndrome, multiple sclerosis, myasthenia gravis, Reiter's syndrome, and Grave's disease. Clinical measures of response can be measured for each of these diseases. For example, a reduction in pain, reduction in inflammation of tissues (for example, joints), improved tissue (for example, kidney) function, or improved ability to digest food can serve as indicators of successful immunosuppression.

Certain embodiments contemplate the administration of a therapeutic agent targeted to a voltage-gated calcium channel expressed in haematopoietic cells in conjunction with a known anti-inflammatory agent or immunosuppressive agent. Certain embodiments contemplate the administration of a therapeutic agent targeted to a T cell Caᵥ1.4 splice variant in conjunction with a known anti-inflammatory agent or immunosuppressive agent. Certain embodiments contemplate the administration of a therapeutic agent targeted to a B cell Caᵥ1.4 splice variant in conjunction with a known anti-inflammatory agent or immunosuppressive agent. Examples of immunosuppressive agents include non-steroidal anti-inflammatory agents (such as diclofenac, diflunisal, etodolac, flurbiprofen, ibuprofen, indomethacin, ketoprofen, ketorolac, nabumetone, naproxen, oxaprozin, piroxicam, sulindac, tolmetin, celecoxib, or rofecoxib), steroids (such as cortisone, dexamethasone, hydrocortisone, methylprednisolone, prednisolone, prednisone, or triamcinolone) and immunosuppressive agents (such as cyclosporin, tacrolimus, mycophenolic acid, or sirolimus). Other examples include biological response modifiers (such as Kineret® (anakinra), Enbrel® (etanercept), or Remicade® (infliximab)), disease-modifying antirheumatic drugs (DMARD) (such as Arava® (leflunomide)), Hyalgan® (hyaluronan) and Synvisc® (hylan G-F20).

Certain embodiments of the invention provide for the use of therapeutic agents that increase the activity of the a voltage-gated calcium channel expressed in haematopoeitic cells, such as a Caᵥ1 splice variant, to increase an immune response in an immunocompromised subject, for example to treat or prevent an opportunistic infection in an immunocompromised subject. Immunocompromised subjects are more susceptible to opportunistic infections, for example viral, fungal, protozoan, or bacterial infections, prion diseases, and certain neoplasms. Those who can be considered to be immunocompromised include, but are not limited to, subjects with AIDS (or HIV positive), subjects with severe combined immune deficiency (SCID), diabetics, subjects who have had transplants and who are taking immunosuppressives, and those who are receiving chemotherapy for cancer. Immunocompromised individuals also include subjects with most forms of cancer (other than skin cancer), sickle cell anemia, cystic fibrosis, those who do not have a spleen, subjects with end stage kidney disease (dialysis), and those who have been taking corticosteroids on a frequent basis by pill or injection within the last year. Subjects with severe liver, lung, or heart disease also can be immunocompromised.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Caᵥ1.4 Calcium Channel Regulates T Cell Receptor Signaling and Naïve T Cell Homeostasis

The following experiments were carried out to determine the physiological functions of Caᵥ1.4 in T cell biology.

### Experimental Methods:

Total RNA Extraction and RT-PCR.

Total RNA was extracted from the various samples using the Trizol® reagent (Invitrogen) as directed by the manufacturer. Isolated RNA was treated with DNase I to remove contaminating DNA. One microgram of total RNA was used to synthesize first strand cDNAs with random primers and superscript II (Invitrogen). To detect Cav1.4 in tissues an initial PCR was performed with sense primer (5′-CAT ACT GGA GGA AAG CCA GGA-3′) and anti-sense primer (5′-TGG AGT GTG TGG AGC GAG TAG A-3′). A subsequent nested PCR amplification was done with sense primer (5′-GAC GAA TGC ACA AGA CAT GC-3′) and anti-sense primer (5′-CAA GCA CAA GGT TGA GGA CA-3′). To detect the Cav1.4 mutated mRNA the first round PCR was performed with sense primer (5′-CATACTGGADGGAAAGCCAGGA-3′) and anti-sense primer (5′CGTC CCTCTTCAGCAAGAGAA-3′). A second nested PCR was performed with sense primer (5′-G CCCATAACTICGTATAATGTATGC-3′) and anti-sense primer (5′-CAAGCACAAGGTTGA GGACA-3′).

Antibodies (Abs).

Monoclonal antibodies used for flow cytometry against CD3ε (2C11), CD4 (GK1.5), CD8a (53-6.7), CD8b (53.58), TCRβ (H57-597), CD19 (ebio1D3), CD24(M1/69), CD25 (PC61.5), CD44 (IM7), CD62L (MEL-14), CD69 (H1.2F3), CD127 (A7R34), Thy1.1 (HIS51), Thy1.2 (53-2.1), CD45.2 (104), PD-1 (J43), PD-L (MIH5) and CCR7 (EBI-1) were purchased from eBioscience. The following antibodies were used for immunoblotting: rabbit polyclonal anti Caᵥ1.4 (McRory et al., 2004), anti Phospho-p44 and p42 MAPK (9101, Cell Signaling), anti ERK2 (sc-154, Santa Cruz), anti Phospho-JNK (9251, Cell Signaling), anti JNK (9252, Cell Signaling), anti-NFATc1 (7A6, Thermo Scientific), anti-GAPDH (MAB374, Chemicon) and anti-HDAC1 (10E2, Santa Cruz).

Bone Marrow Transfer Experiments.

Bone marrow (BM) cells were prepared from thigh bone extracts of Thy1.1 wild type (Thy1.1⁺CD45.2⁺) or Cacnalf (Thy1.2⁺CD45.2⁺) mice. Mature T cells were stained with biotinylated Thy1.1 or anti-Thy1.2 Abs and subsequently depleted with streptavidin-linked Dynabeads (Invitrogen). Wild type and mutant BM cells were then mixed 50:50 before being transferred intravenously into sub-lethally irradiated (1000 rads) CD45.1⁺ hosts (Thy1.2⁺CD45.1⁺). Cells from spleen and thymus were recovered 30 days after adoptive transfer; Thy1.1, Thy1.2 and CD45.2 were the basis for discriminating wild type and mutant donor cells.

Mice.

Cacnalf^{-/-} mice that have been previously described (Mansergh et al., 2005) were bred onto C57BL/6J (B6) background for at least 13 generations. B6, B6.PL-Thy1^{a}/5Cy (Thy1.1⁺), B6.SJL-Ptprca Pep3b/BoyJ (Ly5.1⁺), and B6.Rag1^{-/-} were acquired from the Jackson Laboratory (Bar Harbor, Me.). All studies followed guidelines set by both the University of British Columbia's Animal Care Committee and the Canadian Council on Animal Care.

Flow Cytometry.

All Ab incubations were done on ice. Annexin V-PE (BD Biosciences), anti-Bcl-2 (3F11; BD Biosciences), and isotype control Ab staining was conducted as previously described (Priatel et al., 2000, 2006). Data were acquired with either FACScan or FACSCalibur and CellQuest software (BD Biosciences) or LSRII and FACSDiVa software (BD Biosciences). Data were analyzed with Flowjo software (Treestar, Inc).

Ca²⁺ Flux Assay.

Splenocytes or thymocytes (10⁷ cells/mL) in HBSS (Hank's balanced salt solution) with 2% FCS were labeled with 1 μM Fluo-4, 2 μM Fura Red, and 0.02% pluronic (all from Invitrogen) for 45 min at room temperature. After washing, cells were stained with CD44-APC, CD8-APC-eFluor 780 (eBioscience), and CD4-PE Abs for 20 min on ice. Samples were suspended in RPMI (contains ˜0.4 mM Ca²⁺) and prewarmed for 15 min at 37° C. Thapsigargin (1 μM) and ionomycin (1 μg/mL) stimulations and the adding back of free extracellular Ca²⁺ (0.5 mM) were performed as described previously (Liu et al., 1998). Chelation of extracellular Ca²⁺ was carried out by supplementation of RPMI media with 0.5 mM EGTA. For TCR stimulations, splenocytes precoated with 5 μg/mL of biotinylated CD3ε Ab (clone 145-2C11; eBioscience) were activated by the addition of 20 μg/mL streptavidin. Ca²⁺ flux data was acquired on a BD LSR II flow cytometer with FACSDiva software or BD FACSCalibur with CellQuest software and analyzed with Flowjo (Treestar, Inc), electronically gating on the indicated T cell subsets and plotting Fluo-4/Fura Red ratios versus time.

Electrophysiological Assays.

Single-cell suspensions generated from lymph nodes and spleens of WT and Cacnalf^{-/-} mice were stained with CD44 (IM7), CD4 (GK1.5), and CD8a (53-6.7) Abs and subsequently, CD44^{lo}CD4⁺ and CD8⁺ T cells were isolated with a BD FACSAria. The vast majority (>99%) of sorted CD44^{lo} T cells were considered naive because they were CD62L^{hi}. TCR stimulations were performed as described for Ca²⁺ flux assays. For Ca²⁺ channel blocking experiments, cells were preincubated with an Ab specific to the ectodomains of Caᵥ1.3 and Caᵥ1.4 (Santa Cruz; sc-32070). Whole-cell patch clamp recording and analysis were carried out on an Axopatch 200B amplifier with pClamp10 software (Axon Instruments). Patch electrodes were pulled from thin-walled borosilicate glass (World Precision Instruments) on a horizontal micropipette puller (Sutter Instruments). Electrodes had a resistance of 4-8 MΩ when filled with intracellular solution. Analog capacity compensation and 80% series resistance compensation were used during whole-cell recordings. For single pulse recordings, cells were depolarized to +10 mV from a holding potential of −80 mV at 10 s interval and P/4 leak subtraction procedure was used. Current density is presented after normalizing peak current amplitude to the corresponding cell capacitance value. To obtain the I-V relationship, a 200 ms ramp pulse protocol from −130 to 70 mV with −80 mV holding potential and P/4 leak subtraction procedure was used. Data were sampled at 50 kHz and filtered at 10 kHz and whole-cell recordings performed at room temperature (20° C.-22° C.). The extracellular solution contained 100 mM BaCl₂, 10 mM HEPES, adjusted to pH 7.4 with NaOH. The intracellular solution used in the pipettes contained 140 mM TEA-Cl, 5 mM EGTA, 10 mM HEPES, 1 mM MgATP₂, adjusted to pH 7.4 with TEA-OH.

Phospho-Flow Cytometric Signaling.

Thymocytes were incubated in HBSS with 10 mM HEPES for 30 min prior to stimulation. Cells were stimulated as above for the indicated time, fixed with 2% formaldehyde for 10 min, pelleted by centrifugation, and permeabilized overnight in 90% methanol at −20° C. For determination of STATS phosphorylation, permeabilized cells were treated with anti-STATS (pY649) mAb conjugated to AlexaFluor647 (BD Biosciences), anti-CD8α-PE, and anti-CD4-PE-Cy7 for 1 hr at room temperature. Flow cytometric measurements of ERK activity were performed as described (Priatel et al., 2002).

Immunoblotting.

To detect Caᵥ1.4, splenocytes were analyzed by immunoblot. Alternatively, T cells were isolated from splenocyte preparations with the EasySep Mouse T Cell Enrichment Kit (StemCell Technologies, Inc.). Membrane proteins were isolated and protein amounts between samples were normalized prior to immunoblotting as previously reported (Woodard et al., 2008). Binding of the primary Ab was detected with an Alexa 680-conjugated anti-rabbit IgG Ab (Li-Cor Biosciences). The protein bands were visualized with the Odyssey Infrared Imaging System (Li-Cor Biosciences). Signal intensities were quantified with Odyssey software. For signaling analysis, thymocytes were activated by TCR stimulation (as above) for the indicated time. As a positive control for activation, thymocytes were incubated with 100 ng/mL PMA for 10 min at 37° C. Ras activity was assessed as previously described (David et al., 2005). Phosphorylated and total ERK and JNK were detected by immunoblotting. The fold increase in phosphorylation was expressed as a ratio of total protein and was normalized to the unstimulated wild-type control.

NFAT Mobilization Assays.

Single-cell suspensions from thymi of WT or Cacnalf^{-/-} mice were prepared and incubated for 16 hr with plate-bound CD3ε (145-2C11) Ab (10 μg/ml) and soluble CD28 (1 μg/ml) or media alone. Whole cells were lysed for 10 min in RIPA buffer. Nuclear and cytoplasmic fractions were prepared with NE-PER Nuclear and Cytoplasmic Extraction Kit (Thermo Scientific) and analyzed by immunoblot. Binding of the primary Ab was detected as above. The fold increase in activation was expressed as a ratio of the appropriate loading control and was normalized to the unactivated wild-type control.

Naive T Cell Survival Assays.

WT (Thy1.1⁺) and Cacnalf^{-/-} (Thy1.2⁺) CD44^{lo}CD4⁺ and CD8⁺ T cells were sorted as described in electrophysiological assays described above. Purified WT and mutant naive CD4⁺ and CD8⁺ T cells were mixed at equivalent ratios (1:1:1:1) and 200,000 total cells per well were cultured in 96-well flat-bottom plates. Cells were treated either with the indicated dose of mIL-7 (eBioscience) or cultured in wells precoated with 10 μg/mL of CD3 (145-2C11) Ab. After 24 hr, viability was determined by labeling samples with CD8 and Thy1.1 Abs, incubating with Annexin V-Alexa 647 (Southern Biotech) in Ca²⁺-containing buffer for 15 min at RT, and subsequently acquiring data on a BD FACSCalibur.

Adoptive Transfer Experiments.

For naive T cell transfers, WT (Thy1.1⁺) and Cacnalf^{-/-} (Thy1.2⁺) CD44^{lo}CD4 and CD8 T cells were sorted as described in electrophysiological assays described above, mixed at a 1:1:1:1 ratio, fluorescently labeled with carboxyfluorescein succinimidyl ester (CFSE) (Invitrogen) and coinjected into Rag1^{-/-} hosts. 1 week posttransfer, splenocytes were isolated and stained with relevant Abs for discriminating donor WT and mutant T cells.

Bacterial Infections and the Detection of Antigen-Specific T Cells.

Mice were infected intravenously (i.v.) with ˜10⁴ colony forming units (CFU) of rLM-OVA (Listeria monocytogenes expressing ovalbumin). Splenocytes were stained with CD8α (53-6.7) and CD44 (IM7) Abs and H-2K^{b}-OVA tetramer (iTag MHC Tetramer, Beckman Coulter). Intracellular cytokine staining and cytotoxicity assays were performed as described (Priatel et al., 2007).

Statistical Analysis.

Statistical significance was determined with an unpaired Student's t test for most analyses. For electrophysiology assays, statistical significance was measured by the ANOVA test, with two-factorial design without replication.

### Results:

Caᵥ1.4 Deficiency Results in CD4⁺ and CD8⁺ T Cell Lymphopenia and Spontaneous T Cell Activation

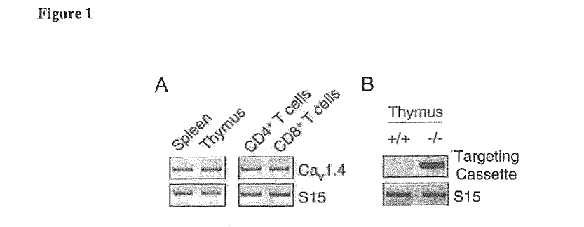

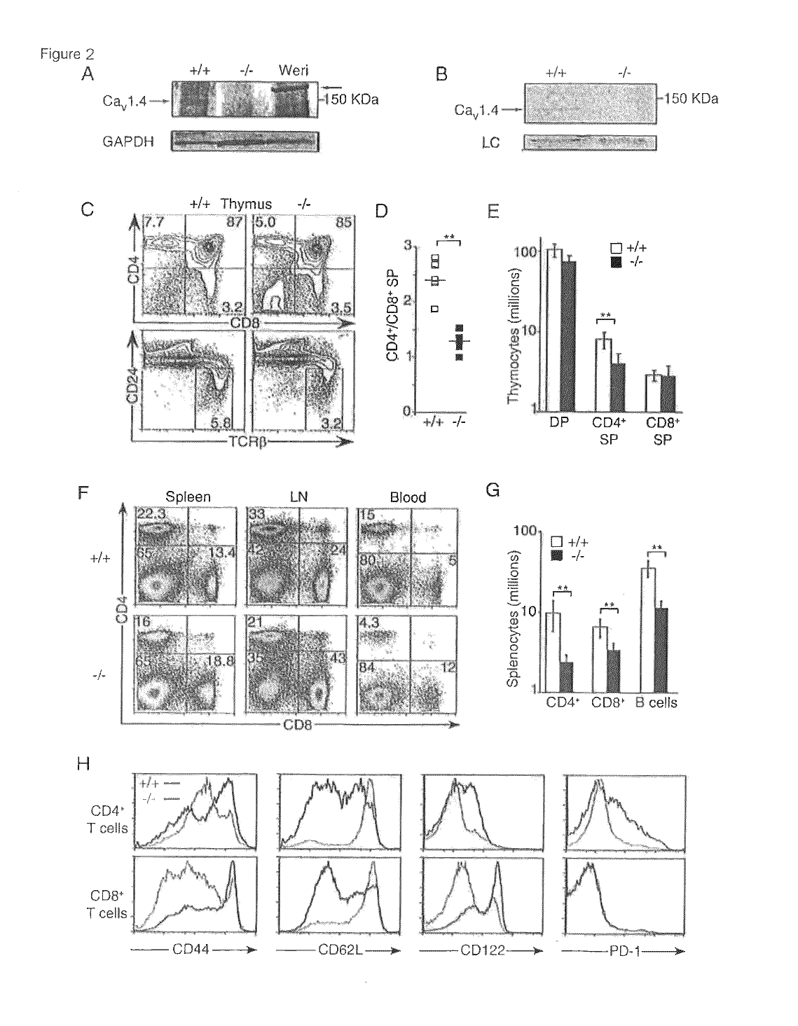

To characterize Caᵥ1.4 expression in wild-type (WT) mice, RNA analyses were performed and revealed expression in thymus, spleen, and peripheral CD4⁺ and CD8⁺ T cells (see FIG. 1A). Previous observations describing Caᵥ1.4 expression in developing and mature T cells led to the investigation of whether Cacnalf^{-/-} mice displayed a T cell phenotype. Cacnalf^{-/-} mice have been previously generated through gene targeting, inserting a stop codon and prematurely terminating Cacnalf translation (Mansergh et al., 2005). To verify gene targeting in Cacnalf^{-/-} mice, reverse transcriptase-polymerase chain reaction (RT-PCR) was performed, detecting the disrupted Caᵥ1.4 transcript carrying a loxP site specifically in the Cacnalf^{-/-} mice (FIG. 1B). In addition, Caᵥ1.4 antibody (Ab) blotting revealed protein loss among Cacnalf^{-/-} splenic whole cell lysates (FIG. 2A). Discrepancies in Caᵥ1.4 protein size between mouse splenocytes and Weri retinoblastoma cells may be a consequence of alternative splicing (Kotturi and Jefferies, 2005) or cell-type-specific posttranslational modifications. To establish whether Caᵥ1.4 is present at the T cell plasma membrane, WT and Cacnalf^{-/-} splenic T cells were surface biotinylated and streptavidin-coupled bead immunoprecipitates were blotted with Caᵥ1.4 Abs (FIG. 2B). The detection of Caᵥ1.4-size band specifically in WT T cells argues that Caᵥ1.4 channels are expressed at the T cell surface.

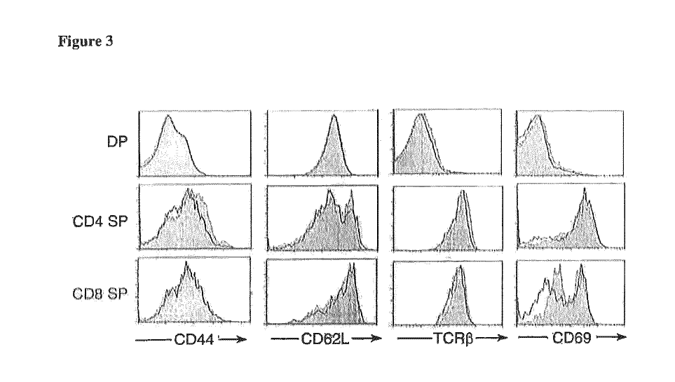

Analyses of thymocytes lacking a functional Caᵥ1.4 channel revealed a number of changes to T cell maturation. The ratio of CD4⁺ versus CD8⁺ single-positive (SP) thymocytes in Cacnalf^{-/-} thymi was skewed slightly toward the CD8⁺ lineage (FIG. 2C), and the proportion of mature thymocytes, distinguished as CD24^{lo}TCRβ^{hi}, was reduced relative to WT (FIG. 2D). The effect of Caᵥ1.4 deficiency on T cell development was also reflected in a 50% decrease in the number of mature CD4⁺ SP thymocytes whereas the number of CD8⁺ SP thymocytes was largely unchanged (FIG. 2E). However, the expression of various maturation and activation markers on Cacnalf^{-/-} double-positive (DP) and TCRβ⁺ SP subpopulations closely paralleled WT, expressing similar amounts of TCRβ, CD44, CD69, and CD62L (FIG. 3). Collectively, these findings suggest that Caᵥ1.4 functions promote positive selection, particularly differentiation of the CD4⁺ SP lineage.

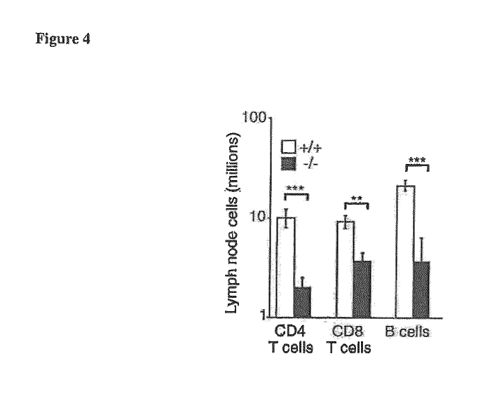

The examination of peripheral lymphoid compartments, including spleen, lymph nodes (LN), and peripheral blood, revealed that Cacnalf^{-/-} exhibited a decreased frequency of CD4⁺ T cells and a reduced ratio of CD4⁺ versus CD8⁺ T cells relative to WT mice (FIG. 2F). Furthermore, Cacnalf^{-/-} mice were found to be strikingly lymphopenic for CD4⁺ T cell, CD8⁺ T cell, and B cell subsets based on splenic (FIG. 2G) and LN (FIG. 4) cell recovery. Moreover, the loss of peripheral CD4⁺ T cells in Cacnalf^{-/-} mice was considerably more dramatic than for CD8⁺ T cells. Associated with the drop in Cacnalf^{-/-} T cell numbers, both CD4⁺ TCRβ⁺ and CD8⁺ TCRβ⁺ T cells showed signs of spontaneous acute T cell activation, expressing increased amounts of CD44, CD122, and programmed death (PD)-1 and reduced CD62L (FIG. 2H). In summary, these findings demonstrate that Caᵥ1.4-dependent Ca²⁺ signaling is essential for naive CD4⁺ and CD8⁺ T cell homeostasis and quiescence.

Caᵥ1.4 is Critically Required for TCR-Induced and Store-Operated Rises in Cytosolic-Free Ca²⁺

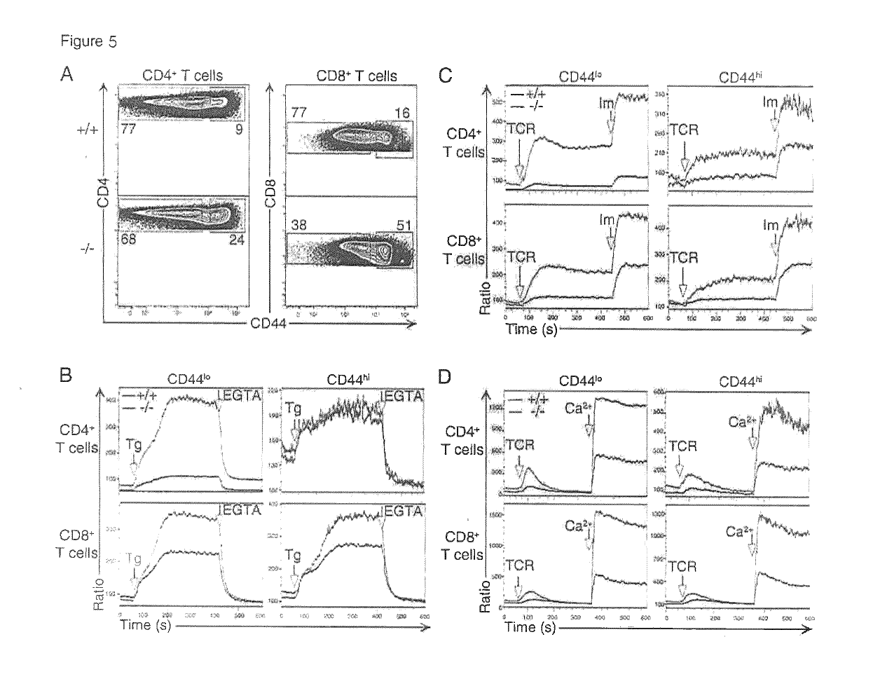

WT and Cacnalf^{-/-} splenocytes, loaded with indicator dyes for measuring cytosolic Ca²⁺ and labeled with CD4 and CD8 Abs plus CD44 Abs for the discrimination of CD44^{lo} (naive) or CD44^{hi} (memory) CD4⁺ and CD8⁺ T cell responses (FIG. 5A), were stimulated with the indicated agonists to investigate Ca²⁺ transport deficiencies in Cacnalf^{-/-} mice. To determine whether Ca²⁺ release from intracellular stores is competent for mediating Ca²⁺ influx via plasma membrane channels, splenic T cells were treated with thapsigargin (FIG. 5B). Thapsigargin, an inhibitor of a Ca²⁺-ATPase of the ER, induces rises in cytosolic Ca²⁺ concentration by blocking the cell's ability to pump Ca²⁺ into sarco- and endoplasmic reticula and secondarily activates plasma membrane-bound Ca²⁺ channels, triggering Ca²⁺ entry from outside the cell (Thastrup et al., 1990). Remarkably, Cacnalf^{-/-} CD44^{lo}CD4⁺ T cells exhibited greatly diminished increases in cytosolic Ca²⁺ upon thapsigargin stimulation whereas Cacnalf^{-/-} CD44^{lo} and CD44^{hi}CD8⁺ T cells also showed marked reductions relative to their WT counterparts (FIG. 5B). On the other hand, Ca²⁺ efflux from CD4⁺ and CD8⁺ T cells did not appear to be compromised by Caᵥ1.4 deficiency as demonstrated via addition of the Ca²⁺ chelator ethylene glycol tetraacetic acid (EGTA). In contrast to comparisons between naive CD4⁺ T cells, WT and Cacnalf^{-/-} CD44^{hi}CD4⁺ T cells displayed very similar Ca²⁺ responses. Together, these observations demonstrate that Caᵥ1.4 channels are critically required for SOCE in CD44^{lo}CD4⁺ T cells and to a lesser extent in CD44^{lo} and CD44^{hi}CD8⁺ T cells.

To investigate whether Caᵥ1.4 channels might regulate TCR signaling, WT and mutant splenocytes, precoated with biotinylated CD3 Abs, were activated by streptavidin (SA) addition. In WT T cells, TCR crosslinking induced cytosolic Ca²⁺ concentrations to rise rapidly and remain elevated for sustained duration (FIG. 5C). Paradoxically to the responses observed for thapsigargin treatment, both Cacnalf^{-/-} CD4⁺ and CD8⁺ T cells responded very weakly to TCR stimulus regardless of their surface CD44 phenotype. The basis for differential CD4⁺ and CD8⁺ T cell dependence on Caᵥ1.4 function for thapsigargin but not TCR responses is unclear (FIG. 5B). In addition, Cacnalf^{-/-} T cells, particularly CD44^{lo}CD4⁺ T cell subset, reached greatly reduced peak Ca²⁺ concentrations relative to WT upon treatment with ionomycin. Ionomycin increases cytosolic Ca²⁺ concentrations via its ionophoric properties, releasing intracellular Ca²⁺ stores and subsequently stimulating the opening of plasma membrane Ca²⁺ channels and Ca²⁺ influx from outside the cell (Morgan and Jacob, 1994). The findings that ionomycin responses were greatly blunted in Cacnalf^{-/-} T cells suggests that Caᵥ1.4 function contributes to the storage of intracellular Ca²⁺ or is critical for the importation of Ca²⁺ after its release from intracellular stores.

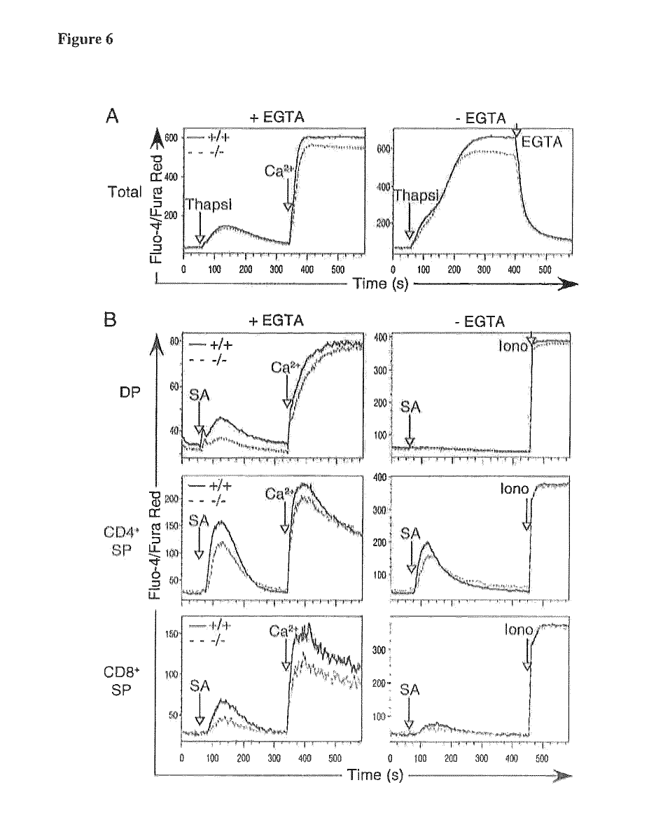

To determine whether Caᵥ1.4 mediates one or both of the aforementioned processes involved in Ca²⁺ responses, Ca²⁺ responses were monitored after TCR stimulation when extracellular Ca²⁺ was chelated by EGTA, preventing Ca²⁺ intake and thereby uncovering Ca²⁺ release from intracellular stores. The transient cytosolic Ca²⁺ elevation observed after TCR ligation in the presence of EGTA was found to be decreased in Cacnalf^{-/-} T cells relative to WT (FIG. 5D). Furthermore, the repletion of extracellular Ca²⁺, facilitating Ca²⁺ influx across the plasma membrane, resulted in a dramatic cytosolic Ca²⁺ surge in WT T cells whereas increases by Cacnalf^{-/-} T cells were markedly less. In addition, it was found that Caᵥ1.4 also functions in thymocytes and was important for rises in cytosolic Ca²⁺ when TCR stimulations were performed in the absence of extracellular Ca²⁺ (FIG. 6).

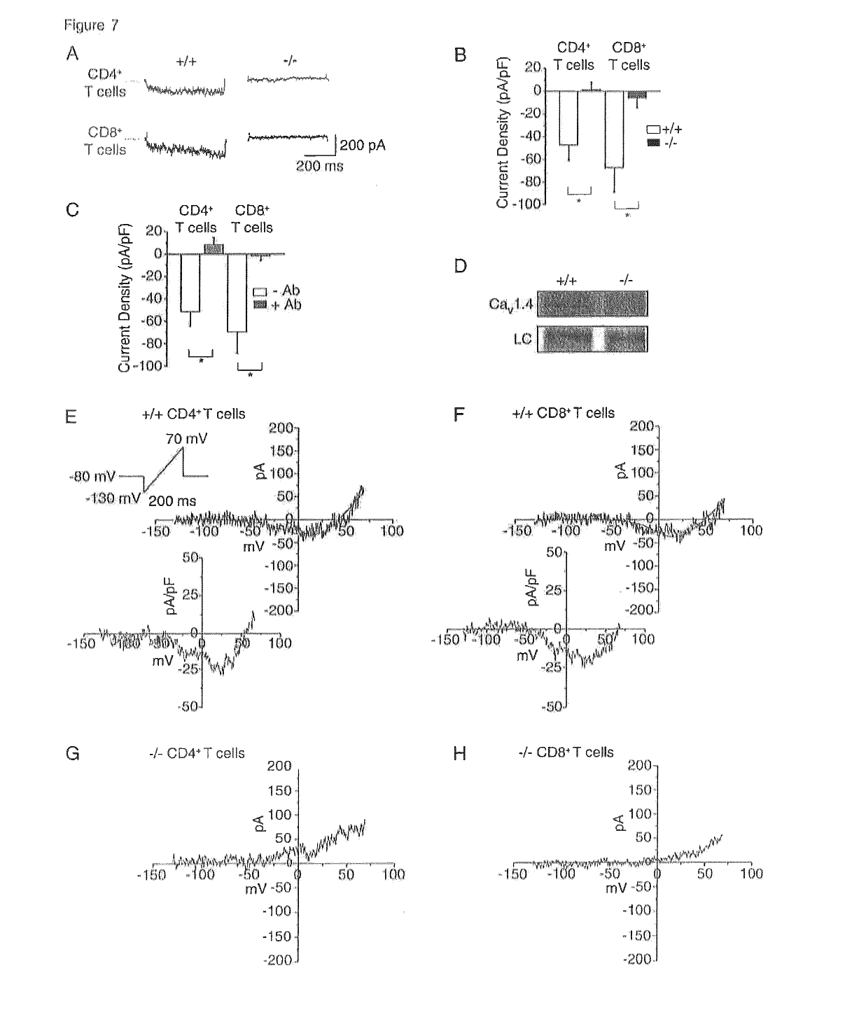

To verify that Caᵥ1.4 regulates Ca²⁺ entry into the cell, the channel current was monitored after TCR stimulation by employing barium (Ba²⁺) as a carrier ion in patch clamp experiments. Ba²⁺ used as a Ca²⁺ mimic provides a number of key benefits because it augments currents, by (1) having a higher conductance through Ca²⁺ channels, (2) blocking potassium channels efficiently, and (3) decreasing secondary signal transduction associated with Ca²⁺ influx. Ca²⁺ current in Cacnalf^{+/+} and Cacnalf^{-/-} CD44^{lo}CD4⁺ and CD8⁺ T cells was characterized with a single sweep protocol from −80 mV to +10 mV. Currents were detected in WT but not mutant T cells after TCR cross-linking (FIGS. 7A and 7B). To determine whether L-type channels are functioning at the plasma membrane, TCR-induced inward currents were compared in presence or absence of an ectodomain-specific Caᵥ1 al subunit Ab. The addition of the Ab to WT CD44^{lo}CD4⁺ and CD8⁺ T cells was found to block inward currents observed after TCR stimulation (FIG. 7C). Furthermore, treatment with control goat Abs did not reveal any effects on inward currents. To verify that the ectodomain Caᵥ1 α1 subunit Ab recognizes Caᵥ1.4, WT and Cacnalf^{-/-} splenocyte extracts were incubated with the ectodomain Caᵥ1 α1 subunit Ab and immunoprecipitates were blotted with a Caᵥ1.4 Ab (FIG. 7D). The detection of a Caᵥ1.4 band specifically in WT but not Cacnalf^{-/-} cells supports the conclusion that Caᵥ1.4 acts as a conduit for the influx of Ca²⁺ upon TCR ligation.

To further characterize the type of TCR-induced Ca²⁺ currents in WT and Cacnalf^{-/-} T cells, a ramp pulse protocol was used to measure I-V curves upon TCR cross-linking (FIGS. 7E and 7F). The peak voltages of I-V relationships (Vₘₐₓ) were 16.3±5.2 mV (n=5) and 24.4±3.3 mV (n=5) for WT CD44^{lo}CD4⁺ and CD8⁺ T cells, respectively. The half activation potentials (Vₐ), which were obtained from the modified Boltzmann fits, were −0.2±4.7 mV (n=5) for CD4⁺ T cells and 1.3±3.5 mV (n=5) for CD8⁺ T cells. Those Vₐ values were comparable with previous reports examining the characteristics of the L-type Caᵥ1.4 channel expressed in heterologous systems (Baumann et al., 2004; McRory et al., 2004). By contrast, Cacnalf^{-/-} CD4⁺ and CD8⁺ T cells did not show any inward current in response to the ramp pulse (FIGS. 7G and 7H). Collectively, these data suggest that Caᵥ1.4 is operated by TCR signaling and that it may serve to replenish intracellular Ca²⁺ stores in developing and naive T cells.

### Caᵥ1.4 Function Regulates Ras-ERK Activation and NFAT Mobilization

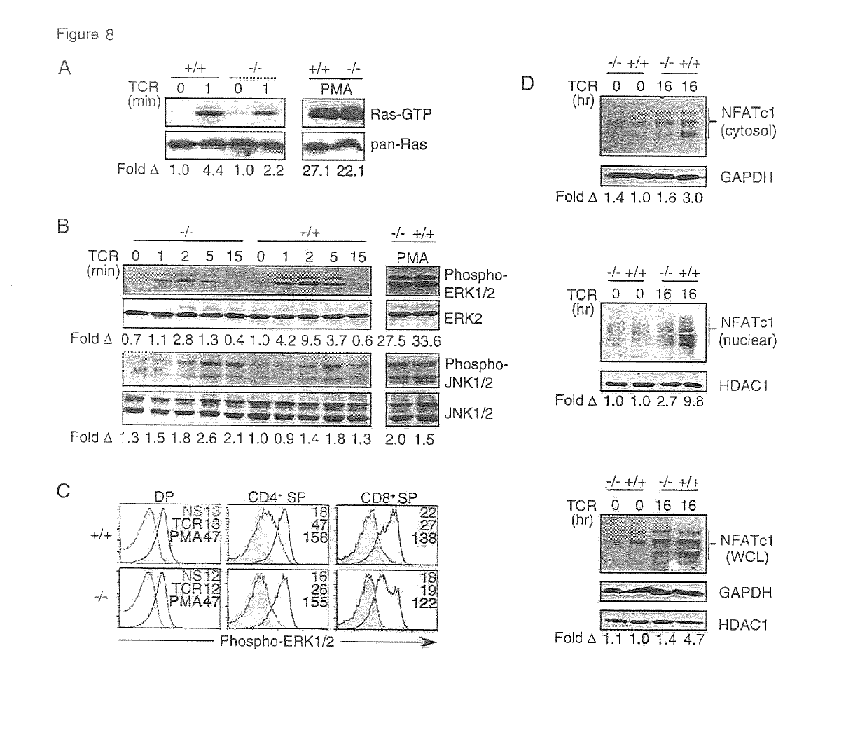

To address whether Caᵥ1.4 channels affect Ras-MAPK signaling, a pathway heavily implicated in controlling T cell survival and differentiation (Alberola-Ila and Hernández-Hoyos, 2003), studies were initiated to measure the activation status of these downstream effectors after TCR stimulation. For Ras signaling, WT and Cacnalf^{-/-} thymocytes were stimulated with TCR Ab and subsequently Ras activation was assessed by precipitation of Ras-GTP with Raf-1-GST fusion protein (FIG. 8A). Cacnalf^{-/-} thymocytes were found to induce 50% less Ras-GTP as compared to wild-type cells. By contrast, the amount of activated Ras was fairly comparable between genotypes when cells were stimulated with the diaceyl glycerol (DAG) analog PMA. Next, an analysis of the activation of downstream-acting MAP kinases ERK and JNK in total thymocytes at the indicated times post-TCR stimulation was performed (FIG. 8B). The intensity and duration of ERK activation after TCR crosslinking was reduced in Cacnalf^{-/-} thymocytes relative to WT. However, comparison of JNK phosphorylation between WT and Cacnalf^{-/-} thymocytes upon TCR stimulation revealed only marginal differences. By contrast, PMA treatment was found to induce strong ERK and JNK phosphorylation regardless of cell genotype. Collectively, these studies reveal that Caᵥ1.4 deficiency selectively affects the activation of ERK. To assess whether ERK activation is affected in Cacnalf^{-/-} mature SP thymocytes, ERK activity was assessed with phospho-flow cytometry before and after stimulation with TCR Abs or PMA treatment (FIG. 8C). Cacnalf^{-/-} CD4⁺ and CD8⁺ SP thymocytes exhibited reduced ERK activation relative to WT upon TCR but not PMA stimulation.

NFAT proteins, critical regulators of thymocyte development and T cell differentiation, are phosphorylated and reside primarily in the cytoplasm of resting T cells (Oh-hora, 2009). Upon T cell receptor stimulation, Ca²⁺ signals induce the activation of the serine-threonine phosphatase calcineurin, catalyzing NFAT dephosphorylation and triggering its subsequent translocation to the nucleus. To determine whether deficient Ca²⁺ release after TCR ligation affected NFAT translocation and activation in Cacnalf^{-/-} thymocytes, NFATc1 amounts in the cytosolic and nuclear fractions of WT and Cacnalf^{-/-} thymocytes were examined (FIG. 8D). Cacnalf^{-/-} thymocytes were found to have less nuclear NFATc1 as compared to WT cells. Together, these experiments demonstrate that Caᵥ1.4-dependent Ca²⁺ entry regulates the activation of the NFAT and ERK pathways.

### T Cell-Intrinsic Caᵥ1.4 Function is Required for Normal T Cell Homeostasis

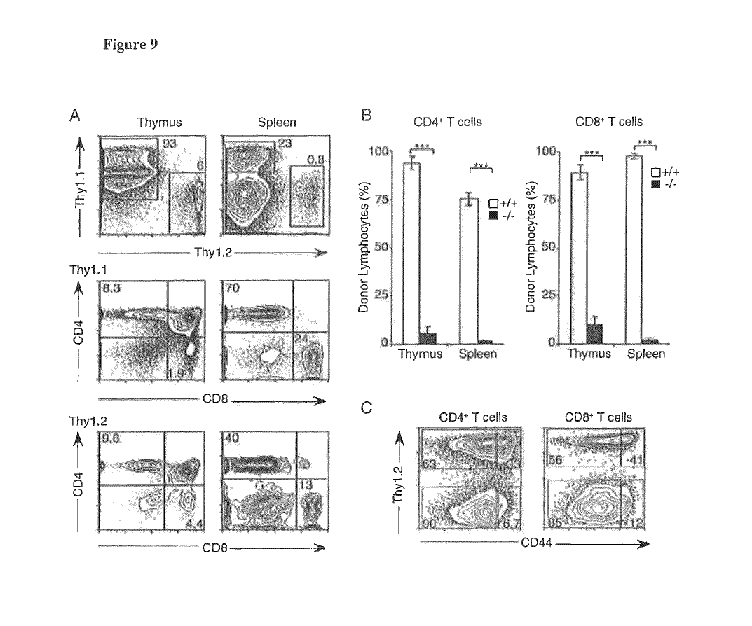

To determine whether the loss of Caᵥ1.4 function in T cells themselves contributes to the impaired T cell development and/or peripheral T cell maintenance, bone marrow transfer experiments were performed in which equivalent numbers of T cell-depleted WT (Thy1.1⁺Ly5.2⁺) and Cacnalf^{-/-} (Thy1.2⁺Ly5.2⁺) bone marrow was transferred into irradiated congenic (Ly5.1⁺) hosts. After 1 month posttransfer, evaluation of donor cell frequencies (Ly5.2⁺) in the thymus and spleen revealed that Cacnalf^{-/-} bone marrow cells competed very poorly with WT for T cell reconstitution of the host (FIG. 9A). The frequency of WT donor CD4⁺ and CD8⁺ T cells in the thymus and periphery was substantially higher than that of the Cacnalf^{-/-} CD4⁺ and CD8⁺ T cells, respectively (FIGS. 9A and 9B). Furthermore, comparison of the ratio of CD44^{lo} versus CD44^{hi}CD4⁺ and CD8⁺ T cells populations showed that Cacnalf^{-/-} splenic donor T cells were skewed toward a memory phenotype relative to wild-type donor T cells (FIG. 9C). Moreover, these experiments suggest that the heightened frequency of Cacnalf^{-/-} CD44^{hi} T cells in Cacnalf^{-/-} mice is not a consequence of lymphopenia but rather due to a failure of Cacnalf^{-/-} CD44^{lo} T cells to be maintained. Together, these results demonstrate a cell-intrinsic function of Caᵥ1.4 in T cell progenitors and/or mature T cells that is necessary for efficient T cell reconstitution.

### Caᵥ1.4 is an Important Regulator of Naive T Cell Homeostasis

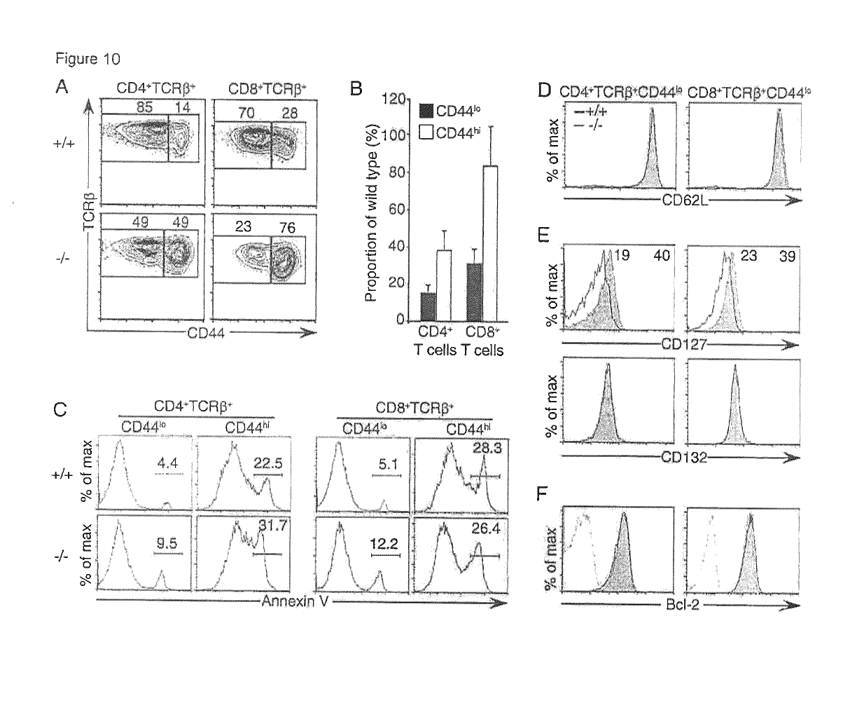

The finding that Cacnalf^{-/-} mice are lymphopenic and that a majority of the residual T cells possess an activated or memory phenotype suggested that Caᵥ1.4 functions are essential for naive T cell maintenance. Moreover, comparison of T cell subsets based on CD44 expression revealed that Cacnalf^{-/-} mice exhibited a severe loss of CD44^{lo} T cells relative to WT whereas CD44^{hi} T cell numbers were much less affected (FIGS. 10A and 10B). To determine whether cell turnover rates differed between cohorts, WT and Cacnalf^{-/-} T cells were stained with the apoptotic marker Annexin V (FIG. 10C). Cacnalf^{-/-} CD44^{lo} but not CD44^{hi} T cells displayed enhanced Annexin V reactivity relative to their WT counterparts. Surface phenotypic examination of Cacnalf^{-/-} CD44^{lo} T cells showed that they seemed mature, resembling WT naive T cells with respect to CD62L, TCRβ, and CD69 expression (see, for example, FIG. 10D). Together, these data suggest that the limited number of CD44^{lo} T cells in Cacnalf^{-/-} mice is at least in part a consequence of their decreased fitness.

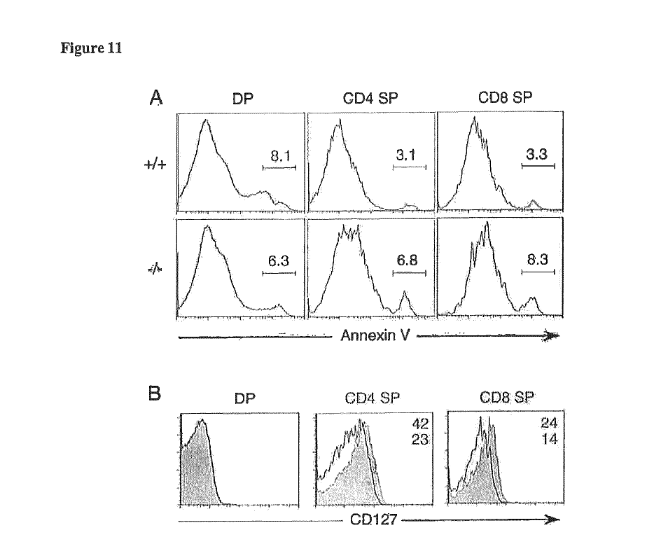

Signaling through the IL-7 receptor (IL-7R), a heterodimer of IL-7Rα (CD127), and the common γ-chain (CD132) plays a governing role in naive T cell homeostasis, and loss of either IL-7 or IL-7R in both mice and humans results in T cell lymphopenia and severe immunodeficiency (Surh and Sprent, 2008). Therefore, IL-7R expression in Cacnalf^{-/-} CD44^{lo} T cells was investigated (FIG. 10E). Cacnalf^{-/-} CD44^{lo} T cells were found to express only about 50% of WT CD127 amounts but WT CD132 expression. Analyses of Annexin V reactivity and IL-7R between WT and Cacnalf^{-/-} CD4⁺ and CD8⁺ TCRβ⁺ SP thymocytes revealed similar findings as noted above for comparisons of peripheral CD44^{lo} T cells (FIG. 11). Despite reduced CD127 expression, Cacnalf^{-/-} CD44^{lo}CD4⁺ and CD8⁺ T cells displayed WT amounts of the prosurvival protein Bcl-2 (FIG. 10F). These findings suggest that Caᵥ1.4 may affect naive T cell fitness in part through CD127 regulation.

### Caᵥ1.4 Promotes Survival Signaling and Homeostasis-Induced T Cell Expansion

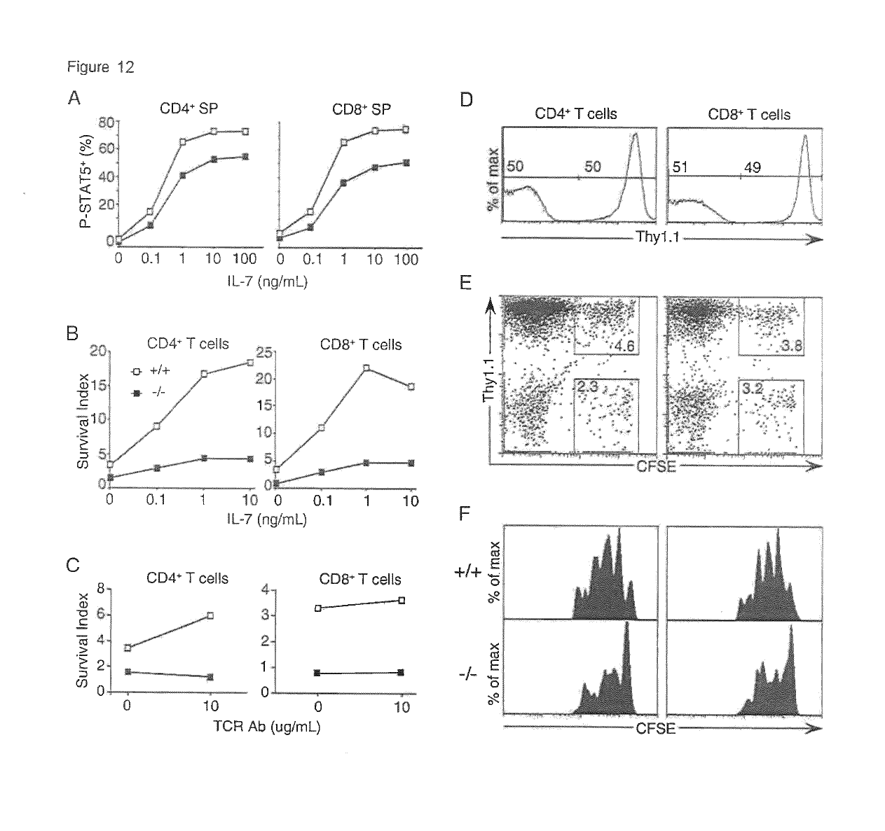

To determine whether Caᵥ1.4 deficiency and its concomitant reduction in IL-7Rα expression is functionally significant, IL-7R signaling was monitored by tracking the phosphorylation status of its downstream effector STATS (FIG. 12A). WT and Cacnalf^{-/-}CD4⁺ and CD8⁺ SP thymocytes were stimulated with various doses of IL-7 and stained with a phospho-Y647 STATS-specific Ab. Cacnalf^{-/-} CD4⁺ and CD8⁺ SP thymocytes showed a marked reduction in STATS phosphorylation as compared to WT at all IL-7 doses tested. Next, whether Caᵥ1.4 deficiency affects the capacity of IL-7 to promote T cell survival was investigated. WT and Cacnalf^{-/-} CD44 T cells were isolated by cell sorting and placed into culture with the indicated concentrations of IL-7, and their viability was assessed 24 hr later via Annexin V staining (FIG. 12B). Cacnalf^{-/-} CD44^{lo} T cells were found to be much less capable than WT at utilizing IL-7 to support their survival in vitro. In addition, Cacnalf^{-/-} CD44^{lo}CD4⁺ T cells exhibited reduced survival relative to WT when placed into TCR Ab-coated wells for 24 hr ex vivo culture (FIG. 12C). Collectively, these findings suggest that Caᵥ1.4 channel protein impacts naive T cell survival through the regulation of either IL-7 or TCR signaling.

The size of the naive T cell compartment is restrained by the availability of both IL-7 and self peptides-major histocompatibility complex (MHC) molecules (Surh and Sprent, 2008). To examine the proliferative potential of Cacnalf^{-/-} CD44^{lo} T cells in vivo, WT (Thy1.1⁺) and Cacnalf^{-/-} (Thy1.2⁺) CD44^{lo}CD4⁺ and CD8⁺ T cells were purified, mixed together at a 1:1:1:1 ratio, labeled with carboxyfluorescein diacetate succinimidyl ester (CFSE), and injected into congenitally lymphopenic Rag1^{-/-} hosts (FIG. 12D). After residing for 7 days in vivo, donor T cells were recovered and their cellular proliferation was assessed via CFSE dilution (FIG. 12E). By using the congenic marker Thy1.1, it was found that the proportion of donor WT cells recovered was considerably greater than Cacnalf^{-/-} cells. By electronically gating on donor T cells probably responding to cues from IL-7 and self-peptides-MHC molecules (Kieper et al., 2005), Cacnalf^{-/-} CD4⁺ and CD8⁺ T cells were found to have undergone fewer cell divisions than WT CD4⁺ and CD8⁺ T cells (FIG. 12F). Collectively, these studies suggest that a cell-intrinsic Caᵥ1.4 function is critical for T cells to respond appropriately to homeostatic and survival cues.

Caᵥ1.4 Functions are Necessary for Functional CD4⁺ and CD8⁺ T Cell Immune Responses

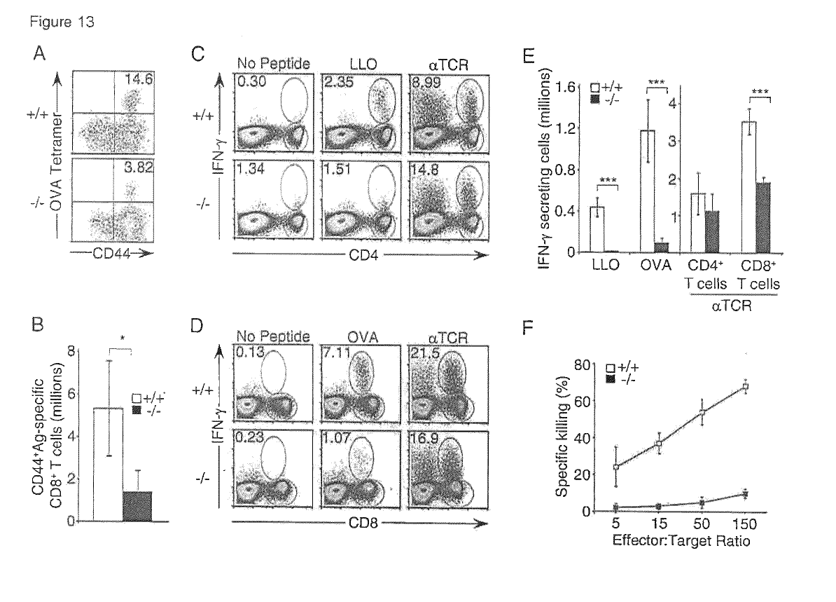

To investigate the requirement of Caᵥ1.4 function in an immune response, WT and Cacnalf^{-/-} mice were challenged with a recombinant Listeria monocytogenes expressing ovalbumin (rLM-OVA). Cacnalf^{-/-} mice produced substantially decreased numbers of OVA-reactive CD8⁺ T cells upon challenge with rLM-OVA (FIGS. 13A and 13B). Numbers of functional antigen-specific CD4⁺ and CD8⁺ T cells were drastically reduced in Cacnalf^{-/-} mice relative to WT (FIGS. 13C and 13D). In addition, the total numbers of IFN-γ-producing CD8⁺ T cell effectors were also diminished in Cacnalf^{-/-} mice (FIG. 13E). Next, the cytolytic function of purified CD8⁺ T cells from rLM-OVA-infected WT and Cacnalf^{-/-} mice was evaluated (FIG. 13F). Cacnalf^{-/-} mice exhibited a greatly weakened capacity to generate antigen-specific CTLs relative to WT. Together, these studies show that Caᵥ1.4 is critical for mounting productive CD4⁺ and CD8⁺ T cell responses.

### DISCUSSION

Caᵥ channels are major passageways controlling Ca²⁺ entry in excitable cells and regulate numerous processes including muscle contraction, neuronal signal transmission, and gene transcription (Feske, 2007). However, the biological roles of Caᵥ channels in nonexcitable cells such as lymphocytes are poorly defined. Identification of a mutation in the β4 subunit of VDCCs underlying the neurologic and immune system defects observed in the lethargic mouse line implicated Caᵥ function in immunoregulation (Burgess et al., 1997). In addition, a manuscript describing mice deficient in the β3 regulatory subunit has argued that Caᵥ channels play a role in modulating TCR signaling and CD8⁺ T cell homeostasis (Jha et al., 2009). To investigate the physiological functions of the L-type Caᵥ1.4 channel in developing and mature T cells, mice deficient in its pore-forming α1 subunit were analyzed. The studies described in this Example indicate that Caᵥ1.4 channels are critical for both the survival of naive CD4⁺ and CD8⁺ T cells and the generation of pathogen-specific CD4⁺ and CD8⁺ T cell responses. In addition, naive CD4⁺ and CD8⁺ T cells were shown to be dependent on Caᵥ1.4 function for SOCE, TCR-induced rises in cytosolic Ca²⁺ and downstream TCR signal transduction.

Analyses of Cacnalf^{-/-} mice revealed that T cells of various stages of development and differentiation showed differing relative dependence on Caᵥ1.4 for mediating Ca²⁺ responses. For instance, Cacnalf^{-/-} SP thymocytes exhibited more moderate decreases in TCR- or thapsigargin-induced rises in cytosolic-free Ca²⁺ relative to WT than what was observed when peripheral naive and memory WT and Cacnalf^{-/-} T cells were compared.

Caᵥ1.4 channels may regulate the Ras-ERK cascade through effects on RasGRP1, a Ras-guanyl nucleotide exchange factor. RasGRP1's two “EF hand” domains function by binding Ca²⁺, dictating its cellular localization and the duration of Ras-ERK signaling (Teixeiro and Daniels, 2010). In addition, the finding that the loss of Caᵥ1.4 influences TCR signal transduction suggests that central or peripheral tolerance could be impaired in Cacnalf^{-/-} mice. Although negative selection studies with Cacnalf^{-/-} TCR transgenic mice have not been performed, the numbers of splenic regulatory T (Treg) cell, defined as CD4⁺CD25⁺FoxP3⁺ cells, in Cacnalf^{-/-} mice was 50% of the Treg cells in WT (Cacnalf^{-/-}=0.84±0.23×10⁶ versus WT=1.75±0.44×10⁶). However, it is likely that neither the deletion of autoreactive T cells in the thymus nor their suppression by regulatory T cells in the periphery is perturbed by Caᵥ1.4 deficiency because old Cacnalf^{-/-} mice, bred 13 generations onto a C57BL/6 background, appear healthy, lacking any gross histological abnormalities among various tissues examined, and remain lymphopenic.

The finding that Caᵥ1.4 is critical for naive CD4⁺ and CD8⁺ T cell homeostasis suggests that this channel modulates signals required for their survival: TCR signaling upon contact with self peptides-MHC molecules and IL-7R signaling after IL-7 exposure (Surh and Sprent, 2005). Previous work has suggested that naive T cell TCR recognition of MHC molecules on dendritic cells triggers small Ca²⁺ responses that are necessary for their survival (Revy et al., 2001). As a result, we hypothesize that low-affinity TCR interactions with self-antigens induce naive T cells to open Caᵥ1.4 channels perhaps as a direct consequence of TCR signaling or through an interaction with STIM1 (Park et al., 2010; Wang et al., 2010). Notably, Caᵥ1.4, as well as Caᵥ1.3, has been found to have low activation thresholds that do not require strong depolarizations for their activation (Lipscombe et al., 2004). Caᵥ1.4-mediated influx of Ca²⁺ from outside the cell probably induces a signaling cascade as well as contributes to tonic filling of intracellular Ca²⁺ stores critical for TCR survival signaling. We suspect that at least two secondary factors may contribute to the Ca²⁺ release defects observed by Cacnalf^{-/-} T cells upon stimulation: (1) decreased ER Ca²⁺ stores resulting in reduced SOCE and (2) diminished inward Ca²⁺ flux through CRAC channels collaborating to impair Ca²⁺-dependent signaling. Notably, low-grade TCR signaling and naive T cell homeostasis have been shown to be dependent on RasGRP1 (Priatel et al., 2002). Together, these data suggest that the Ca²⁺ current controlled by lymphoid-expressed Caᵥ1.4 channels influence the viability of naive T cells and may be essential for preserving a naive T cell population that expresses a diverse repertoire of TCRs.

### Example 2

### Inhibition of Caᵥ1 with a Blocking Antibody Reduces Survival of CD8⁺ and CD4⁺ T Cells

### T Cell Survival Assay

C57Bl/6 splenocytes were cultured in a 96-well flatbottom plate at 5×10⁶ cells/well in RPMI completed media with or without an ectodomain-specific Caᵥ1 α1 subunit antibody (clone SC-32070; Santa Cruz). This antibody was generated against Caᵥ1.3 but cross reacts with Caᵥ1.4. As shown in FIG. 7D, this antibody binds Caᵥ1.4 in splenocytes.

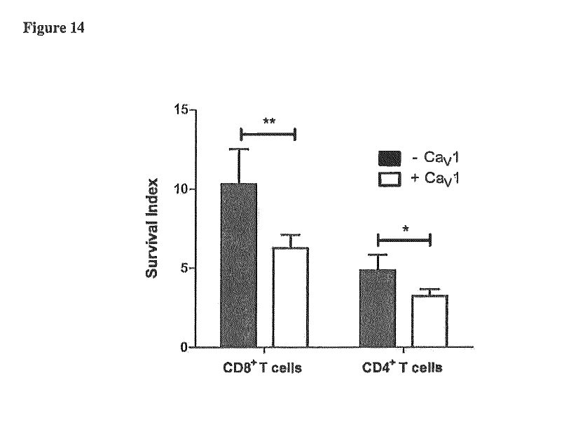

After 24 hrs, viability was determined by labeling samples with CD8 (clone 53-6.7; BD Biosciences), and CD4 (clone GK1.5; BD Biosciences) antibodies, incubating with Annexin V-Alexa 647 (Invitrogen) in Ca²⁺-containing buffer for 15 min at RT, and subsequently acquiring data on a BD FACSCalibur. The results of this experiment are provided in FIG. 14.

As described in Example 1, CD4⁺ and CD8⁺ T cells that lack Caᵥ1.4 protein exhibit reduced survival in the periphery. To verify that inhibition of Caᵥ1.4 function results in decreased T cell fitness, splenocytes were incubated with or without an ectodomain-specific Caᵥ1 α1 subunit antibody. As shown in FIG. 14, in the presence of the blocking antibody, CD4⁺ and CD8⁺ T cells displayed enhanced Annexin V reactivity indicating increased apoptosis. This Example therefore confirms that the Caᵥ1.4 channel contributes to naive T cell maintenance and inhibition of Caᵥ1.4 function with a blocking antibody impairs T cell survival.

### Example 3

### Inhibition of Caᵥ1 with a Blocking Antibody Reduces CD8⁺ and CD4⁺ T Cell Proliferation

### T Cell Proliferation Assay

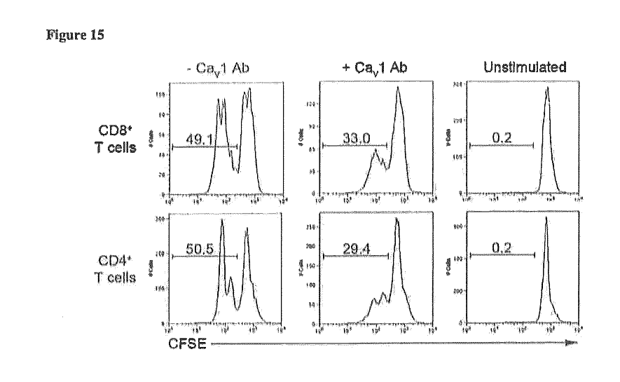

C57Bl/6 splenocytes were CFSE (Invitrogen) labeled and cultured in a 96-well flatbottom plate at 5×10⁶ cells/well in RPMI completed media with or without Caᵥ1 Ab (clone SC-32070; Santa Cruz). Cells were activated with 10 μg/mL of plate-bound CD3ε (clone 145-2C11) and 5 μg/ml of plate bound CD28 (clone 37.51) antibodies. After 5 days, samples were labeled with CD8 (clone 53-6.7; BD Biosciences), and CD4 (clone GK1.5; BD Biosciences) antibodies and T cell proliferation was assessed by CFSE dilution using the BD FACSCalibur. The results of this experiment are provided in FIG. 15.

As shown in Example 1, absence of Caᵥ1.4 protein diminished CD4⁺ and CD8⁺ T cell proliferative potential. To confirm the inhibition of cell surface Caᵥ1.4 affects T cell division, splenocytes labeled with CFSE were activated through the TCR with plate-bound CD3 and soluble CD28 antibodies and incubated with or without an ectodomain-specific Caᵥ1 al subunit antibody. As shown in FIG. 15, in the presence of the blocking antibody, CD4⁺ and CD8⁺ T cells were found to have undergone fewer cell divisions. This Example demonstrates that inhibition Caᵥ1.4 function with a blocking antibody reduces T cell proliferation following TCR activation.

### Example 4

### Role of Caᵥ1.4 Calcium Channel in B Lymphocytes

The following experiments were carried out to determine the physiological functions of Caᵥ1.4 in B cell biology.

### Experimental Methods:

Mice: Cacnalf−/− mice have been previously described (Mansergh et al., 2005). These mice were backcrossed to the C57BL/6 (CD45.2+) background for at least 13 generations. B6.SJL-Ptprca Pep3b/BoyJ (CD45.1+) mice were obtained from The Jackson Laboratory (Bar Harbor, Me.). Studies were performed according to guidelines set by the Canadian Council on Animal Care and the Animal Care Committee of the University of British Columbia.

Flow Cytometry:

For analysis of B cell development, single-cell suspensions of bone marrow, spleen and peritoneal cavity lavages were prepared and following erythrocyte lysis, cells were stained for 30 min on ice with various antibodies to cell surface makers used to identify specific B cell subsets as indicated in the figures. To assess the surface expression of BAFF receptor, splenocytes were surface stained with BAFF receptor, B220, IgM, CD21 and CD23 antibodies. Data were acquired using a BD™ LSR II flow cytometer (BD Biosciences) with FACSDiva™ software and analyzed with FlowJo software (Treestar).

Splenic B Cell Purification and In Vitro Stimulation:

For primary murine B cell purification, single-cell suspensions were prepared from spleens of wild-type C57BL/6 or Cacnalf−/− mice. Following erythrocyte lysis, B lymphocytes were negatively selected using the EasySep Mouse B cell Enrichment Kit (STEMCELL Technologie) according to the manufacturer's instructions. Purified splenic B lymphocytes, which were typically >90% B220+ by flow cytometry analysis, were then resuspended in RPMI 1640 (Invitrogen) supplemented with 10% FBS, 2 mM L-glutamine, 50 μM β-mercaptoethanol, 10 mM HEPES, and 100 U/mL penicillin, 100 μg/ml streptomycin. To assess the level of expression of surface activation markers upon stimulation, splenic B cells were left unstimulated or stimulated with F(ab′)2 fragment goat anti-mouse IgM (Jackson ImmunoResearch), anti-mouse CD40 (eBiosciences) or lipopolysaccharide (LPS, Invivogen) at the indicated concentrations. Twenty-four hours later, cells were stained with B220, CD69 and CD86 antibodies and analyzed by flow cytometry.

For the proliferation assay, purified B lymphocytes were labeled with 2 uM carboxyfluorescein diacetate succinimidyl ester (CFSE, Molecular Probes) and cultured in the presence or absence of anti-IgM or LPS at the indicated concentrations. After 72 h, CFSE dilution was analyzed by flow cytometry.

To assess the survival of B cells upon BAFF stimulation, purified splenic B cells were cultured in the presence or absence of recombinant mouse BAFF (R&D Systems) at the indicated concentrations for 72 h. The percentage of live cells was assessed by flow cytometry following staining with propidium iodide (Molecular probes). Data were acquired using a BD™ LSR II flow cytometer (BD Biosciences) with FACSDiva™ software and analyzed with FlowJo software (Treestar).

Bone Marrow Chimeras:

Donor bone marrow from CD45.2+ wild-type or Cacnalf−/− mice were mixed with competitor bone marrow from CD45.1+CD45.2+ congenic wild-type mice at a ratio of 1:1. A total of 3×10⁶ bone marrow cells per mouse were intravenously injected into recipient CD45.1+ wild-type mice subjected to 1,100 rads of gama-irradiation. Eight weeks after reconstitution, spleen, bone marrow and peritoneal cavity cells were collected for analysis.

Cytoplasmic and Mitochondrial Ca²⁺ Measurements:

To investigate the participation of Caᵥ1.4 in B cell Ca²⁺ flux, splenocytes from wild-type C57BL/6 or Cacnalf−/− mice were loaded with the intracellular calcium dyes Fluo-4 and FuraRed (Molecular Probes) in HBSS containing 2% FBS for 45 min at room temperature. Following washing, cells were surface stained with B220 antibody for 30 min on ice. Samples were suspended in RPMI and prewarmed for 15 min at 37° C. prior to stimulation. Cells were stimulated with 30 μg/mL of F(ab′)2 fragment goat anti-mouse IgM (Jackson ImmunoResearch), 1 μM of thapsigargin (Molecular Probes) or 1 μg/mL of ionomycin (Sigma) at the indicated time points. Chelation of extracellular Ca²⁺ was carried out by addition of ethylene glycol tetraacetic acid (EGTA). The intracellular Ca²⁺ levels in splenic B lymphocytes (B220+) were plotted as the ratio of Fluo-4/FuraRed over time. To assess whether Caᵥ1.4-deficiency results in alterations in mitochondrial calcium uptake, splenocytes from wild-type C57BL/6 or Cacnalf−/− mice were be loaded with Rhod-2 (Molecular Probes), a mitochondrial Ca²⁺ indicator and analyzed by flow cytometry. Rhod-2 was reduced to dihydrorhod-2 before loading into cells which has been shown to improve the discrimination between cytosolic and mitochondrially localized dye. Rhod-2 labeled cells were then stained with B220 antibody and stimulated as indicated above in the presence or absence of carbonyl cyanide 3-chlorophenylhydrazone (CCCP, Molecular Probes) to disrupt the mitochondrial membrane potential or EGTA to chelate extracellular Ca2⁺. Parallel experiments were conducted in splenocytes from wild-type C57BL/6 or Cacnalf−/− mice loaded with the intracellular calcium dyes Fluo-4 and FuraRed to assess the relationship between changes in intracellular and mitochondrial calcium levels. Data was acquired on a BD™ LSR II flow cytometer using FACSDiva™ software and analyzed with Flowjo (Treestar).

TNP-Ficoll Immunization:

To elicit T cell-independent type 2 antibody responses, age- and sex-matched C57BL/6 and Cacnalf−/− mice were injected intraperitoneally with 50 μg of 2,4,6-trinitrophenol (TNP)-aminoethyl carboxymethyl (AECM)-Ficoll (Biosearch Technologies). Sera were collected before immunization and 7 days after injection and analyzed by enzyme-linked immunosorbent assay (ELISA). ELISA plates were coated overnight at 4° C. with TNP-BSA, washed and blocked with 1% (vol/vol) BSA for 1 h at 37° C. Serial dilutions of serum samples were then added and incubated for 1 h at 37° C. After plates were washed, horseradish peroxidase-conjugated anti-mouse IgM or anti-mouse IgG3 (Southern Biotech) was added and further incubated for 1 h at 37° C., followed by reaction with SureBlue Reserve tetramethylbenzidine substrate solution (KPL) and measuring absorbance at 450 nm.

Statistical Analysis.

Statistical significance was calculated with Graphpad Prism software using two-tailed unpaired Student's t test. A value of p<0.05 was considered significant. Data are represented as means±SD.

### Results

### Caᵥ1.4-Deficient Mice Show Normal B Lymphocyte Development in the Bone Marrow.

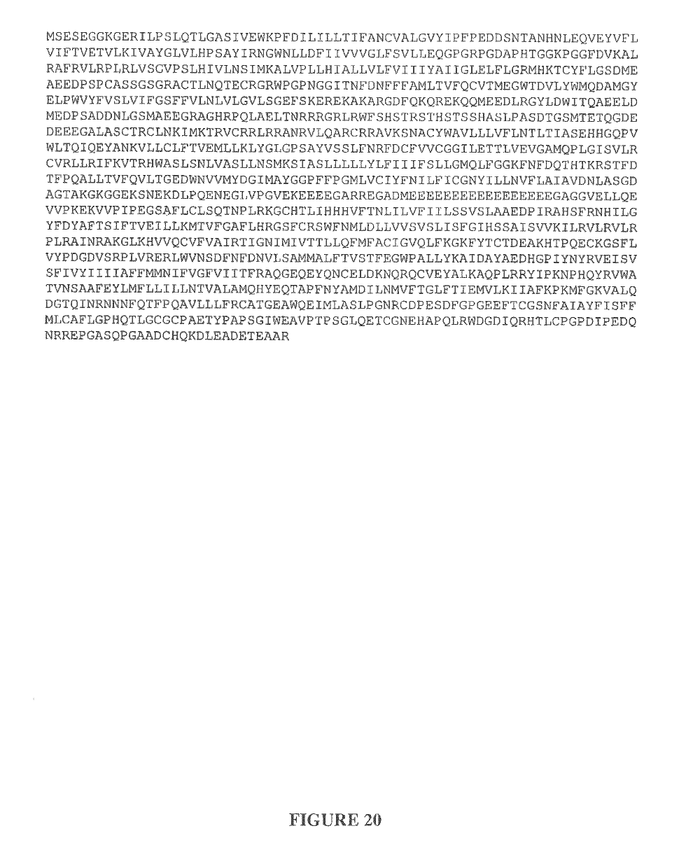

FIG. 22A demonstrates that Caᵥ1.4-deficient mice have unaltered frequency and numbers of B lymphocytes in the bone marrow. The frequencies (percentage of lymphocytes) and total numbers of B lymphocytes in the bone marrow were determined by flow cytometry analysis of bone marrow cells labeled with B220 antibody. FIG. 22B demonstrates that Caᵥ1.4-deficient mice have unaltered progression from pre-pro-B cell stage to the immature stage but have markedly reduced numbers of recirculating mature B lymphocytes in the bone marrow. Total numbers of each B lymphocyte (B220+) subset in the bone marrow were determined by flow cytometry analysis of cells labeled with various antibodies. Populations were defined according to the Hardy gating scheme: pre-pro-B, B220+CD43+BP-1−HSA−; pro-B, B220+CD43+BP-1−HSA+; early pre-B, B220+CD43+BP-1+HSA+; late pre-B, B220+CD43−IgM−IgD−; immature pre-B, B220+CD43−IgM+IgD−; and recirculating mature B cells (mature), B220+CD43−IgM+IgD+. ** p<0.01.

### Caᵥ1.4-Deficient Mice Show Altered Splenic B Lymphocyte Maturation.

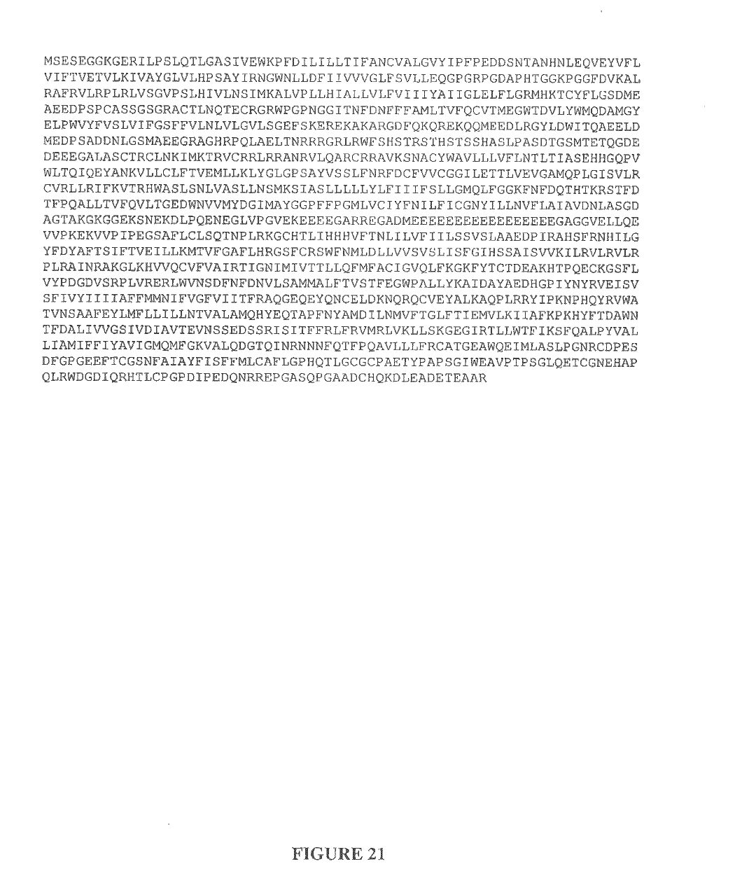

FIG. 23A demonstrates that Caᵥ1.4-deficient mice exhibit reduced frequency and numbers of splenic B cells. The frequencies (percentage of lymphocytes) and total numbers of B lymphocytes in the spleen were determined by flow cytometry analysis of splenocytes labeled with B220 antibody. FIG. 23B demonstrates that Caᵥ1.4-deficient mice exhibit altered percentages of splenic B cell subsets with dramatically reduced frequency and numbers of marginal zone B cells. The frequencies and total numbers of each B lymphocyte (B220+) subset in the spleen were determined by flow cytometry analysis of splenocytes labeled with antibodies to the indicated surface molecules. B cell populations were defined as following: transitional T1, CD93+ CD23− IgMhigh IgD−/low CD21/35−/low; transitional T2, CD93+ CD23+ IgMhigh IgDhigh CD21/35low; transitional T3, CD93+ CD23+ IgMlow IgDhigh CD21/35low; follicular type I (Fo1), CD93− CD23+ IgMlow IgDhigh CD21/35int.; follicular type II (Fo2), CD93−/low CD23+ IgMhigh IgDhigh CD21/35int.; marginal zone precursor (MZP) CD93−/low CD23+ sIgMhigh CD1d+ IgDhigh CD21/35high; and marginal zone (MZ) CD93− CD23− IgMhigh IgDlow CD21/35high. * p<0.05, ** p<0.01 and *** p<0.001

### Caᵥ1.4-Deficiency Results in Altered Peritoneal Cavity B Cell Compartment.

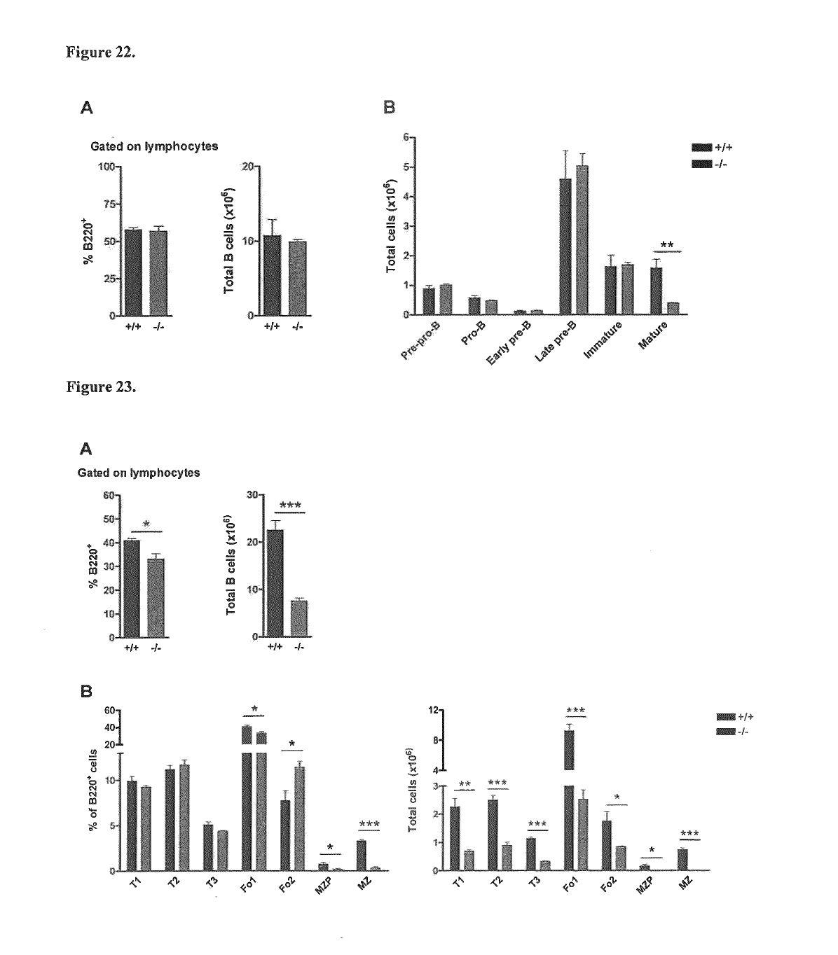

FIG. 24 demonstrates that Caᵥ1.4-deficiency results in altered peritoneal cavity B cell compartment. A. The frequency (percentage of lymphocytes) of B lymphocytes in the peritoneal cavity was determined by flow cytometry analysis of cells labeled with B220 antibody. B. The percentages of each B lymphocyte (B220+) subset in the peritoneal cavity were determined by flow cytometry analysis of cells labeled with B220, CD11b and CD5 antibodies. B cell populations were defined as following: conventional B2 B cells, B220+CD11b−; B1a B cells, B220+CD11b+CD5+; and B1b B cells, B220+CD11b+CD5−. ** p<0.01 and *** p<0.001.

### A Cell-Intrinsic Caᵥ1.4 Function is Required for Normal B Cell Development.

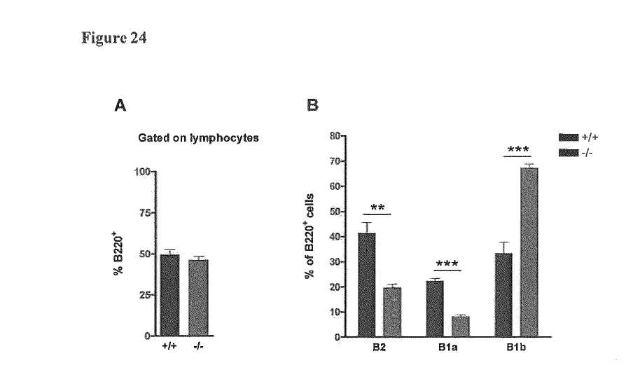

FIG. 25 demonstrates that a cell-intrinsic Caᵥ1.4 function is required for normal B cell development Flow cytometry of B cell development in lethally irradiated congenic CD45.1+ wild-type recipient mice intravenously injected with a 1:1 mixture of wild-type CD45.1+CD45.2+ (competitor) plus wild-type CD45.2+ (donor) bone marrow or wild-type CD45.1+CD45.2+ (competitor) plus Cacnalf−/− CD45.2+ (donor) bone marrow analyzed at 8 weeks after reconstitution. Results are presented as the ratio of CD45.2+ donor lymphocytes to CD45.1+CD45.2+ competitor lymphocytes (+/+ blue squares, wild-type CD45.2+ donor to CD45.1+CD45.2+ competitor cell; −/− red triangles, Cacnalf−/− CD45.2+ donor to CD45.1+CD45.2+ competitor cells) in the bone marrow (A), spleen (B) and peritoneal cavity (C). B cell populations were defined as following: in the bone marrow, total B cells, B220+; pro-B cells, B220+IgM−CD43+; pre-B cells, B220+IgM−CD43−; immature B cells, B220lowIgM+; and recirculating mature B cells, B220highIgM+; in the spleen, transitional T1 B cells, B220+IgM+CD21−CD23−; transitional T2 B cells, B220⁺IgM⁺CD21⁺CD23⁺; follicular B cells, B220⁺IgM^{lo}CD21^{mid}; and marginal zone B cells, B220⁺IgM⁺CD21⁺CD23⁻; and in the peritoneal cavity, conventional B2 B cells, B220+CD11b−; B1a B cells, B220+CD11b+CD5+; and B1b B cells, B220+CD11b+CD5−.

Caᵥ1.4-Deficiency Results in Impaired B Cell Receptor- and Thapsigargin-Induced Ca²⁺ Responses in B Cells.

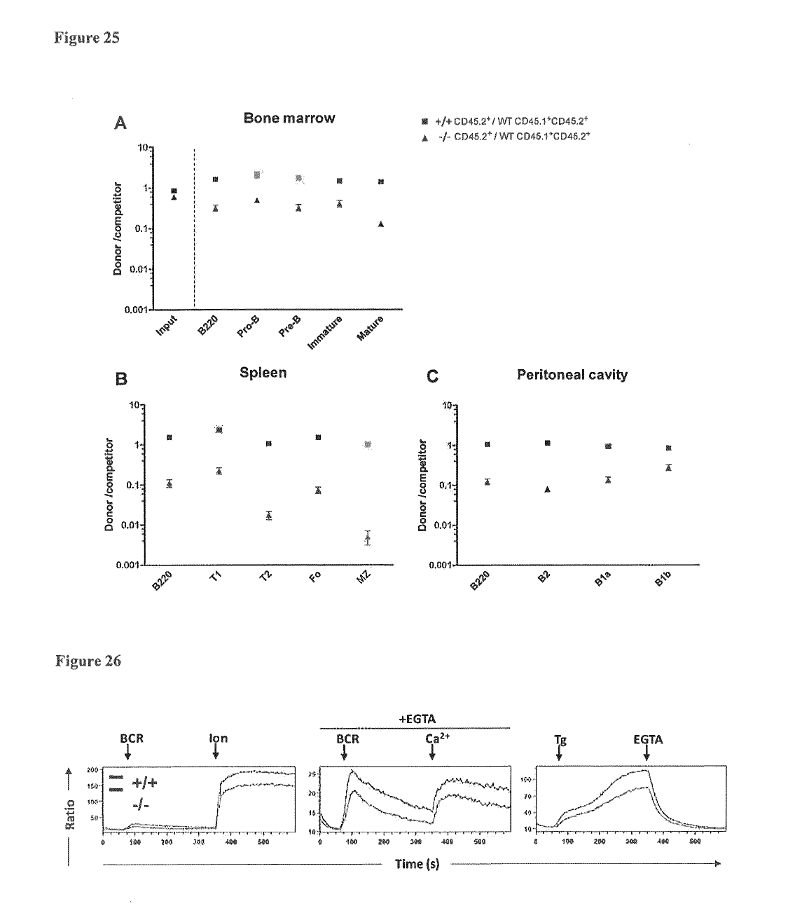

FIG. 26 demonstrates that Caᵥ1.4-deficiency results in impaired B cell receptor- and thapsigargin-induced Ca²⁺ responses in B cells. Wild-type (+/+, blue line) and Cacnalf−/−(−/−, red line) splenocytes were loaded with the intracellular Ca²⁺ dyes Fluo-4 and FuraRed, surface stained with B220 antibody and analyzed by flow cytometry. The intracellular Ca²⁺ levels in splenic B lymphocytes (B220+) were plotted as the ratio of Fluo-4/FuraRed over time. Splenic B lymphocytes were stimulated with anti-IgM (BCR), ionomycin (Ion) or thapsigargin (Tg) at the indicated time points. Extracellular Ca²⁺ was chelated by EGTA addition.

Caᵥ1.4-Deficiency Results in Impaired B Cell Receptor-Induced Mitochondrial Ca²⁺ Responses.

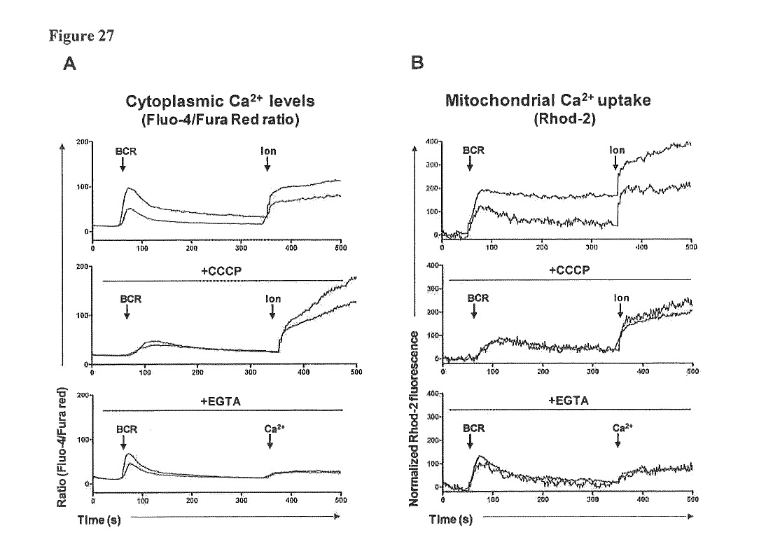

FIG. 27 demonstrates that Caᵥ1.4-deficiency results in impaired B cell receptor-induced mitochondrial Ca²⁺ responses. Wild-type (+/+, blue line) and Cacnalf−/−(−/−, red line) splenocytes were loaded with the intracellular Ca²⁺ dyes Fluo-4 and FuraRed (A) or with the mitochondrial Ca²⁺ dye Rhod-2 (B), surface stained with B220 antibody and analyzed by flow cytometry. The intracellular Ca²⁺ levels in splenic B lymphocytes (B220+) were plotted as the ratio of Fluo-4/FuraRed over time. Cells were stimulated with anti-IgM (BCR) or ionomycin (Ion) at the indicated time points in the presence or absence of carbonyl cyanide 3-chlorophenylhydrazone (CCCP) to disrupt the mitochondrial membrane potential or EGTA to chelate extracellular Ca²⁺.

### Caᵥ1.4-Deficient B Cells Show Defective B Cell Receptor-Mediated Activation.

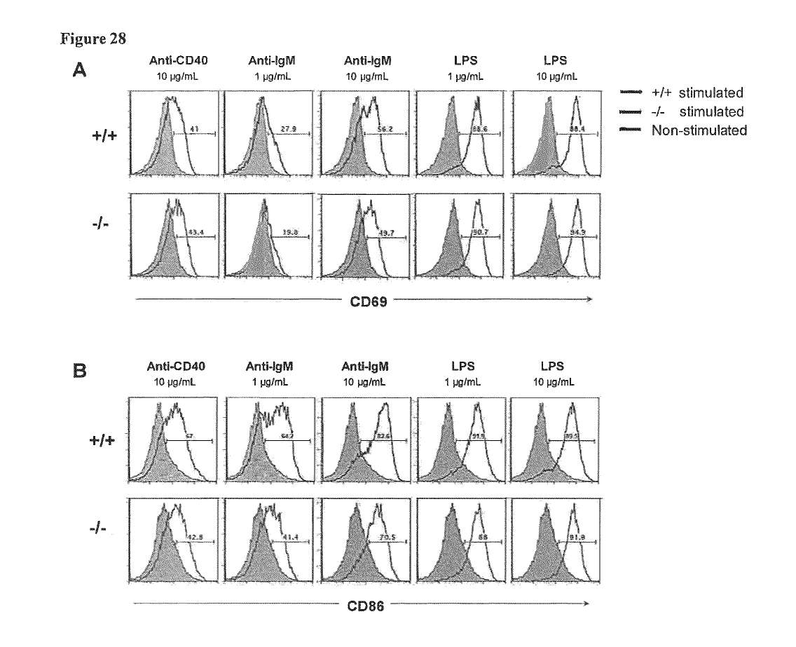

FIG. 28 demonstrates that Caᵥ1.4-deficient B cells show defective B cell receptor-mediated activation. Wild-type (+/+, blue line) and Cacnalf−/−(−/−, red line) splenocytes were left unstimulated (grey) or stimulated with anti-IgM, anti-CD40 or LPS at the indicated concentrations for 24 h, surface stained with B220, CD69 (A) and CD86 (B) antibodies and analyzed by flow cytometry. Numbers above bracketed lines represent the percentage of splenic B cells (B220+) that have upregulated the surface marker.

### Caᵥ1.4-Deficient B Cells Show Reduced B Cell Receptor-Induced Proliferation.

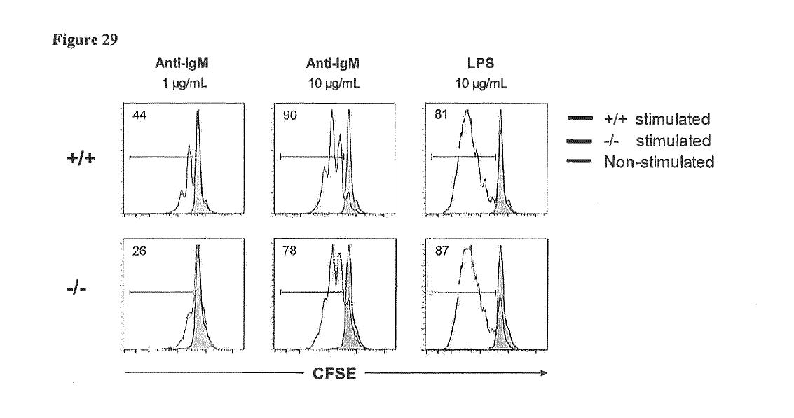

FIG. 29 demonstrates that Caᵥ1.4-deficient B cells show reduced B cell receptor-induced proliferation. Wild-type (+/+, blue line) and Cacnalf−/−(−/−, red line) CFSE-labeled splenocytes were left unstimulated (grey) or stimulated with anti-IgM or LPS at the indicated concentrations for 72 h and then surface stained with B220 antibody and analyzed by flow cytometry. Numbers above bracketed lines represent the percentage of dividing cells.

### Caᵥ1.4-Deficient Splenic B Cells Show Reduced Expression of B Cell Activating Factor (BAFF) Receptor and Lower Survival Rates in Response to BAFF.

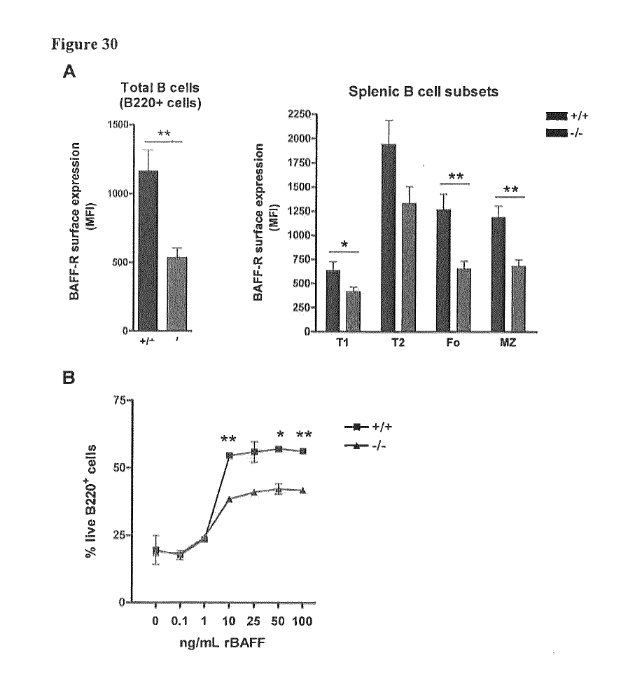

FIG. 30 demonstrates that Caᵥ11.4-deficient splenic B cells show reduced expression of B cell activating factor (BAFF) receptor and lower survival rates in response to BAFF. A. Flow cytometry analysis of surface expression of BAFF receptor in total B220+ splenic B cells and in splenic B cell subsets from wild-type (+/+, black) and Cacnalf−/−(−/−, grey) mice. B cell populations were defined as following: transitional T1 B cells, B220⁺IgM⁺CD21⁻CD23⁻; transitional T2 B cells, B220⁺IgM⁺CD21⁺CD23⁺; follicular B cells, B220⁺IgM^{lo}CD21^{mid}; and marginal zone B cells, B220⁺IgM⁺CD21⁺CD23⁻. B. Purified splenic B cells from wild-type (+/+, blue line) and Cacnalf−/−(−/−, red line) mice were cultured in the presence or absence of the indicated concentrations of recombinant mouse BAFF for 72 h and then stained with propidium iodide. The percentage of live cells (propidium iodide negative cells) was assessed by flow cytometry. * p<0.05 and ** p<0.01.

Caᵥ1.4-Deficient Mice Generate Impaired Antibody Responses after Immunization with TNP-Ficoll, a T Cell-Independent Type-2 Antigen.

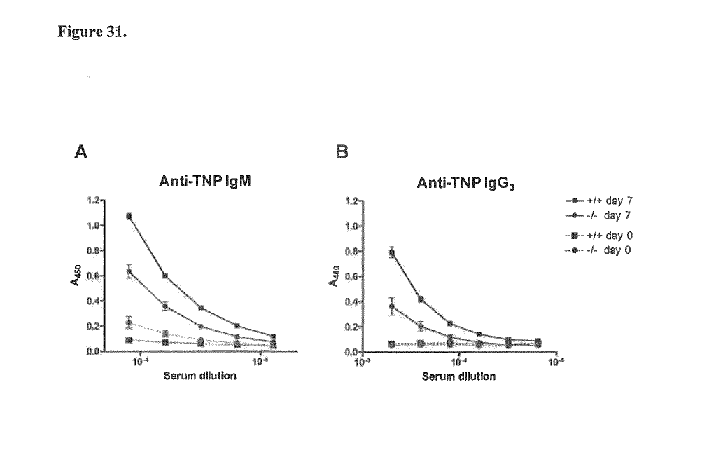

FIG. 31 demonstrates that Caᵥ1.4-deficient mice generate impaired antibody responses after immunization with TNP-Ficoll, a T cell-independent type-2 antigen. Wild-type (n=5) and Cacnalf−/−(n=5) mice were injected intraperitoneally with TNP-Ficoll and the levels of specific antibodies elicited after the immunization were determined by ELISA. TNP-specific anti-IgM (A) and anti-IgG3 (B) antibody responses on day 0 (wild-type, +/+ black line; Cacnalf−/−, −/− grey line) and on day 7 after immunization (wild-type, +/+ blue line; Cacnalf−/−, −/− red line).

### REFERENCES

Alberola-Ila, J., and Hernández-Hoyos, G. (2003). The Ras/MAPK cascade and the control of positive selection. Immunol. Rev. 191, 79-96.
Badou, A., Jha, M. K., Matza, D., Mehal, W. Z., Freichel, M., Flockerzi, V., and Flavell, R. A. (2006). Critical role for the beta regulatory subunits of Cav channels in T lymphocyte function. Proc. Natl. Acad. Sci. USA 103, 15529-15534.
Baumann, L., Gerstner, A., Zong, X., Biel, M., and Wahl-Schott, C. (2004). Functional characterization of the L-type Ca2+ channel Cav1.4alpha1 from mouse retina. Invest. Ophthalmol. Vis. Sci. 45, 708-713.
Burgess, D. L., Jones, J. M., Meisler, M. H., and Noebels, J. L. (1997). Mutation of the Ca2+ channel beta subunit gene Cchb4 is associated with ataxia and seizures in the lethargic (lh) mouse. Cell 88, 385-392.
Catterall, W. A. (2000). Structure and regulation of voltage-gated Ca2+ channels. Annu. Rev. Cell Dev. Biol. 16, 521-555.
David, M. D., Cochrane, C. L., Duncan, S. K., and Schrader, J. W. (2005). Pure lipopolysaccharide or synthetic lipid A induces activation of p21Ras in primary macrophages through a pathway dependent on Src family kinases and PI3K. J. Immunol. 175, 8236-8241.
Feske, S. (2007). Calcium signalling in lymphocyte activation and disease. Nat. Rev. Immunol. 7, 690-702.
Grafton, G., Stokes, L., Toellner, K. M., and Gordon, J. (2003). A non-voltage-gated calcium channel with L-type characteristics activated by B cell receptor ligation. Biochem. Pharmacol. 66, 2001-2009.
Gwack, Y., Srikanth, S., Oh-Hora, M., Hogan, P. G., Lamperti, E. D., Yamashita, M., Gelinas, C., Neems, D. S., Sasaki, Y., Feske, S., et al. (2008). Hair loss and defective T- and B-cell function in mice lacking ORAI1. Mol. Cell. Biol. 28, 5209-5222.
Jha, M. K., Badou, A., Meissner, M., McRory, J. E., Freichel, M., Flockerzi, V., and Flavell, R. A. (2009). Defective survival of naive CD8+ T lymphocytes in the absence of the beta3 regulatory subunit of voltage-gated calcium channels. Nat. Immunol. 10, 1275-1282.
Kieper, W. C., Troy, A., Burghardt, J. T., Ramsey, C., Lee, J. Y., Jiang, H. Q., Dummer, W., Shen, H., Cebra, J. J., and Surh, C. D. (2005). Recent immune status determines the source of antigens that drive homeostatic T cell expansion. J. Immunol. 174, 3158-3163.
Kotturi, M. F., and Jefferies, W. A. (2005). Molecular characterization of L-type calcium channel splice variants expressed in human T lymphocytes. Mol. Immunol. 42, 1461-1474.
Kotturi, M. F., Carlow, D. A., Lee, J. C., Ziltener, H. J., and Jefferies, W. A. (2003). Identification and functional characterization of voltage-dependent calcium channels in T lymphocytes. J. Biol. Chem. 278, 46949-46960.
Kotturi, M. F., Hunt, S. V., and Jefferies, W. A. (2006). Roles of CRAC and Cav-like channels in T cells: More than one gatekeeper? Trends Pharmacol. Sci. 27, 360-367.
Lipscombe, D., Helton, T. D., and Xu, W. (2004). L-type calcium channels: The low down. J. Neurophysiol. 92, 2633-2641.
Liu, K. Q., Bunnell, S. C., Gurniak, C. B., and Berg, L. J. (1998). T cell receptor-initiated calcium release is uncoupled from capacitative calcium entry in Itk-deficient T cells. J. Exp. Med. 187, 1721-1727.
Mansergh, F., Orton, N. C., Vessey, J. P., Lalonde, M. R., Stell, W. K., Tremblay, F., Barnes, S., Rancourt, D. E., and Bech-Hansen, N. T. (2005). Mutation of the calcium channel gene Cacnalf disrupts calcium signaling, synaptic transmission and cellular organization in mouse retina. Hum. Mol. Genet. 14, 3035-3046.
McRory, J. E., Hamid, J., Doering, C. J., Garcia, E., Parker, R., Hamming, K., Chen, L., Hildebrand, M., Beedle, A. M., Feldcamp, L., et al. (2004). The CACNAIF gene encodes an L-type calcium channel with unique biophysical properties and tissue distribution. J. Neurosci. 24, 1707-1718.
Morgan, A. J., and Jacob, R. (1994). Ionomycin enhances Ca2+ influx by stimulating store-regulated cation entry and not by a direct action at the plasma membrane. Biochem. J. 300, 665-672.
Oh-hora, M. (2009). Calcium signaling in the development and function of T-lineage cells. Immunol. Rev. 231, 210-224.
Park, C. Y., Shcheglovitov, A., and Dolmetsch, R. (2010). The CRAC channel activator STIM1 binds and inhibits L-type voltage-gated calcium channels. Science 330, 101-105.
Priatel, J. J., Chui, D., Hiraoka, N., Simmons, C. J., Richardson, K. B., Page, D. M., Fukuda, M., Varki, N. M., and Marth, J. D. (2000). The ST3Gal-I sialyltransferase controls CD8+ T lymphocyte homeostasis by modulating O-glycan biosynthesis. Immunity 12, 273-283.
Priatel, J. J., Teh, S. J., Dower, N. A., Stone, J. C., and Teh, H. S. (2002). RasGRP1 transduces low-grade TCR signals which are critical for T cell development, homeostasis, and differentiation. Immunity 17, 617-627.
Priatel, J. J., Chen, X., Dhanji, S., Abraham, N., and Teh, H. S. (2006). RasGRP1 transmits prodifferentiation TCR signaling that is crucial for CD4 T cell development. J. Immunol. 177, 1470-1480.
Priatel, J. J., Chen, X., Zenewicz, L. A., Shen, H., Harder, K. W., Horwitz, M. S., and Teh, H. S. (2007). Chronic immunodeficiency in mice lacking RasGRP1 results in CD4 T cell immune activation and exhaustion. J. Immunol. 179, 2143-2152.
Revy, P., Sospedra, M., Barbour, B., and Trautmann, A. (2001). Functional antigen-independent synapses formed between T cells and dendritic cells. Nat. Immunol. 2, 925-931.
Stokes, L., Gordon, J., and Grafton, G. (2004). Non-voltage-gated L-type Ca2+ channels in human T cells: pharmacology and molecular characterization of the major alpha pore-forming and auxiliary beta-subunits. J. Biol. Chem. 279, 19566-19573.
Strom, T. M., Nyakatura, G., Apfelstedt-Sylla, E., Hellebrand, H., Lorenz, B., Weber, B. H., Wutz, K., Gutwillinger, N., Rither, K., Drescher, B., et al. (1998). An L-type calcium-channel gene mutated in incomplete X-linked congenital stationary night blindness. Nat. Genet. 19, 260-263.
Surh, C. D., and Sprent, J. (2005). Regulation of mature T cell homeostasis. Semin. Immunol. 17, 183-191.
Surh, C. D., and Sprent, J. (2008). Homeostasis of naive and memory T cells. Immunity 29, 848-862.
Suzuki, Y., Yoshimaru, T., Inoue, T., Nunomura, S., and Ra, C. (2008). The high-affinity immunoglobulin E receptor (FcepsilonRI) regulates mitochondrial calcium uptake and a dihydropyridine receptor-mediated calcium influx in mast cells: Role of the FcepsilonRIbeta chain immunoreceptor tyrosine-based activation motif. Biochem. Pharmacol. 75, 1492-1503.
Teixeiro, E., and Daniels, M. A. (2010). ERK and cell death: ERK location and T cell selection. FEBS J. 277, 30-38.
Thastrup, O., Cullen, P. J., Drøbak, B. K., Hanley, M. R., and Dawson, A. P. (1990). Thapsigargin, a tumor promoter, discharges intracellular Ca2+ stores by specific inhibition of the endoplasmic reticulum Ca2(+)-ATPase. Proc. Natl. Acad. Sci. USA 87, 2466-2470.
Vig, M., and Kinet, J. P. (2009). Calcium signaling in immune cells. Nat Immunol. 10, 21-27.
Vig, M., DeHaven, W. I., Bird, G. S., Billingsley, J. M., Wang, H., Rao, P. E., Hutchings, A. B., Jouvin, M. H., Putney, J. W., and Kinet, J. P. (2008). Defective mast cell effector functions in mice lacking the CRACM1 pore subunit of store-operated calcium release-activated calcium channels. Nat. Immunol. 9, 89-96.
Wang, Y., Deng, X., Mancarella, S., Hendron, E., Eguchi, S., Soboloff, J., Tang, X. D., and Gill, D. L. (2010). The calcium store sensor, STIM1, reciprocally controls Orai and CaV1.2 channels. Science 330, 105-109.
Woodard, G. E., Salido, G. M., and Rosado, J. A. (2008). Enhanced exocytotic-like insertion of Oral I into the plasma membrane upon intracellular Ca2+ store depletion. Am. J. Physiol. Cell Physiol. 294, C1323-C1331.

The disclosure of all patents, publications, including published patent applications, and database entries referenced in this specification are expressly incorporated by reference in their entirety to the same extent as if each such individual patent, publication, and database entry were expressly and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method for modulating the function of a cell expressing a Cay1 splice variant comprising contacting the cell with an agent that specifically binds to an ectodomain of the Cay1 splice variant, wherein binding of the agent to the Cay1 splice variant modulates the activity of the Cay1 splice variant and wherein the cell is a haematopoietic cell.

2. The method according to claim 1, wherein the binding of the agent to the Cay1 splice variant inhibits the activity of the Cay1 splice variant.

3. The method according to claim 1, wherein the binding of the agent to the Cay1 splice variant activates the activity of the Cay1 splice variant.

4. The method according to claim 1, wherein the Cay1 splice variant is a Cay1.4 splice variant.

5. The method according to claim 1, wherein the cell is a haematopoietic cell of the lymphoid lineage.

6. The method according to claim 5, wherein the cell is a T cell.

7. The method according to claim 6, wherein the function of the cell comprises T cell maturation.

8. The method according to claim 6, wherein the function of the cell comprises antigen binding.

9. The method according to claim 5, wherein the cell is a B cell.

10. The method according to claim 9, wherein the function of the cell comprises B cell maturation.

11. The method according to claim 9, wherein the function of the cell comprises BCR-induced activation.

12. The method according to claim 1, wherein the agent is an antibody or an aptamer.

13. A method of modulating an immune response in a subject comprising administering to the subject an effective amount of a Cay1 modulator, wherein the Cay1 modulator binds to an ectodomain of a Cay1 splice variant expressed in a haematopoietic cell.

14. The method according to claim 13, wherein the haematopoietic cell is of the lymphoid lineage.

15. The method according to claim 14, wherein the haematopoietic cell is a T cell or B cell.

16. The method according to claim 13, wherein the agent is an antibody or an aptamer.

17. A method of screening for therapeutic agents comprising the steps of:
contacting a haematopoietic cell expressing a Cay1 splice variant with a test agent, and
determining whether the test agent modulates activity of the Cay1 splice variant, wherein a test agent that modulates activity of the Cay1 splice variant is identified as a therapeutic agent.

18. The method according to claim 17, wherein the Cay1 splice variant is a Cay1.4 splice variant.

19. The method according to claim 18, wherein the haematopoietic cell is of the lymphoid lineage.

20. The method according to claim 17, wherein the test agent is an agent capable of binding to an ectodomain of the Cay1 splice variant.

21. The method according to claim 17, wherein the agent is an antibody or an aptamer.

22-28. (canceled)

29. A method of suppressing an immune response in a subject comprising administering to the subject an effective amount of a Caᵥ1.4 inhibitor, wherein the Caᵥ1.4 inhibitor binds to an ectodomain of a Cay1.4 splice variant expressed in T cells and/or B cells.

30. The method according to claim 29, wherein the agent is an antibody or an aptamer.

31. A method of screening for an immunosuppressant comprising the steps of:
contacting T cells and/or B cells expressing a Cay1.4 splice variant with a test agent, and
determining whether the test agent modulates activity of the Cay1.4 splice variant, wherein a test agent that inhibits activity of the Cay1.4 splice variant is identified as an immunosuppressant.

32. The method according to claim 31, wherein the test agent is an agent capable of binding to an ectodomain of the Cay1.4 splice variant.

33. The method according to claim 31, wherein the agent is an antibody or an aptamer.

34. A method for modulating the function of a cell expressing a voltage-gated calcium channel comprising contacting the cell with an agent that specifically binds to the voltage-gated calcium channel, wherein binding of the agent to the voltage-gated calcium channel modulates the activity of the channel and wherein the cell is a haematopoietic cell.

35. A method of modulating an immune response in a subject comprising administering to the subject an effective amount of a voltage-gated calcium channel modulator, wherein the modulator binds to a voltage-gated calcium channel expressed in a haematopoietic cell.


SEQUENCE LISTING (17 sequences)

SEQ ID NO: 1 - protein, 1977 aa, Homo sapiens
MSESEGGKDTTPEPSPANGAGPGPEWGLCPGPPAVEGESSGASGLGTPKRRNQHSKHKTVAVASAQRSPRALFCLTLANPLRRSCISIVEWKPFDILILLTIFANCVALGVYIPFPEDDSNTANHNLEQVEYVFLVIFTVETVLKIVAYGLVLHPSAYIRNGWNLLDFIIVVVGLFSVLLEQGPGRPGDAPHTGGKPGGFDVKALRAFRVLRPLRLVSGVPSLHIVLNSIMKALVPLLHIALLVLFVIIIYAIIGLELFLGRMHKTCYFLGSDMEAEEDPSPCASSGSGRACTLNQTECRGRWPGPNGGITNFDNFFFAMLTVFQCVTMEGWTDVLYWMQDAMGYELPWVYFVSLVIFGSFFVLNLVLGVLSGEFSKEREKAKARGDFQKQREKQQMEEDLRGYLDWITQAEELDMEDPSADDNLGSMAEEGRAGHRPQLAELTNRRRGRLRWFSHSTRSTHSTSSHASLPASDTGSMTETQGDEDEEEGALASCTRCLNKIMKTRVCRRLRRANRVLRARCRRAVKSNACYWAVLLLVFLNTLTIASEHHGQPVWLTQIQEYANKVLLCLFTVEMLLKLYGLGPSAYVSSFFNRFDCFVVCGGILETTLVEVGAMQPLGISVLRCVRLLRIFKVTRHWASLSNLVASLLNSMKSIASLLLLLFLFIIIFSLLGMQLFGGKFNFDQTHTKRSTFDTFPQALLTVFQILTGEDWNVVMYDGIMAYGGPFFPGMLVCIYFIILFICGNYILLNVFLAIAVDNLASGDAGTAKDKGGEKSNEKDLPQENEGLVPGVEKEEEEGARREGADMEEEEEEEEEEEEEEEEEGAGGVELLQEVVPKEKVVPIPEGSAFFCLSQTNPLRKGCHTLIHHHVFTNLILVFIILSSVSLAAEDPIRAHSFRNHILGYFDYAFTSIFTVEILLKMTVFGAFLHRGSFCRSWFNMLDLLVVSVSLISFGIHSSAISVVKILRVLRVLRPLRAINRAKGLKHVVQCVFVAIRTIGNIMIVTTLLQFMFACIGVQLFKGKFYTCTDEAKHTPQECKGSFLVYPDGDVSRPLVRERLWVNSDFNFDNVLSAMMALFTVSTFEGWPALLYKAIDAYAEDHGPIYNYRVEISVFFIVYIIIIAFFMMNIFVGFVIITFRAQGEQEYQNCELDKNQRQCVEYALKAQPLRRYIPKNPHQYRVWATVNSAAFEYLMFLLILLNTVALAMQHYEQTAPFNYAMDILNMVFTGLFTIEMVLKIIAFKPKHYFTDAWNTFDALIVVGSIVDIAVTEVNNGGHLGESSEDSSRISITFFRLFRVMRLVKLLSKGEGIRTLLWTFIKSFQALPYVALLIAMIFFIYAVIGMQMFGKVALQDGTQINRNNNFQTFPQAVLLLFRCATGEAWQEIMLASLPGNRCDPESDFGPGEEFTCGSNFAIAYFISFFMLCAFLIINLFVAVIMDNFDYLTRDWSILGPHHLDEFKRIWSEYDPGAKGRIKHLDVVALLRRIQPPLGFGKLCPHRVACKRLVAMNMPLNSDGTVTFNATLFALVRTSLKIKTEGNLEQANQELRIVIKKIWKRMKQKLLDEVIPPPDEEEVTVGKFYATFLIQDYFRKFRRRKEKGLLGNDAAPSTSSALQAGLRSLQDLGPEMRQALTCDTEEEEEEGQEGVEEEDEKDLETNKATMVSQPSARRGSGISVSLPVGDRLPDSLSFGPSDDDRGTPTSSQPSVPQAGSNTHRRGSGALIFTIPEEGNSQPKGTKGQNKQDEDEEVPDRLSYLDEQAGTPPCSVLLPPHRAQRYMDGHLVPRRRLLPPTPAGRKPSFTIQCLQRQGSCEDLPIPGTYHRGRNSGPNRAQGSWATPPQRGRLLYAPLLLVEEGAAGEGYLGRSSGPLRTFTCLHVPGTHSDPSHGKRGSADSLVEAVLISEGLGLFARDPRFVALAKQEIADACRLTLDEMDNAASDLLAQGTSSLYSDEESILSRFDEEDLGDEMACVHAL

SEQ ID NO: 2 - DNA, 5385 bp, Homo sapiens
atgtcggaatctgaaggcgggaaaggtgagagaatccttccatccctgcagacccttggagcaagcatcgtggagtggaagcccttcgacatcctcatcctgctgaccatctttgccaactgcgtggccctgggagtttacatccccttccctgaggacgactccaacactgccaaccacaacctggagcaggtggagtacgtattcctggtgattttcactgtggagacggtgctcaagatcgtggcctacgggctggtgctccaccccagcgcctacatccgcaatggctggaacctactcgacttcatcatcgtcgtggtcgggctgttcagcgttctgctggagcagggccccggacggccaggcgacgccccgcacaccgggggaaagccaggaggcttcgatgtgaaggcattgagggcgtttcgggtgctgcggccactgaggctggtgtctggggtcccgagcctgcacatagtgctcaattccatcatgaaggctctggtgccgctgctgcacattgcactgctcgtgctcttcgtcatcatcatttatgccatcattgggctcgagctgttccttggacgaatgcacaagacgtgctacttcctgggatccgacatggaagcggaggaggacccatcgccctgtgcgtcttcgggatcagggcgtgcgtgcacgctgaaccagactgagtgccgcgggcgctggccagggcccaatggaggcatcaccaactttgacaacttcttcttcgccatgctgacagtcttccagtgtgtcaccatggaaggctggaccgatgtgctctactggatgcaagatgccatggggtatgaactgccctgggtgtactttgtgagccttgtcatctttgggtccttcttcgtcctcaaccttgtgcttggcgtcctgagtggggagttctccaaggagagagagaaagcgaaagctcgcggggacttccagaagcagcgggagaagcagcagatggaggaagacctgcggggctacctggactggatcactcaagccgaagagctggacatggaggacccctccgccgatgacaaccttggttctatggctgaagagggccgggcgggccatcggccacagctggccgagctgaccaataggaggcgtggacgtctgcgctggttcagtcattctactcgctccacacactccaccagcagccatgccagcctcccagccagtgacaccggttccatgacagagacccaaggcgatgaggatgaggaggagggggctctggccagctgtacacgctgcctgaacaagatcatgaaaaccagagtctgccgccgcctccgccgagccaaccgggtccttcaggcacgctgccgtcgggcagtgaagtccaatgcctgctactgggctgtgctgttgctcgtcttcctcaacacgttgaccatcgcctctgagcaccacgggcagcctgtgtggctcacccagatccaggagtatgccaacaaagtgttgctctgtctgttcacggtggagatgcttctcaaattgtacggtctgggcccctctgcctatgtgtcttccctcttcaaccgctttgactgctttgtggtctgtgggggcatcctagagaccaccttggtggaggtgggtgccatgcagcccttgggcatctcagtgctccgatgtgtgcgcctcctcaggatctttaaggtcaccagacactgggcttctctgagcaatctggtggcatccctgctcaattcaatgaaatccatcgcatccttgctgcttctcctctacctcttcatcattatcttctccctgcttggcatgcagctgtttgggggcaagttcaactttgaccagacccacaccaagcgaagcacctttgacacgttcccccaggccctcctcactgtctttcaggtcctgacaggtgaggactggaacgtggtcatgtatgatggtatcatggcatatggtggccccttcttcccaggaatgttggtgtgcatctatttcaacattctcttcatctgtggcaactacatcctgttgaacgtgtttcttgccattgctgtggacaacctggccagtggagatgcaggcactgccaagggcaagggcggggagaagagcaatgagaaggatctcccacaggagaatgaaggcctggtgcctggtgtggagaaagaggaagaggagggtgcaaggagggaaggagcagacatggaggaggaggaggaggaggaagaagaggaagaagaggaagaagaggaagagggtgcagggggtgtggaactcctgcaggaagttgtacccaaggagaaggtggtacccatccctgagggcagcgccttcctctgcctcagccaaaccaacccgctgaggaagggctgccacaccctcatccaccatcatgtcttcaccaatcttatcctggtgttcatcatcctcagcagtgtgtccctggccgctgaagaccccatccgagcccactccttccgcaaccacattctgggttacttcgattatgccttcacctccattttcactgtggagattctactaaagatgacagtgtttggggccttcctgcaccgcggctccttctgccgtagctggtttaatatgttggatctgctggtggtcagtgtgtccctcatctcctttggcatccactccagcgccatctcggtggtgaagattctgcgagtactccgagtactgcggcccctccgagccatcaacagggccaagggactcaagcacgtggtgcagtgtgtatttgtggccatccggaccatcggaaacatcatgattgtcaccacacttctgcaatttatgttcgcctgcatcggggtgcagctcttcaaggggaaattctacacctgcacggacgaggccaaacacacccctcaagaatgcaagggctccttcctggtatacccagatggagacgtgtcacggcccctggtccgggagcggctctgggtcaacagtgatttcaactttgacaatgtcctttcagccatgatggccctgttcactgtctccacctttgaaggctggccagcactgctatacaaggccatcgatgcatatgcagaggaccacggccccatctataattaccgtgtggagatctcagtgtccttcattgtctacatcatcatcattgcgttcttcatgatgaacatcttcgtgggcttcgtcatcatcactttccgtgcccagggcgagcaggagtaccaaaactgtgagctggacaagaaccagcgtcaatgtgtggaatatgccctcaaggcccagccactccgccgttacatccccaagaacccgcatcagtatcgtgtgtgggccactgtgaactctgctgcctttgagtacctgatgttcctgctcatcctgctcaacacagttgccctagccatgcagcactatgagcagactgctcccttcaactatgccatggacatcctcaacatggtcttcactggcctcttcactattgagatggtactcaaaatcatcgccttcaagcccaagatgttcggcaaggtggctcttcaggatggcacacagataaaccgaaacaacaacttccagacctttccacaggctgtgctgcttctgttcaggtgtgccactggtgaggcatggcaggagataatgcttgccagccttcccggaaatcgatgtgatcctgagtctgacttcggccctggtgaagagtttacctgtggtagcaattttgccatcgcctatttcatcagcttcttcatgctctgtgccttcctggggccgcatcaaacacttggatgtggttgccctgctgagacgtatccagccccctctgggatttgggaagctgtgcccacaccgagtggcctgcaagagacttgtggcaatgaacatgcccctcaactcagatgggacggtgacattcaacgccacactctttgccctggtccggacatccctgaagatcaaaacagaagggaacctggagcaagccaaccaggagctgcggattgtcatcaaaaagatctggaagcggatgaaacagaagctgctagatgaggtcatccccccaccagacgaggaggaggtcaccgtgggcaaattctacgccacatttctgatccaggactatttccgcaaattccggcggaggaaagaaaaagggctactaggcaacgacgccgcccctagcacctcttccgcccttcaggctggtctgcggagcctgcaggacttgggtcgtgagatgcggcaggcccccacctgtgacacagaggaggaggaagaagaggggcaggagggagtggaggaggaagatgaaaaggacttggaaactaacaaagccacgatggtctcccagccctcagctcgccggggctccgggatttctgtgtctctgcctgtcggggacagacttccagattcactctcctttgggcccagtgacgatgacagggggactcccacctccagtcagcccagtgtgccccaggctggatccaacacccacaggagaggctctggggctctcattttcaccatcccagaagaaggaaattctcagcccaagggaaccaaagggcaaaacaagcaagatgaggatgaggaagtccctgatcggctttcctacctagatgagcaggcagggactcccccgtgctcagtccttttgccacctcacagagctcagagatacatggatgggcacctggtaccacgccgccgtctgctgccccccacacctgcaggtcggaagccctccttcaccatccagtgtctgcagcgccagggcagttgtgaggatttacccatcccaggcacctatcatcgtgggcgaaattcagggcccaatagggctcagggttcctgggcaacaccacctcagcggggtcggctcctgtatgccccgctgttgttggtggaagagggcgcggcgggggaggggtacctcggcagatccagtggcccactgcgcaccttcacctgtctgcacgtgcctggaacccactcggaccccagccatgggaagaggggcagtgccggcagcttggtggaggctgtgcttatctcagagggtctgggcctctttgctcgagacccacgtttcgtggccctggccaagcaggagattgcagatgcgtgtcgcctgacgctggatgagatggacaatgctgccagtgacctgctggcacagggaaccagctctctctatagcgacgaggagtccatcctctcccgcttcgatgaggaggacttgggagacgagatggcctgcgtccacgccctctgaattcccacccctccccaactgctcaataaacctcccgccctcccctccccagcaggaggcaggcatggaccaca

SEQ ID NO: 3 - DNA, 5664 bp, Homo sapiens
atgtcggaatctgaaggcgggaaaggtgagagaatccttccatccctgcagacccttggagcaagcatcgtggagtggaagcccttcgacatcctcatcctgctgaccatctttgccaactgcgtggccctgggagtttacatccccttccctgaggacgactccaacactgccaaccacaacctggagcaggtggagtacgtattcctggtgattttcactgtggagacggtgctcaagatcgtggcctacgggctggtgctccaccccagcgcctacatccgcaatggctggaacctactcgacttcatcatcgtcgtggtcgggctgttcagcgttctgctggagcagggccccggacggccaggcgacgccccgcacaccgggggaaagccaggaggcttcgatgtgaaggcattgagggcgtttcgggtgctgcggccactgaggctggtgtctggggtcccgagcctgcacatagtgctcaattccatcatgaaggctctggtgccgctgctgcacattgcactgctcgtgctcttcgtcatcatcatttatgccatcattgggctcgagctgttccttggacgaatgcacaagacgtgctacttcctgggatccgacatggaagcggaggaggacccatcgccctgtgcgtcttcgggatcagggcgtgcgtgcacgctgaaccagactgagtgccgcgggcgctggccagggcccaatggaggcatcaccaactttgacaacttcttcttcgccatgctgacagtcttccagtgtgtcaccatggaaggctggaccgatgtgctctactggatgcaagatgccatggggtatgaactgccctgggtgtactttgtgagccttgtcatctttgggtccttcttcgtcctcaaccttgtgcttggcgtcctgagtggggagttctccaaggagagagagaaagcgaaagctcgcggggacttccagaagcagcgggagaagcagcagatggaggaagacctgcggggctacctggactggatcactcaagccgaagagctggacatggaggacccctccgccgatgacaaccttggttctatggctgaagagggccgggcgggccatcggccacagctggccgagctgaccaataggaggcgtggacgtctgcgctggttcagtcattctactcgctccacacactccaccagcagccatgccagcctcccagccagtgacaccggttccatgacagagacccaaggcgatgaggatgaggaggagggggctctggccagctgtacacgctgcctgaacaagatcatgaaaaccagagtctgccgccgcctccgccgagccaaccgggtccttcaggcacgctgccgtcgggcagtgaagtccaatgcctgctactgggctgtgctgttgctcgtcttcctcaacacgttgaccatcgcctctgagcaccacgggcagcctgtgtggctcacccagatccaggagtatgccaacaaagtgttgctctgtctgttcacggtggagatgcttctcaaattgtacggtctgggcccctctgcctatgtgtcttccctcttcaaccgctttgactgctttgtggtctgtgggggcatcctagagaccaccttggtggaggtgggtgccatgcagcccttgggcatctcagtgctccgatgtgtgcgcctcctcaggatctttaaggtcaccagacactgggcttctctgagcaatctggtggcatccctgctcaattcaatgaaatccatcgcatccttgctgcttctcctctacctcttcatcattatcttctccctgcttggcatgcagctgtttgggggcaagttcaactttgaccagacccacaccaagcgaagcacctttgacacgttcccccaggccctcctcactgtctttcaggtcctgacaggtgaggactggaacgtggtcatgtatgatggtatcatggcatatggtggccccttcttcccaggaatgttggtgtgcatctatttcaacattctcttcatctgtggcaactacatcctgttgaacgtgtttcttgccattgctgtggacaacctggccagtggagatgcaggcactgccaagggcaagggcggggagaagagcaatgagaaggatctcccacaggagaatgaaggcctggtgcctggtgtggagaaagaggaagaggagggtgcaaggagggaaggagcagacatggaggaggaggaggaggaggaagaagaggaagaagaggaagaagaggaagagggtgcagggggtgtggaactcctgcaggaagttgtacccaaggagaaggtggtacccatccctgagggcagcgccttcctctgcctcagccaaaccaacccgctgaggaagggctgccacaccctcatccaccatcatgtcttcaccaatcttatcctggtgttcatcatcctcagcagtgtgtccctggccgctgaagaccccatccgagcccactccttccgcaaccacattctgggttacttcgattatgccttcacctccattttcactgtggagattctactaaagatgacagtgtttggggccttcctgcaccgcggctccttctgccgtagctggtttaatatgttggatctgctggtggtcagtgtgtccctcatctcctttggcatccactccagcgccatctcggtggtgaagattctgcgagtactccgagtactgcggcccctccgagccatcaacagggccaagggactcaagcacgtggtgcagtgtgtatttgtggccatccggaccatcggaaacatcatgattgtcaccacacttctgcaatttatgttcgcctgcatcggggtgcagctcttcaaggggaaattctacacctgcacggacgaggccaaacacacccctcaagaatgcaagggctccttcctggtatacccagatggagacgtgtcacggcccctggtccgggagcggctctgggtcaacagtgatttcaactttgacaatgtcctttcagccatgatggccctgttcactgtctccacctttgaaggctggccagcactgctatacaaggccatcgatgcatatgcagaggaccacggccccatctataattaccgtgtggagatctcagtgtccttcattgtctacatcatcatcattgcgttcttcatgatgaacatcttcgtgggcttcgtcatcatcactttccgtgcccagggcgagcaggagtaccaaaactgtgagctggacaagaaccagcgtcaatgtgtggaatatgccctcaaggcccagccactccgccgttacatccccaagaacccgcatcagtatcgtgtgtgggccactgtgaactctgctgcctttgagtacctgatgttcctgctcatcctgctcaacacagttgccctagccatgcagcactatgagcagactgctcccttcaactatgccatggacatcctcaacatggtcttcactggcctcttcactattgagatggtactcaaaatcatcgccttcaagcccaagcattacttcactgatgcctggaacacgtttgacgctcttattgtggtgggcagcatagtggatattgccgtcactgaagtcaatagctctgaggacagctcccgcatttccattaccttctttcgcctcttccgagttatgcggctggtcaagcttctcagtaagggtgaagggatccgcacattgctctggacattcatcaagtccttccaggccttgccctatgtggctcttctcatcgcaatgatattcttcatctatgccgtcattggcatgcagatgttcggcaaggtggctcttcaggatggcacacagataaaccgaaacaacaacttccagacctttccacaggctgtgctgcttctgttcaggtgtgccactggtgaggcatggcaggagataatgcttgccagccttcccggaaatcgatgtgatcctgagtctgacttcggccctggtgaagagtttacctgtggtagcaattttgccatcgcctatttcatcagcttcttcatgctctgtgccttcctggggccgcatcaaacacttggatgtggttgccctgctgagacgtatccagccccctctgggatttgggaagctgtgcccacaccgagtggcctgcaagagacttgtggcaatgaacatgcccctcaactcagatgggacggtgacattcaacgccacactctttgccctggtccggacatccctgaagatcaaaacagaagggaacctggagcaagccaaccaggagctgcggattgtcatcaaaaagatctggaagcggatgaaacagaagctgctagatgaggtcatccccccaccagacgaggaggaggtcaccgtgggcaaattctacgccacatttctgatccaggactatttccgcaaattccggcggaggaaagaaaaagggctactaggcaacgacgccgcccctagcacctcttccgcccttcaggctggtctgcggagcctgcaggacttgggtcgtgagatgcggcaggcccccacctgtgacacagaggaggaggaagaagaggggcaggagggagtggaggaggaagatgaaaaggacttggaaactaacaaagccacgatggtctcccagccctcagctcgccggggctccgggatttctgtgtctctgcctgtcggggacagacttccagattcactctcctttgggcccagtgacgatgacagggggactcccacctccagtcagcccagtgtgccccaggctggatccaacacccacaggagaggctctggggctctcattttcaccatcccagaagaaggaaattctcagcccaagggaaccaaagggcaaaacaagcaagatgaggatgaggaagtccctgatcggctttcctacctagatgagcaggcagggactcccccgtgctcagtccttttgccacctcacagagctcagagatacatggatgggcacctggtaccacgccgccgtctgctgccccccacacctgcaggtcggaagccctccttcaccatccagtgtctgcagcgccagggcagttgtgaggatttacccatcccaggcacctatcatcgtgggcgaaattcagggcccaatagggctcagggttcctgggcaacaccacctcagcggggtcggctcctgtatgccccgctgttgttggtggaagagggcgcggcgggggaggggtacctcggcagatccagtggcccactgcgcaccttcacctgtctgcacgtgcctggaacccactcggaccccagccatgggaagaggggcagtgccggcagcttggtggaggctgtgcttatctcagagggtctgggcctctttgctcgagacccacgtttcgtggccctggccaagcaggagattgcagatgcgtgtcgcctgacgctggatgagatggacaatgctgccagtgacctgctggcacagggaaccagctctctctatagcgacgaggagtccatcctctcccgcttcgatgaggaggacttgggagacgagatggcctgcgtccacgccctctgaattcccacccctccccaactgctcaataaacctcccgccctcccctccccagcaggaggcaggcatggaccaca

SEQ ID NO: 4 - protein, 50 aa, Homo sapiens [sequence omitted]

SEQ ID NO: 5 - protein, 50 aa, Homo sapiens
PLGFGKFCPHRVACTAAGGHEHAPEQRRHSHLQCHTLCPGPHGTQDQDGR

SEQ ID NO: 6 - protein, 50 aa, Homo sapiens [sequence omitted]

SEQ ID NO: 7 - protein, 50 aa, Homo sapiens; identical to SEQ ID NO: 5

SEQ ID NO: 8 - protein, 1359 aa, Homo sapiens
MSESEGGKGERILPSLQTLGASIVEWKPFDILILLTIFANCVALGVYIPFPEDDSNTANHNLEQVEYVFLVIFTVETVLKIVAYGLVLHPSAYIRNGWNLLDFIIVVVGLFSVLLEQGPGRPGDAPHTGGKPGGFDVKALRAFRVLRPLRLVSGVPSLHIVLNSIMKALVPLLHIALLVLFVIIIYAIIGLELFLGRMHKTCYFLGSDMEAEEDPSPCASSGSGRACTLNQTECRGRWPGPNGGITNFDNFFFAMLTVFQCVTMEGWTDVLYWMQDAMGYELPWVYFVSLVIFGSFFVLNLVLGVLSGEFSKEREKAKARGDFQKQREKQQMEEDLRGYLDWITQAEELDMEDPSADDNLGSMAEEGRAGHRPQLAELTNRRRGRLRWFSHSTRSTHSTSSHASLPASDTGSMTETQGDEDEEEGALASCTRCLNKIMKTRVCRRLRRANRVLQARCRRAVKSNACYWAVLLLVFLNTLTIASEHHGQPVWLTQIQEYANKVLLCLFTVEMLLKLYGLGPSAYVSSLFNRFDCFVVCGGILETTLVEVGAMQPLGISVLRCVRLLRIFKVTRHWASLSNLVASLLNSMKSIASLLLLLYLFIIIFSLLGMQLFGGKFNFDQTHTKRSTFDTFPQALLTVFQVLTGEDWNVVMYDGIMAYGGPFFPGMLVCIYFNILFICGNYILLNVFLAIAVDNLASGDAGTAKGKGGEKSNEKDLPQENEGLVPGVEKEEEEGARREGADMEEEEEEEEEEEEEEEEEGAGGVELLQEVVPKEKVVPIPEGSAFLCLSQTNPLRKGCHTLIHHHVFTNLILVFIILSSVSLAAEDPIRAHSFRNHILGYFDYAFTSIFTVEILLKMTVFGAFLHRGSFCRSWFNMLDLLVVSVSLISFGIHSSAISVVKILRVLRVLRPLRAINRAKGLKHVVQCVFVAIRTIGNIMIVTTLLQFMFACIGVQLFKGKFYTCTDEAKHTPQECKGSFLVYPDGDVSRPLVRERLWVNSDFNFDNVLSAMMALFTVSTFEGWPALLYKAIDAYAEDHGPIYNYRVEISVSFIVYIIIIAFFMMNIFVGFVIITFRAQGEQEYQNCELDKNQRQCVEYALKAQPLRRYIPKNPHQYRVWATVNSAAFEYLMFLLILLNTVALAMQHYEQTAPFNYAMDILNMVFTGLFTIEMVLKIIAFKPKMFGKVALQDGTQINRNNNFQTFPQAVLLLFRCATGEAWQEIMLASLPGNRCDPESDFGPGEEFTCGSNFAIAYFISFFMLCAFLGPHQTLGCGCPAETYPAPSGIWEAVPTPSGLQETCGNEHAPQLRWDGDIQRHTLCPGPDIPEDQNRREPGASQPGAADCHQKDLEADETEAAR

SEQ ID NO: 9 - protein, 1452 aa, Homo sapiens
MSESEGGKGERILPSLQTLGASIVEWKPFDILILLTIFANCVALGVYIPFPEDDSNTANHNLEQVEYVFLVIFTVETVLKIVAYGLVLHPSAYIRNGWNLLDFIIVVVGLFSVLLEQGPGRPGDAPHTGGKPGGFDVKALRAFRVLRPLRLVSGVPSLHIVLNSIMKALVPLLHIALLVLFVIIIYAIIGLELFLGRMHKTCYFLGSDMEAEEDPSPCASSGSGRACTLNQTECRGRWPGPNGGITNFDNFFFAMLTVFQCVTMEGWTDVLYWMQDAMGYELPWVYFVSLVIFGSFFVLNLVLGVLSGEFSKEREKAKARGDFQKQREKQQMEEDLRGYLDWITQAEELDMEDPSADDNLGSMAEEGRAGHRPQLAELTNRRRGRLRWFSHSTRSTHSTSSHASLPASDTGSMTETQGDEDEEEGALASCTRCLNKIMKTRVCRRLRRANRVLQARCRRAVKSNACYWAVLLLVFLNTLTIASEHHGQPVWLTQIQEYANKVLLCLFTVEMLLKLYGLGPSAYVSSLFNRFDCFVVCGGILETTLVEVGAMQPLGISVLRCVRLLRIFKVTRHWASLSNLVASLLNSMKSIASLLLLLYLFIIIFSLLGMQLFGGKFNFDQTHTKRSTFDTFPQALLTVFQVLTGEDWNVVMYDGIMAYGGPFFPGMLVCIYFNILFICGNYILLNVFLAIAVDNLASGDAGTAKGKGGEKSNEKDLPQENEGLVPGVEKEEEEGARREGADMEEEEEEEEEEEEEEEEEGAGGVELLQEVVPKEKVVPIPEGSAFLCLSQTNPLRKGCHTLIHHHVFTNLILVFIILSSVSLAAEDPIRAHSFRNHILGYFDYAFTSIFTVEILLKMTVFGAFLHRGSFCRSWFNMLDLLVVSVSLISFGIHSSAISVVKILRVLRVLRPLRAINRAKGLKHVVQCVFVAIRTIGNIMIVTTLLQFMFACIGVQLFKGKFYTCTDEAKHTPQECKGSFLVYPDGDVSRPLVRERLWVNSDFNFDNVLSAMMALFTVSTFEGWPALLYKAIDAYAEDHGPIYNYRVEISVSFIVYIIIIAFFMMNIFVGFVIITFRAQGEQEYQNCELDKNQRQCVEYALKAQPLRRYIPKNPHQYRVWATVNSAAFEYLMFLLILLNTVALAMQHYEQTAPFNYAMDILNMVFTGLFTIEMVLKIIAFKPKHYFTDAWNTFDALIVVGSIVDIAVTEVNSSEDSSRISITFFRLFRVMRLVKLLSKGEGIRTLLWTFIKSFQALPYVALLIAMIFFIYAVIGMQMFGKVALQDGTQINRNNNFQTFPQAVLLLFRCATGEAWQEIMLASLPGNRCDPESDFGPGEEFTCGSNFAIAYFISFFMLCAFLGPHQTLGCGCPAETYPAPSGIWEAVPTPSGLQETCGNEHAPQLRWDGDIQRHTLCPGPDIPEDQNRREPGASQPGAADCHQKDLEADETEAAR

SEQ ID NO: 10 - DNA, 21 bp, Artificial Sequence
  Primer
catactggaggaaagccagga

SEQ ID NO: 11 - DNA, 22 bp, Artificial Sequence
  Primer
tggagtgtgtggagcgagtaga

SEQ ID NO: 12 - DNA, 20 bp, Artificial Sequence
  Primer
gacgaatgcacaagacatgc

SEQ ID NO: 13 - DNA, 20 bp, Artificial Sequence
  Primer
caagcacaaggttgaggaca

SEQ ID NO: 14 - DNA, 21 bp, Artificial Sequence
  Primer
cgtccctcttcagcaagagaa

SEQ ID NO: 15 - DNA, 22 bp, Artificial Sequence
  Primer
catactggadggaaagccagga

SEQ ID NO: 16 - DNA, 25 bp, Artificial Sequence
  Primer
gcccataacttcgtataatgtatgc

SEQ ID NO: 17 - DNA, 20 bp, Artificial Sequence
  Primer
caagcacaaggttgaggaca